(19) United States
(12) Patent Application Publication
(10) Pub. No.: US 2024/0138838 A1
(43) Pub. Date: May 2, 2024

(54) SURFACE-MODIFIED ANASTOMOSIS DEVICE

(71) Applicant: Washington University, St. Louis, MO (US)

(72) Inventors: Xiaowei Li, St. Louis, MO (US); Justin Sacks, St. Louis, MO (US); William Moritz, St. Louis, MO (US); Emran Madatov, St. Louis, MO (US); Mohamed Zayed, St. Louis, MO (US)

(73) Assignee: Washington University, St. Louis, MO (US)

(21) Appl. No.: 18/317,780

(22) Filed: May 15, 2023

Related U.S. Application Data
(60) Provisional application No. 63/341,741, filed on May 13, 2022.

(51) Int. Cl. A61B 17/11; A61L 31/04; A61L 33/00; A61L 33/08

(52) U.S. Cl. CPC A61B 17/11; A61L 31/041; A61L 33/0011; A61L 33/08; A61B 2017/00893

(57) ABSTRACT

An anastomosis device for joining a first and second end of a vascular vessel is disclosed that includes a polymer cylinder ending in opposed first and second vascular-retaining ends configured for insertion into the first and second ends of the vascular vessel respectively. Each vascular-retaining end includes a plurality of bristles protruding outward. The device also includes at least one moiety covalently coupled to the surface of the device. The at least one moiety is selected from at least one anti-coagulant, at least one cell-specific binding peptide, and any combination thereof.

### CROSS-REFERENCE TO RELATED APPLICATIONS

This application claims priority from U.S. Provisional Application Ser. No. 63/341,741 filed on May 13, 2022, which is incorporated herein by reference in its entirety.

### STATEMENT REGARDING FEDERALLY SPONSORED RESEARCH OR DEVELOPMENT

Not applicable.

### MATERIAL INCORPORATED-BY-REFERENCE

The Sequence Listing, which is a part of the present disclosure, includes a computer-readable form comprising nucleotide and/or amino acid sequences of the present invention (file name “020046-US-NP_SEQUENCE_LISTING_CORRECTED.xml” created on 20 Nov. 2023; 45,267 bytes). The subject matter of the Sequence Listing is incorporated herein by reference in its entirety.

### FIELD OF THE INVENTION

The present disclosure generally relates to devices and methods for performing a suture-free anastomosis.

### BACKGROUND OF THE INVENTION

Microvascular or vascular anastomosis typically entails hand-sewing together blood vessels. It is a foundational surgical skill critical for plastic and reconstructive surgery, vascular surgery, transplant surgery, and more. Despite the rich history and a century of innovation, microvascular anastomosis still faces many challenges. Microvascular anastomosis (suturing of 1-3 mm blood vessels together) is a highly specialized surgical technique performed predominantly by surgeons possessing inherent dexterity and seven to eight years of training practice. Even when performed by highly skilled surgeons, inherent challenges in microvascular anastomosis can lead to 27% of cases with complications, and 25% involving reoperations. Anastomosis procedures are typically long, expensive, and require specialized operating resources, thus making them prohibitive in many hospitals. There is a current need for a simplified procedure or device that does not require specialized skills for efficient vascular anastomosis with comparable outcomes.

To date, the only commercially available device for microvascular anastomosis is the Global Excellence in Microsurgery (GEM) coupler (Synovis, Birmingham, AL), intended for use in anastomosis within the peripheral vascular system. Use of the GEM coupler is restricted to blood vessels with an outside diameter of 0.8˜4.3 mm and a wall thickness of 0.5 mm or less, rendering the GEM coupler suitable for venous, but not arterial, anastomosis. The GEM coupler implements extraluminal coupling that includes puncturing through the vessel wall, resulting in compromised vascular integrity. Other existing sleeve or cuff techniques utilizing absorbable biomaterials, such as poly(lactic acid), poly(lactic-co-glycolic acid), or poly(ε-caprolactone), are under development. These other technologies are also extraluminal and thus face the same use limitations as the GEM coupler. Typically, existing sleeve or cuff techniques for anastomosis are quite complicated because of their multiple pieces with the risk of fragmentation prior to vessel healing. A faster, easier, and safer alternative to current anastomosis devices is needed.

### SUMMARY OF THE INVENTION

In one aspect, an anastomosis device for joining a first and second end of a vascular vessel is disclosed that includes a polymer cylinder ending in opposed first and second vascular-retaining ends configured for insertion into the first and second ends of the vascular vessel respectively. Each vascular-retaining end includes a plurality of bristles protruding outward. The device also includes at least one moiety covalently coupled to the surface of the device. The at least one moiety is selected from at least one anti-coagulant, at least one cell-specific binding peptide, and any combination thereof. In some aspects, the polymer includes one of polyethylene terephthalate (PET), expanded polytetrafluoroethylene (ePTFE), and polyether ether-ketone (PEEK). In some aspects, the at least one anti-coagulant comprises heparin, a polysaccharide selected from cellulose, methylcellulose, hydroxymethyl cellulose, hydroxypropyl cellulose, hydroxypropylmethylcellulose, carboxymethylcellulose, sodium carboxymethylcellulose, cellulose sulfate, chitosan, chitosan salts, hyaluronic acid, hyaluronic acid salts, dextran sulfate, chondroitin sulfate, heparin, starch, any derivative thereof, and any combination thereof. In some aspects, the at least one cell-specific binding peptide comprises a peptide with a loop or linear structure that includes a sequence selected from REDV (SEQ ID NO:1), HGGVRLY (SEQ ID NO:2), RGD, IKVAV (SEQ ID NO:3), PDSGR (SEQ ID NO:4), and YIGSR (SEQ ID NO:5). In some aspects, the peptide with the loop structure comprises a sequence selected from SEQ ID NOS: 6-28. In some aspects, the peptide with the linear structure comprises a sequence selected from SEQ ID NOS: 29-51. In some aspects, the surface further includes surface modifications that include maleimide. In some aspects, the at least one moiety is covalently coupled to the maleimide.

Other objects and features will be in part apparent and in part pointed out hereinafter.

### DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1A is a graph of oscillatory time sweeps of HA hydrogels with F127-SS-DA at 10, 20, and 50 mg/mL.

FIG. 1B is a graph of oscillatory strain sweeps of HA hydrogels cross-linked by F127-SS-DA.

FIG. 1C is a graph of oscillatory time sweeps of HA hydrogels with F127-DA at 10, 20, and 50 mg/mL.

FIG. 1D is a graph of oscillatory strain sweeps of HA hydrogels cross-linked by F127-DA.

FIG. 1E is a table of HA hydrogel gelation time.

FIG. 1F is a graph of reduced H₂O₂ with hydrogels containing F127-SS-DA or F127-DA at 10, 20, and 50 mg/mL polymer. The HA hydrogels with F127-SS-DA consumed significantly more H₂O₂ than those with F127-DA. N=3. *0.01<P<0.05, **0.001<P<0.01, ***0.0001<P<0.001, and ****P<0.0001.

FIG. 1G is a graph of DPPH scavenging efficiency with hydrogels containing F127-SS-DA or F127-DA at 10, 20, and 50 mg/mL polymer. HA hydrogels with F127-SS-DA showed higher DPPH scavenging effects than those with F127-DA at the same concentration. N=3. *0.01<P<0.05, **0.001<P<0.01, ***0.0001<P<0.001, and ****P<0.0001.

FIG. 2A is a set of images of vascular-like networks formed on the surface of HA hydrogels with various concentrations of RGD (0, 0.01, 0.1, and 1 mM). Matrigel was used as a positive control. LIVE: green and DEAD: red. Scale bar=200 μm.

FIG. 2B is a graph of quantitative analysis of branch density from images in FIG. 2A. N=3˜6. *0.01<P<0.05, ***0.0001<P<0.001, and ****P<0.0001.

FIG. 2C is a graph of quantitative analysis of total branch length from images in FIG. 2A. N=3˜6. *0.01<P<0.05, ***0.0001<P<0.001, and ****P<0.0001.

FIG. 2D is a graph of quantitative analysis of mean mesh size from images in FIG. 2A. N=3˜6. *0.01<P<0.05, ***0.0001<P<0.001, and ****P<0.0001.

FIG. 2E is a set of images of vascular-like networks formed inside the HA hydrogels with various concentrations of RGD (0, 0.10, 0.25, and 0.50 mM). Phalloidin 568 for F-actin: red and DAPI for nuclei: blue. Scale bar=100 μm.

FIG. 2F is a graph of quantitative analysis of branch density from images in FIG. 2E. N=3˜6. *0.01<P<0.05, ***0.0001<P<0.001, and ****P<0.0001.

FIG. 2G is a graph of quantitative analysis of total branch length from images in FIG. 2E. N=3˜6. *0.01<P<0.05, ***0.0001<P<0.001, and ****P<0.0001.

FIG. 2H is a graph of quantitative analysis of mean mesh size from images in FIG. 2E. N=3˜6. *0.01<P<0.05, ***0.0001<P<0.001, and ****P<0.0001.

FIG. 3A is a set of images of vascular-like networks formed inside HA hydrogels with F127-SSDA or F127-DA at 0, 2, 4, 8, 10, 20, and 50 mg/mL, respectively. HA: 4 mg/mL. RGD: 0.1 mM. Phalloidin 568 for F-actin: red and DAPI for nuclei: blue. Scale bar=100 μm.

FIG. 3B is a graph of quantitative analysis of branch density from images in FIG. 3A. N=3˜6. *0.01<P<0.05, ***0.0001<P<0.001, and ****P<0.0001.

FIG. 3C is a graph of quantitative analysis of total branch length from images in FIG. 3A. N=3˜6. *0.01<P<0.05, ***0.0001<P<0.001, and ****P<0.0001.

FIG. 3D is a graph of quantitative analysis of mean mesh size from images in FIG. 3A. N=3˜6. *0.01<P<0.05, ***0.0001<P<0.001, and ****P<0.0001.

FIG. 3E is a graph of G′ of HA hydrogels with F127-SS-DA or F127-DA. N=3.

FIG. 3F is a graph of the DPPH scavenging efficiency of HA hydrogels with F127-SS-DA or F127-DA, respectively. N=3-4. *0.01<P<0.05, ***0.0001<P<0.001, and ****P<0.0001.

FIG. 4A is a set of images of HUVECs and hASCs that were cocultured inside HA hydrogels under H₂O₂ conditions (0 and 1 mM). Alexa Fluor phalloidin 48 for F-actin: green and DAPI for nuclei: blue. Scale bar=100 μm.

FIG. 4B is a graph of quantitative analysis of branch density from images in FIG. 4A. N=3. *0.01<P<0.05, ***0.0001<P<0.001, and ****P<0.0001.

FIG. 4C is a graph of quantitative analysis of total branch length from images in FIG. 4A. N=3. *0.01<P<0.05, ***0.0001<P<0.001, and ****P<0.0001.

FIG. 4D is a graph of quantitative analysis of mean mesh size from images in FIG. 4A. N=3. *0.01<P<0.05, ***0.0001<P<0.001, and ****P<0.0001.

FIG. 4E is a graph of intracellular ROS levels with HA hydrogels under H₂O₂ conditions (0, 1.0, 2.5, and 5.0 mM). ROS levels were quantified by fluorescence intensity. N=3. *0.01<P<0.05, **0.001<P<0.01.

FIG. 5A is a set of images of cell-containing hydrogels with MT staining showing functional recovery of rats with ischemic limbs after the treatment with HA hydrogels. HA hydrogels were identified between myofascicles, as shown by a star mark. Scale bar=200 μm.

FIG. 5B is a set of images of cell-containing hydrogels with H&E staining showing functional recovery of rats with ischemic limbs after the treatment with HA hydrogels. HA hydrogels were identified between myofascicles, as shown by a star mark. Scale bar=200 μm.

FIG. 5C is a set of images of cell-containing hydrogels with immunostaining of CD31-positive endothelial cells to identify capillaries inside the rat gastrocnemius showing functional recovery of rats with ischemic limbs after the treatment with HA hydrogels. HA hydrogels were identified between myofascicles, as shown by a star mark. Scale bar=200 μm.

FIG. 5D is a graph of the maximum running distance evaluated by treadmill running tests. N=4-6. *0.01<P<0.05, **0.001<P<0.01, ***0.0001<P<0.001.

FIG. 5E is a graph of the quantification of MDA in the rat gastrocnemius after treatments with the HA hydrogels. PBS. HA: hydrogel. H/VEGF: hydrogel with the VEGF. N=4-6. *0.01<P<0.05, **0.001<P<0.01, ***0.0001<P<0.001.

FIG. 6A is a heat map analysis of gene expression of hydrogel-mediated expression of oxidative stress-related genes inside the rat gastrocnemius.

FIG. 6B is a graph of a comparison of fold regulation of gene expression.

FIG. 6C is a table of specific genes with a significantly high expression. N=4-6. *0.01<P<0.05, **0.001<P<0.01, ***0.0001<P<0.001.

FIG. 6D is a table of specific genes with a significantly low expression. N=4-6. *0.01<P<0.05, **0.001<P<0.01, ***0.0001<P<0.001.

FIG. 7 is a schematic of the process involved in HA hydrogel-mediated oxidative stress regulation for functional recovery of rats with ischemic hindlimbs.

FIG. 8 is a set of images of HA hydrogels conjugated with RGD-supported HUVECs to form vascular-like networks. LIVE: green and DEAD: red. Scale bar=1 mm.

FIG. 9 is a graph of the shear storage moduli (G′) of HA hydrogels with different RGD at 0, 0.10, 0.25, and 0.50 mM.

FIG. 10 is a graph of the cumulative release profiles of VEGF from HA hydrogels in vitro. N=3.

FIG. 11 is a set of images of HA hydrogels identified inside the rat gastrocnemius, including images with Masson's Trichrome (MT) staining, H&E staining, and immunostaining of CD31-positive endothelial cells to identify capillary inside the rat gastrocnemius. Hydrogels were identified between myofascicles as shown by a star mark. H: Hydrogel. H/VEGF: Hydrogel with VEGF. Scale bar=200 μm.

FIG. 12A is a graph of the relative local blood flow velocity at the proximal site inside the gastrocnemius investigated by a Transonic Tissue Perfusion Monitor with a monofiber probe. N=4-6. *0.01<P<0.05, **0.001<P<0.01.

FIG. 12B is a graph of the relative local blood flow velocity at the distal site inside the gastrocnemius investigated by a Transonic Tissue Perfusion Monitor with a monofiber probe. N=4-6. *0.01<P<0.05, **0.001<P<0.01.

FIG. 13A is a set of graphs of the swelling and degradation ratio over time in HA hydrogels under H2O2 conditions. H2O2: 0, 0.5, 10, and 100 mM. N=3.

FIG. 13B is a graph of the DPPH scavenging efficiency for HA hydrogels crosslinked by PEGDA or PEGSSDA. N=3. ***0.0001<P<0.001.

FIG. 14 is a graph of the surface modification of PEEKs with carboxylic acid (COOH) groups. PEEKs were treated by air plasma for 5, 10, 15, and 30 minutes. The COOH groups on the surface of plasma-treated PEEKs were detected by toluidine blue assay.

FIG. 15A is a schematic of the process of surface modification of PEEKs with RGD to support endothelial cell attachment.

FIG. 15B is a set of images cell containing HA hydrogels. MAL-Modified and RGD-Modified PEEK showed greater endothelial cell attachment than Non-Modified PEEK (Scale bar=100 μm).

FIG. 15C is a graph of the reduction of AlamarBlue in HA hydrogels. A significantly larger number of endothelial cells with MAL- and RGD-Modified PEEK compared to Non-Modified PEEK was confirmed by the AlamarBlue assay.

FIG. 16A is an image of the internal carotid artery and internal jugular vein from a Vaso-Lock for arterio-venous anastomosis procedure.

FIG. 16B is an image of the Vaso-Lock placed in an internal carotid artery for an arterio-venous anastomosis procedure.

FIG. 16C is an image of the arterio-venous anastomosis with Vaso-Lock for an arterio-venous anastomosis procedure.

FIG. 16D is an image of the anastomosis that remained in place after clamp removal from a Vaso-Lock during an arterio-venous anastomosis procedure.

FIG. 16E is an arteriogram that showed patency of anastomosis without contrast extravasation using a Vaso-Lock for an arterio-venous anastomosis procedure.

FIG. 16F is a Doppler ultrasonography for anastomosis by Vaso-Lock.

FIG. 17A is a perspective view showing the design of a Vaso-Lock sutureless anastomosis device.

FIG. 17B is a close-up image of the Bristles on the Vaso-Lock of FIG. 17A.

FIG. 17C is an image showing 3D-printed Vaso-Locks with diameters ranging from 2 to 5 mm.

FIG. 17D is an image comparing a hand-sewn anastomosis and a Vaso-Lock (3 mm in diameter) anastomosis for porcine carotid; the Vaso-Lock anastomosis (right) had higher tensile strength than the hand-sewn anastomosis (left).

FIG. 17E is a bar graph comparing the tensile strength of the hand-sewn and Vaso-Lock anastomoses depicted in FIG. 17D.

FIG. 17F is an image of a preparation illustrating the Vaso-Lock anastomosis of FIG. 17D experienced no leakage as visualized by red saline under physiologic flow rates.

FIG. 18A is a set of images of vascular-like networks formed on the surface of hyaluronic acid (HA) hydrogels with various concentrations of RGD (0, 0.01, 0.1, and 1 mM). Matrigel was used as a positive control. LIVE: green and DEAD: red. Scale bar=200 μm.

FIG. 18B is a graph quantifying vascular branch density in the hydrogels seen in FIG. 18A. N=3˜6. #P<0.05.

FIG. 18C is a graph quantifying the total branch length in the hydrogels seen in FIG. 18A. N=3˜6. #P<0.05.

FIG. 18D is a graph quantifying the mean mesh size in the hydrogels seen in FIG. 18A. N=3˜6. #P<0.05.

FIG. 18E is a set of images of vascular-like networks formed inside the HA hydrogels with various concentrations of RGD (0, 0.10, 0.25, and 0.50 mM). Phalloidin 568 for F-actin: red and DAPI for nuclei: blue. Scale bar=100 μm.

FIG. 18F is a graph quantifying branch density in the hydrogels seen in FIG. 18E. N=3˜6. *P<0.05, ***P<0.001, and ****P<0.0001.

FIG. 18G is a graph quantifying the total branch length in the hydrogels seen in FIG. 18E. N=3˜6. *P<0.05, ***P<0.001, and ****P<0.0001.

FIG. 18H is a graph quantifying the mean mesh size in the hydrogels seen in FIG. 18E. N=3˜6. *P<0.05, ***P<0.001, and ****P<0.0001.

FIG. 19A is a schematic of an ammonia plasma treatment of PEEKs.

FIG. 19B is a set of XPS survey spectra for PEEKs for plasma treatment with 0, 1, 10, and 20 min.

FIG. 19C is a set of high-resolution XPS spectra of C1 s.

FIG. 19D is a set of high-resolution XPS spectra of O1s.

FIG. 19E is a set of high-resolution XPS spectra of N1 s.

FIG. 19F is a table of atomic composition (%) of PEEKs after ammonia plasma treatment with 0, 1, 10, and 20 min.

FIG. 20A is a schematic of PEEK modification with peptides HGGVRLY and REDV.

FIG. 20B is a set of XPS survey spectra for the modified PEEKs described in FIG. 20A.

FIG. 20C is a set of high-resolution XPS spectra of C1 s.

FIG. 20D is a set of high-resolution XPS spectra of O1s.

FIG. 20E is a set of high-resolution XPS spectra of N1 s.

FIG. 20F is a set of high-resolution XPS spectra of S1s.

FIG. 20G is a table quantifying the atomic composition (%) of the modified PEEK surfaces described in FIG. 20A.

FIG. 20H is a graph of water contact angles of all PEEKs. A significantly larger water contact angle of non-modified PEEK compared to other modified groups was observed. *P<0.05.

FIG. 21A is a set of SEM images of HUVECs on the surface of PEEKs. A monolayer of endothelial cells appeared on the surface of cyclic HGGVRLY-modified PEEK on Day 21.

FIG. 21B is a graph quantifying an AlamaBlue assay of cell proliferation on the PEEKs. A significantly larger number of HUVECs showed on the cyclic and linear HGGVRLY-modified PEEKs compared to the non-modified PEEK on Day 21. Cyclic REDV-modified PEEK also supported a larger number of HUVECs when compared to the linear REDV-modified PEEK or the non-modified PEEK. *P<0.05.

FIG. 22A is a set of images of a co-culture of HUVECs and hSMCs on the surface of PEEKs on Day 1.

FIG. 22B is a graph quantifying the number of attached HUVECs and smooth muscle cells on Day 1. A larger number of HUVECs than hSMCs appeared on the surface of cyclic HGGVRLY-modified PEEK. *P<0.05.

FIG. 22C is a set of images showing endothelium formation on the surface of PEEKs on Day 14.

FIG. 22D is a graph quantifying an AlamaBlue assay of cell proliferation on the PEEKs. At Days 1 and 5, a large number of cells appeared on the cyclic HGGVRLY-modified PEEK compared to the plasma- and non-modified PEEKs. At Days 8 and 12, the linear and cyclic HGGVRLY-modified PEEK showed a larger number of cells compared to the plasma- and non-modified PEEKs. At Day 15, cyclic REDV-, linear HGGVRLY-, and cyclic HGGVRLY-modified PEEKs showed a larger number of cells compared to the plasma- and non-modified PEEKs. *P<0.05.

FIG. 23A is a set of typical SEM images of adhered platelets on the PEEKs after incubation with pig platelet-rich plasma.

FIG. 23B is a graph quantifying platelet density on the PEEKs seen in FIG. 23A. A larger number of platelets was observed on the plasma-modified PEEK when compared to the linear or cyclic HGGVRLY-modified PEEKs. *P<0.05.

FIG. 24A is a schematic of a Vaso-Lock designed with anchors.

FIG. 24B is an image of an injection-molded Vaso-Lock.

FIG. 24C is a representative scanning electron microscope image of an anchor of the Vaso-Lock before ethylene oxide sterilization.

FIG. 24D is a representative scanning electron microscope image of the outer surface of the Vaso-Lock before ethylene oxide sterilization.

FIG. 24E is a representative scanning electron microscope image of an anchor of the Vaso-Lock before ethylene oxide sterilization.

FIG. 24F is a representative scanning electron microscope image of the inner surface of the Vaso-Lock before ethylene oxide sterilization.

FIG. 24G is a representative scanning electron microscope image of an anchor of the Vaso-Lock after ethylene oxide sterilization.

FIG. 24H is a representative scanning electron microscope image of the outer surface of the Vaso-Lock after ethylene oxide sterilization.

FIG. 24I is a representative scanning electron microscope image of an anchor of the Vaso-Lock after ethylene oxide sterilization.

FIG. 24J is a representative scanning electron microscope image of the inner surface of the Vaso-Lock after ethylene oxide sterilization.

FIG. 25A is an image of a Vaso-Lock and hand-sewn anastomosis of the swine common carotid artery and internal jugular vein. This image shows the common carotid artery and internal jugular vein identified for anastomosis by Vaso-Lock. Scale bar: 10 mm.

FIG. 25B is an image of the Vaso-Lock placed in the common carotid artery. Scale bar: 10 mm.

FIG. 25C is an image of an arterio-venous anastomosis with Vaso-Lock. Scale bar: 10 mm.

FIG. 25D is an image of a hand-sewn anastomosis. The common carotid artery and internal jugular vein were identified for hand-sewn anastomosis. Scale bar: 10 mm.

FIG. 25E is an image of hand-sewn anastomosis wherein sutures are applied for anastomosis. Scale bar: 10 mm.

FIG. 25F is an image of a hand-sewn anastomosis wherein arterio-venous anastomosis by sutures is performed. Scale bar: 10 mm.

FIG. 26A is a set of Doppler ultrasonography images of Vaso-Lock for anastomosis for 2-week pigs.

FIG. 26B is a set of Doppler ultrasonography images of Vaso-Lock for anastomosis for 6-week pigs.

FIG. 26C is a pair of images wherein blood velocity was checked by pulse wave spectrum within the Vaso-Lock after implantation at Week 0.

FIG. 26D is a pair of images wherein blood velocity was checked by pulse wave spectrum within the Vaso-Lock after implantation at Week 4.

FIG. 26E is a set of Doppler ultrasonography images of hand-sewn anastomosis for 2-week pigs.

FIG. 26F is a set of Doppler ultrasonography images of hand-sewn anastomosis for 6-week pigs.

FIG. 26G is a pair of images wherein blood velocity was checked at the hand-sewn anastomosis site at Week 0.

FIG. 26H is a pair of images wherein blood velocity was checked at the hand-sewn anastomosis site at Week 4.

FIG. 27A is an image using Vaso-Lock for anastomosis. Flowing blood can be identified in the common carotid artery.

FIG. 27B is another image using Vaso-Lock for anastomosis. Flowing blood can be identified in the common carotid artery.

FIG. 27C is another image using Vaso-Lock for anastomosis. The vessel lumen can be viewed at the common carotid artery side.

FIG. 27D is another image using Vaso-Lock for anastomosis. The guidewire went through the explanted blood vessels anastomosed by the Vaso-Lock.

FIG. 27E is an image of a hand-sewn anastomosis. The vessel lumen is viewed at the common carotid artery.

FIG. 27F is another image of a hand-sewn anastomosis. The vessel lumen is viewed at the internal jugular vein side.

FIG. 27G is another image of a hand-sewn anastomosis, showing the guidewire that went through the explanted blood vessels anastomosed by sutures.

FIG. 28A is a set of H&E stained images of explanted blood vessels at Weeks 2 and 6.

FIG. 28B is a set of Masson's Trichrome stained images of explanted blood vessels at Weeks 2 and 6. Collagen is stained in blue.

FIG. 28C is a set of Verhoeff-Van Gieson stained images of explanted blood vessels at Weeks 2 and 6.

FIG. 28D is a set of Alizarin Red stained images of explanted blood vessels at Weeks 2 and 6. Alizarin Red staining. Calcium accumulation (dark red) was identified for the Vaso-Lock sample at Week 2, and hand-sewn samples at Weeks 2 and 6, respectively. Calcium was quantified and indicated in the figure.

FIG. 29A is a schematic for Vaso-Lock for anastomosis.

FIG. 29B is a schematic for hand-sewn anastomosis.

FIG. 29C is a pair of images of tissue blocks that indicated that Vaso-Lock joined blood vessels by anchors (arrows) at Weeks 2 and 6. Scale bar: 2 mm.

FIG. 29D is an image of a normal common carotid artery with a uniform tunica media. Scale bar: 2 mm.

FIG. 29E is an image of anastomosed blood vessels with a maintained open lumen by Vaso-Lock at Week 2. Scale bar: 2 mm.

FIG. 29F is an image of anastomosed blood vessels with a maintained open lumen by hand-sewn technique at Week 2. Scale bar: 2 mm.

FIG. 29G is a set of images of a series of tissue sections of blood vessels anastomosed by Vaso-Lock at Week 6. Scale bar: 2 mm.

FIG. 29H is a set of images of a series of tissue sections of blood vessels anastomosed by hand-sewn technique at Week 6. Scale bar: 2 mm.

FIG. 29I is a plot of the quantification of tunica media of tissue sections at Week 6. Intimal growth was identified at the arterial side.

FIG. 29J is a set of images showing that the anchors of the Vaso-Lock penetrated the tunica intima into media. Smooth muscle cells stained with αSMA in green. Endothelial cells stained with CD31 in red. Cell nuclei stained with DAPI in blue. Scale bar: 200 μm.

FIG. 29K is another set of images showing that the anchors of the Vaso-Lock penetrated the tunica intima within the media. Smooth muscle cells stained with αSMA in green. Endothelial cells stained with CD31 in red. Cell nuclei stained with DAPI in blue. Scale bar: 200 μm.

FIG. 29L is a set of images showing sutures identified in the middle layer of the common carotid artery. Smooth muscle cells stained with αSMA in green. Endothelial cells stained with CD31 in red. Cell nuclei stained with DAPI in blue. Scale bar: 200 μm.

FIG. 29M is another set of images showing sutures identified in the middle layer of the common carotid artery. Smooth muscle cells stained with αSMA in green. Endothelial cells stained with CD31 in red. Cell nuclei stained with DAPI in blue. Scale bar: 200 μm.

FIG. 30A is a set of images of Vaso-Lock for anastomosis at Week 2. Smooth muscle cells stained with αSMA in red. TGFβ1 stained in green. Cell nuclei stained with DAPI in blue. Scale bar: 200 μm.

FIG. 30B is another set of images of Vaso-Lock for anastomosis at Week 2. Smooth muscle cells stained with αSMA in red. TGFβ1 stained in green. Cell nuclei stained with DAPI in blue. Scale bar: 200 μm.

FIG. 30C is a set of images of hand-sewn anastomosis at Week 2. Smooth muscle cells stained with αSMA in red. TGFβ1 stained in green. Cell nuclei stained with DAPI in blue. Scale bar: 200 μm.

FIG. 30D is another set of images of hand-sewn anastomosis at Week 2. Smooth muscle cells stained with αSMA in red. TGFβ1 stained in green. Cell nuclei stained with DAPI in blue. Scale bar: 200 μm.

FIG. 30E is a graph of the quantification of TGFβ1 expression at Weeks 2. *P<0.05.

FIG. 30F is a set of images of Vaso-Lock for anastomosis at Week 6. Smooth muscle cells stained with αSMA in red. TGFβ1 stained in green. Cell nuclei stained with DAPI in blue. Scale bar: 200 μm.

FIG. 30G is another set of images of Vaso-Lock for anastomosis at Week 6. Smooth muscle cells stained with αSMA in red. TGFβ1 stained in green. Cell nuclei stained with DAPI in blue. Scale bar: 200 μm.

FIG. 30H is a set of images of hand-sewn anastomosis at Week 6. Smooth muscle cells stained with αSMA in red. TGFβ1 stained in green. Cell nuclei stained with DAPI in blue. Scale bar: 200 μm.

FIG. 30I is another set of images of hand-sewn anastomosis at Week 6. Smooth muscle cells stained with αSMA in red. TGFβ1 stained in green. Cell nuclei stained with DAPI in blue. Scale bar: 200 μm.

FIG. 30J is a graph of the quantification of TGFβ1 expression at Week 6. * P<0.05.

FIG. 31A is a set of images showing proliferation in Vaso-Lock for anastomosis at Week 2. Proliferated cells stained with Ki67 in red. Smooth muscle cells stained with αSMA in green. Cell nuclei stained with DAPI in blue. Scale bar: 200 μm.

FIG. 31B is another set of images showing proliferation in Vaso-Lock for anastomosis at Week 2. Proliferated cells stained with Ki67 in red. Smooth muscle cells stained with αSMA in green. Cell nuclei stained with DAPI in blue. Scale bar: 200 μm.

FIG. 31C is a set of images showing proliferation in hand-sewn anastomosis at Week 2. Proliferated cells stained with Ki67 in red. Smooth muscle cells stained with αSMA in green. Cell nuclei stained with DAPI in blue. Scale bar: 200 μm.

FIG. 31D is another set of images showing proliferation in hand-sewn anastomosis at Week 2. Proliferated cells stained with Ki67 in red. Smooth muscle cells stained with αSMA in green. Cell nuclei stained with DAPI in blue. Scale bar: 200 μm.

FIG. 31E is a graph of the quantification of the proliferated cells at Weeks 2. *P<0.05.

FIG. 31F is a set of images showing proliferation in Vaso-Lock for anastomosis at Week 6. Proliferated cells stained with Ki67 in red. Smooth muscle cells stained with αSMA in green. Cell nuclei stained with DAPI in blue. Scale bar: 200 μm.

FIG. 31G is another set of images showing proliferation in Vaso-Lock for anastomosis at Week 6. Proliferated cells stained with Ki67 in red. Smooth muscle cells stained with αSMA in green. Cell nuclei stained with DAPI in blue. Scale bar: 200 μm.

FIG. 31H is a set of images showing proliferation in hand-sewn anastomosis at Week 6. Proliferated cells stained with Ki67 in red. Smooth muscle cells stained with αSMA in green. Cell nuclei stained with DAPI in blue. Scale bar: 200 μm.

FIG. 31I is another set of images showing proliferation in hand-sewn anastomosis at Week 6. Proliferated cells stained with Ki67 in red. Smooth muscle cells stained with αSMA in green. Cell nuclei stained with DAPI in blue. Scale bar: 200 μm.

FIG. 31J is a graph of the quantification of the proliferated cells at Week 6. *P<0.05.

FIG. 32A is a set of images of macrophages at the Vaso-Lock site at Week 2. Macrophages were identified by CD68 (Pan macrophages), CD86 (Inflammatory macrophages), and CD206 (pro-regenerative macrophages). Scale bar: 200 μm.

FIG. 32B is a set of images of macrophages at the hand-sewn anastomosis site at Week 2. Macrophages were identified by CD68 (Pan macrophages), CD86 (Inflammatory macrophages), and CD206 (pro-regenerative macrophages). Scale bar: 200 μm.

FIG. 32C is a set of images of macrophages at the Vaso-Lock site at Week 6. Macrophages were identified by CD68 (Pan macrophages), CD86 (Inflammatory macrophages), and CD206 (pro-regenerative macrophages). Scale bar: 200 μm.

FIG. 32D is a set of images of macrophages at the hand-sewn anastomosis site at Week 6. Macrophages were identified by CD68 (Pan macrophages), CD86 (Inflammatory macrophages), and CD206 (pro-regenerative macrophages). Scale bar: 200 μm.

FIG. 32E is a graph quantifying the % of CD68+ cells in Vaso-Lock and hand-sewn anastomosis sites at Week 2 from the images described in FIG. 32A-B. *P<0.05.

FIG. 32F is a graph quantifying the % of CD68+ cells in Vaso-Lock and hand-sewn anastomosis sites at Week 6 from the images described in FIG. 32C-D. *P<0.05.

FIG. 32G is a graph quantifying the % of CD86+ cells in Vaso-Lock and hand-sewn anastomosis sites at Week 2 from the images described in FIG. 32A-B. *P<0.05.

FIG. 32H is a graph quantifying the % of CD86+ cells in Vaso-Lock and hand-sewn anastomosis sites at Week 6 from the images described in FIG. 32C-D. *P<0.05.

FIG. 32I is a graph quantifying the % of CD206+ cells in Vaso-Lock and hand-sewn anastomosis sites at Week 2 from the images described in FIG. 32A-B. *P<0.05.

FIG. 32J is a graph quantifying the % of CD206+ cells in Vaso-Lock and hand-sewn anastomosis sites at Week 6 from the images described in FIG. 32C-D. *P<0.05.

FIG. 33A is a set of H&E images from various organs of pigs with Vaso-Lock for anastomosis at Week 6.

FIG. 33B is a set of H&E images from various organs of pigs with hand-sewn anastomosis at Week 6.

FIG. 34 is a table of peptides with cyclic structures and their reactive groups that are used in Example 3.

### DETAILED DESCRIPTION OF THE INVENTION

The present disclosure is based, at least in part, on the discovery that surface modification of intravascular devices with peptides derived from fibronectin enhanced epithelial cell attachment and development, thereby providing for antithrombogenetic and sutureless anastomosis.

Among the various aspects of the present disclosure is the provision of devices and methods for performing a suture-free anastomosis. In various aspects, the device includes a polymer cylinder ending in opposed first and second vascular-retaining ends configured for insertion into the first and second ends of the vascular vessel respectively. Each vascular-retaining end is provided with a plurality of bristles protruding outward; the bristles are configured to retain the ends of the vascular vessel intraluminally without the use of sutures. In addition, the device further includes at least one functional moiety covalently coupled to the surfaces of the device including, but not limited to, an anti-coagulant, a cell-specific binding peptide, and any combination thereof.

In various other aspects, a method of performing an anastomosis using the disclosed device is disclosed. The disclosed method includes inserting the first and second vascular-retaining ends of the disclosed device into the lumen of the vascular vessel at the first and second ends of the vascular vessel, thereby rejoining the vascular vessel at the cut ends. In various aspects, the outward-projecting bristles of the first and second vascular-retaining ends retain the corresponding vascular vessel ends without the need for sutures, adhesives, or any other fastening means. In various other aspects, the moieties are attached to the surfaces of the device. In another aspect, the device can be a polymer cylinder.

The disclosed anastomosis overcome at least several limitations associated with existing anastomosis devices and methods. As discussed herein, the disclosed device provides for the joining of the vascular vessel ends by insertion of the vascular-retaining ends into the lumen of the cut vessel ends, thereby eliminating the need for fine suturing requiring highly skilled and trained practitioners. Further, as described in the Examples below, anastomoses performed using the disclosed device exhibited higher joining strength as compared to corresponding suture-based anastomoses. In addition, the attached moieties enhance epithelialization of the anastomosis, as well as reduce the risk of complications such as thrombosis.

In various aspects, the use of the disclosed device, including, but not limited to, the anti-thrombogenic sutureless Vaso-Lock devices, changes the paradigm of surgical training and practice, and improves technical capabilities for vascular anastomosis. The disclosed devices and methods simplify a traditionally complex surgical technique, improve patient outcomes and safety, and make anastomosis more globally available.

In various additional aspects, the disclosed devices and methods may be modified to provide for the implementation of other endoluminal anastomoses including ureter, fallopian tubes, biliary trees, and any other suitable procedure without limitation In various additional aspects, the disclosed devices and methods are suitable for use by a variety of surgeons, including, but not limited to, plastic and reconstructive surgeons, vascular surgeons, cardiothoracic surgeons, neurosurgeons, otolaryngologists, and oral and maxillofacial surgeons, and includes the performance of both in-patient and out-patient procedures.

### Anastomosis Coupling Device

In various aspects, anastomosis coupling devices are disclosed. Non-limiting examples of suitable anastomosis coupling devices include sutureless anastomosis devices such as the 3D-printed Vaso-Lock device. In various aspects, the Vaso-Lock is used as a coupler to hold free vascular ends together with traction by bristles, delivering consistent, expeditious anastomosis. Because the disclosed devices are inserted intraluminally, the bristles do not penetrate the vessel wall. Instead, the bristles exert forces against the vessel walls, utilizing the elasticity of the vessels to hold the vessel in place with a tight seal, obviating the need for additional sutures or adhesives. 3D-printing technology has been utilized to prototype the disclosed couplers with various diameters (2-5 mm), which provides for quick adjustments in design while further providing for ready customization, cost-effective prototyping, and fast production. When compared to the existing GEM coupler, Vaso-Lock is unrestricted with respect to size and can be used for super microsurgery and vascular surgery.

As demonstrated in the Examples below, a Vaso-Lock device can be deployed in porcine carotid arteries within 1 minute, while hand-sewn anastomosis in arteries can take around 1 hour by a proficiently trained surgeon. The Vaso-Lock displayed higher tensile strength than that of hand-sewn anastomosis (6.3 vs. 4.9 N). Using a bioreactor cannula with a pulsatile pump, Vaso-Lock anastomosis of porcine carotid arteries withstood flow rates up to 45 mL/min without leakage (physiologic pulsatile flow is approximately 15 m L/m in).

Non-limiting examples of suitable anastomosis coupling devices include Vaso-Lock devices as described in PCT Application Publication No. WO 2022/011053, the content of which is incorporated by reference in its entirety.

Materials

In various aspects, any suitable biomaterial may be used in the construction of the disclosed anastomosis devices without limitation. Non-limiting examples of suitable biomaterials include any material used in the construction of synthetic vascular devices. Other non-limiting examples of suitable biomaterials include polymers such as polyethylene terephthalate (PET), expanded polytetrafluoroethylene (ePTFE), and polyether ether-ketone (PEEK), which are widely used for synthetic vascular devices. In one aspect, the biomaterial used in the construction of the disclosed anastomosis devices is polyether ether-ketone (PEEK).

When compared to autologous vascular grafts, the biomaterials disclosed herein exhibit lower patency but are still suitable for large-diameter applications (>6 mm). However, as applied to small-diameter vessels (<6 mm), these synthetic vascular devices exhibit poor long-term patency. In particular, the low blood flow rate inside the small-diameter vessels and turbulent flow at the sites of anastomosis lead to a high risk of luminal thrombosis. When upon exposure to synthetic vascular devices, blood defense mechanisms are activated. They involve activation of the coagulation cascade, complement system, and cellular inflammatory mechanisms. Specifically, nonspecific protein adsorption is the first step, which initiates platelet adhesion, activation, and clot formation. To prevent thrombosis, synthetic vascular devices should exhibit high resistance to nonspecific protein adsorption. Furthermore, the risk of thrombosis is still present if the surface is not fully covered by an endothelial cell layer. A healthy endothelium monitors the behavior of surrounding cells through various signaling mechanisms for vessel integrity. The loss of signaling in damaged endothelium is a prominent contributor to the unregulated hyperproliferation of smooth muscle cells, which leads to intimal hyperplasia and vessel occlusion. Here we have selected PEEK for 3D-printing Vaso-Locks. PEEK is an FDA-approved biologically inert material, causing neither toxic nor mutagenic effects nor clinically significant inflammation. In various aspects, the surfaces of the PEEK-based Vaso-Locks are modified as described herein to prevent thrombogenic protein absorption and support full endothelium formation.

Surface Modifications

In various aspects, the surfaces of the disclosed devices are modified through the attachment of at least one moiety to enhance hemocompatibility and support endothelialization. Various approaches have been explored to modify the surface of synthetic vascular devices, such as covalently linking heparin, antiplatelet agents, thrombolytic agents, or hydrophilic polymers. In various aspects, the incorporation of heparin into the disclosed device is an effective way to improve antithrombogenicity because of the excellent anticoagulation properties of heparin. Platelet adhesion has been shown to be significantly reduced on different heparin-modified materials, such as PTFE and decellularized matrices. Heparin is preferentially immobilized on the device surface because heparin may gradually release and cause low sustainability in long-term implantation applications.

In various other aspects, cell-specific binding peptides are applied to pretreat synthetic vascular grafts to support cell attachment and retention. Various peptide sequences have been discovered, such as REDV (SEQ ID NO:1), HGGVRLY (SEQ ID NO:2), and RGD from fibronectin, as well as IKVAV (SEQ ID NO:3), PDSGR (SEQ ID NO:4), and YIGSR (SEQ ID NO:5) as laminin-derived recognition sequences.

In various aspects, cell-specific binding peptides are conjugated to the surface of the disclosed devices. In various aspects, the cell-specific binding peptides with a loop or linear conformation may exhibit differential endothelial cell affinity on the modified Vaso-Locks.

In various additional aspects, polysaccharides are conjugated to the surface of the disclosed devices to impart anticoagulant properties. Non-limiting examples of suitable anticoagulant polysaccharides include cellulose, methylcellulose, hydroxylmethylcellulose, hydroxypropyl cellulose, hydroxypropylmethylcellulose, carboxymethylcellulose, sodium carboxymethylcellulose, cellulose sulfate, chitosan, chitosan salts, hyaluronic acid, hyaluronic acid salts, dextran sulfate, chondroitin sulfate, heparin, starch, and their derivatives.

As described in the examples herein, the anastomosis and anti-thrombogenic efficacies of the disclosed anastomosis devices have been evaluated in a swine carotid-jugular arteriovenous loop model.

In various aspects, the disclosed surface-modification approach facilitates endothelial cell affinity and anticoagulant abilities of the disclosed devices to provide long-term patency of the anastomosis. In some aspects, plasma treatment is used to modify the polymer surface of the disclosed device with specific chemical groups, including, but not limited to, amine or carboxylic acids. Through these chemical groups, we are able to further conjugate peptides, growth factors, or polysaccharides to these modified polymers. In various aspects, the peptides are selected from a synthesized library of peptide sequences with a loop or linear structures configured to facilitate endothelial cell affinity. Non-limiting examples of suitable sequences include the sequences summarized in Table 1. In some aspects, the peptides comprising SEQ ID NOS:1-2 and RGD are fibronectin-derived, and SEQ ID NOS: 3-5 are laminin-derived. In other aspects, the peptides comprising SEQ ID NOS: 6-28 have a looped structure and the peptides comprising SEQ ID NOS: 29-51 have a linear structure.

TABLE 1
Cell-Specific Binding Peptide Sequences.

| SEQ | |
|---|---|
| ID | |
| NO | PEPTIDE SEQUENCE |
| 1 | REDV |
| 2 | HGGVRLY |
| | RGD |
| 3 | IKVAV |
| 4 | PDSGR |
| 5 | YIGSR |
| 6 | CCRRGDWLC |
| 7 | CCRR KLDAPTWLC |
| 8 | CCRR PHSRNWLC |
| 9 | CCREDVC |
| 10 | CCHGGVRLYC |
| 11 | CCRRIKVAVWLC |
| 12 | CCRRYIGSRWLC |
| 13 | CCRRPDSGRWLC |
| 14 | CCLNSSQPSC |
| 15 | CCRRYVVLPRWLC |
| 16 | CCRR LDVPSWLC |
| 17 | CCRRIDAPSWLC |
| 18 | CCRREILDVPSTWLC |
| 19 | CCRR EDGIHELWLC |
| 20 | CCRRKAFDITYVRLKFWLC |
| 21 | CCRR FRHRNRKGYWLC |
| 22 | CCRR KRLDGSVWLC |
| 23 | CCRRFRHRNRKGYWLC |
| 24 | CCRRGQVFHVAYVLIKFWLC |
| 25 | CCRRGTNNWWQSPSIQNWLC |
| 26 | CCRRLWVTVRSQQRGLFWLC |
| 27 | CCRRWVTVTLDLRQVFQWLC |
| 28 | CCRRVLIKGGRARKHVWLC |
| 29 | CRGD |
| 30 | CKLDAPT |
| 31 | C PHSRN |
| 32 | CREDV |
| 33 | CHGGVRLY |
| 34 | CIKVAV |
| 35 | CYIGSR |
| 36 | CPDSGR |
| 37 | CLNSSQPS |
| 38 | CRYVVLPR |
| 39 | CLDVPS |
| 40 | CIDAPS |
| 41 | CEILDVPST |
| 42 | CEDGIHEL |
| 43 | CKAFDITYVRLKF |
| 44 | CFRHRNRKGY |
| 45 | CKRLDGSV |
| 46 | CFRHRNRKGY |
| 47 | CGQVFHVAYVLIKF |
| 48 | CGTNNWWQSPSIQN |
| 49 | CLWVTVRSQQRGLF |
| 50 | CWVTVTLDLRQVFQ |
| 51 | CVLIKGGRARKHV |

In some aspects, the disclosed devices may be surface-modified by the covalent attachment of anti-thrombolytic moieties including, but not limited to, heparin, antiplatelet agents, thrombolytic agents, and/or hydrophilic polymers.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1

To demonstrate the efficacy of the cell adhesion peptide RGD in promoting cell adhesion and endothelium formation, the following experiments were conducted.

The RGD peptide was incorporated into a hydrogel preparation and assessed in vitro in cell cultures, as well as in vivo using a rat model.

Materials

Thiolated HA, thiolated HA/heparin (HA-SH), and polyethylene glycol diacrylate [PEGDA, molecular weight (MW) 3.4 kDa] were purchased from Advanced BioMatrix, Inc. (Carlsbad, CA). Triethylamine (TEA), acryloyl chloride, 3-buten-1-ol, 1-ethyl-3-[3-(dimethylamino)propyl]carbodiimide (EDC), 3,3′-dithiobis(propanoic acid) (DTP), N,N-dimethylaminopyridine (DMAP), t-butyl methyl ether, 2,2-diphenyl-1-picrylhydrazyl (DPPH), and Pluronic F127 (MW 12.6 kDa) were obtained from Aldrich Chemical Co. (Milwaukee, WI). Dichloromethane (DCM) was purchased from Alfa Aesar (Ward Hill, MA). All other chemical reagents were obtained from Sigma-Aldrich (St. Louis, MO). The cell adhesion peptide, CCRRGDWLC, was synthesized by Nanjing Peptide Biotech Ltd. (Nanjing, China).

Synthesis of F127-SS-DA and F127-DA

The HA cross-linkers F127-DA and F127-SS-DA were first synthesized as reported previously. In brief, F127 was dissolved in DCM, followed by the addition of TEA. Acryloyl chloride was added to the mixture and stirred for 24 h at room temperature. The solution was poured into t-butyl methyl ether to precipitate the diacrylate product and collected by filtration. The product was then dissolved in water, dialyzed, and lyophilized to obtain F127-DA. As for F127-SS-DA, F127 was dissolved in DCM, followed by the addition of EDC and DMAP. DTP was added to the mixture and stirred for 24 h at room temperature. The mixture was precipitated in t-butyl methyl ether and further collected by filtration. The product was then dissolved in water, dialyzed, and lyophilized to obtain the intermediate SS-F127-SS. Then, SS-F127-SS was further dissolved in DCM, followed by the addition of EDC and DMAP. Then, 3-buten-1-ol was added and stirred for 24 h at room temperature. The mixture was purified as described above to obtain the final product F127-SS-DA. The structures of F127-DA, SS-F127-SS, and F127-SS-DA were confirmed by the Bruker AVANCE-III HD 500 MHz NMR spectrometer as we reported before.

Rheological properties of the HA hydrogels with F127-DA or F127-SS-DA were measured using a rheometer (HR-2, TA Instruments). HA-SH solution was fixed at 4 mg/mL. F127-DA or F127-SS-DA were 10, 20, and 50 mg/mL. Oscillation time sweep (1 h; 1% strain and 1 Hz) and strain sweep (0.1-10% strain and 1 Hz) were performed at 37° C. The hydrogel shear storage modulus (G′), loss modulus (G″), and gelation time at which G′ is equal to G″ was measured.

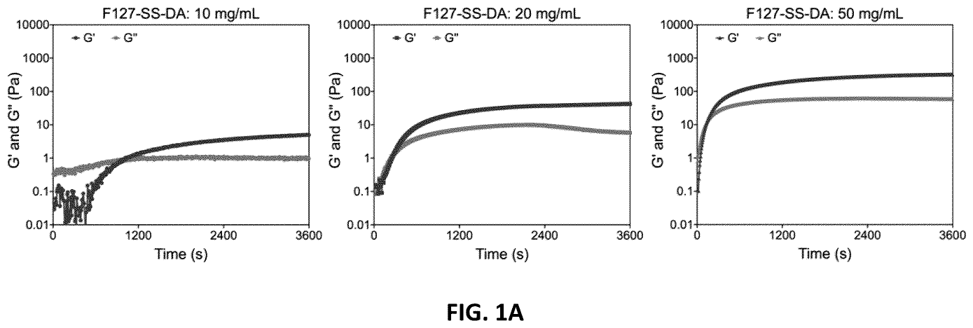

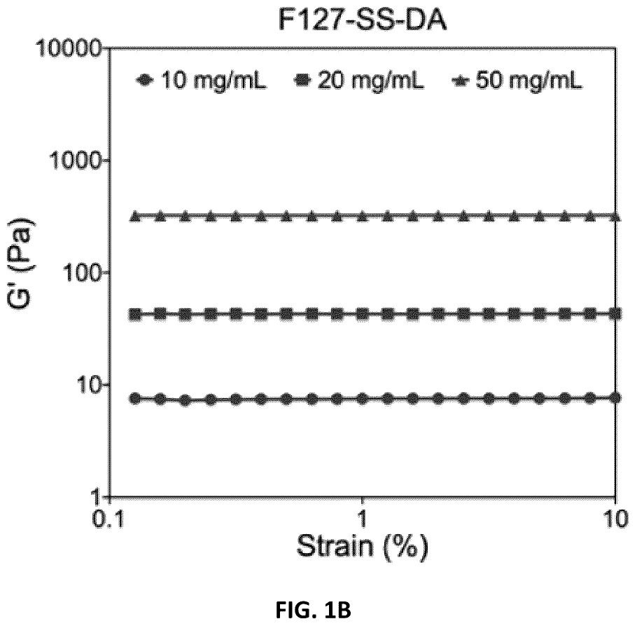

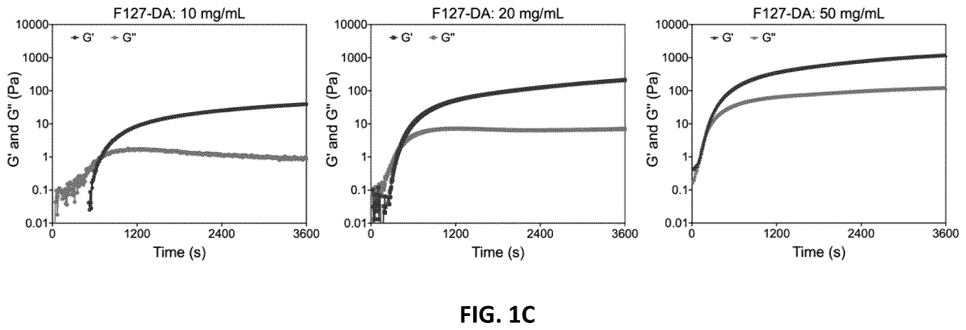

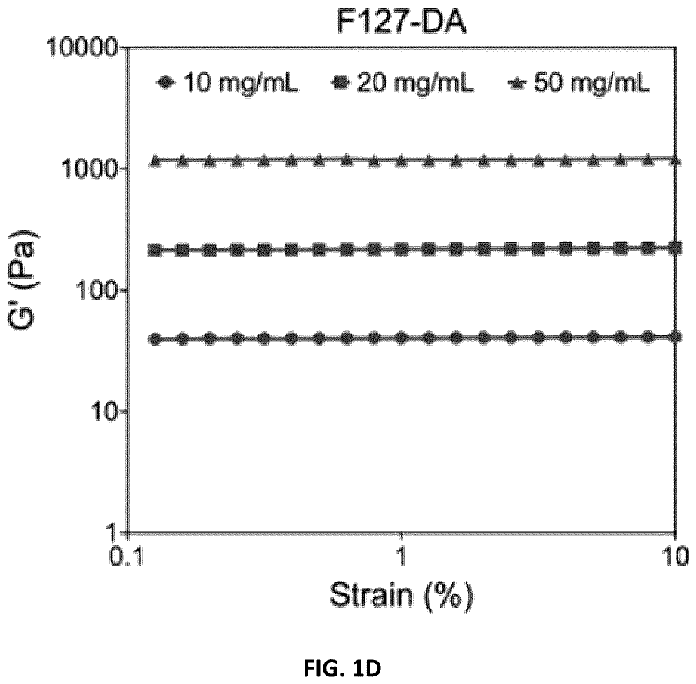

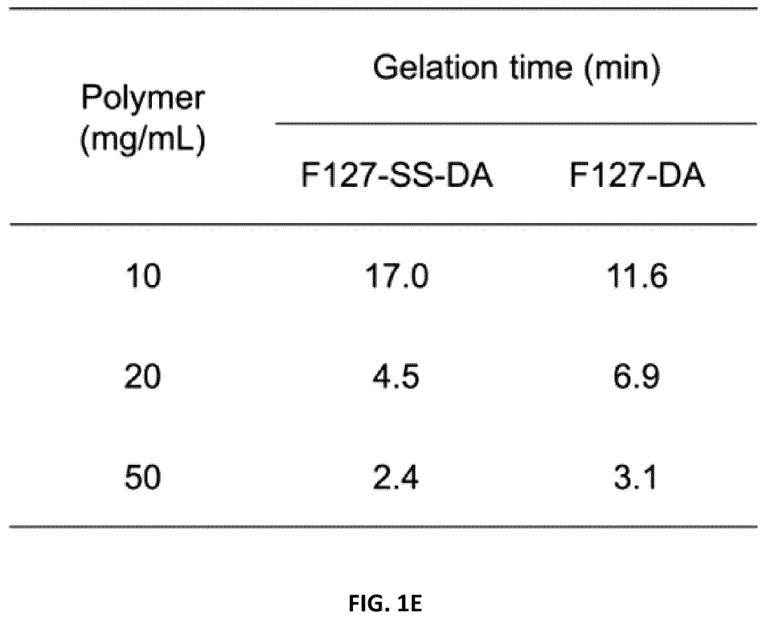

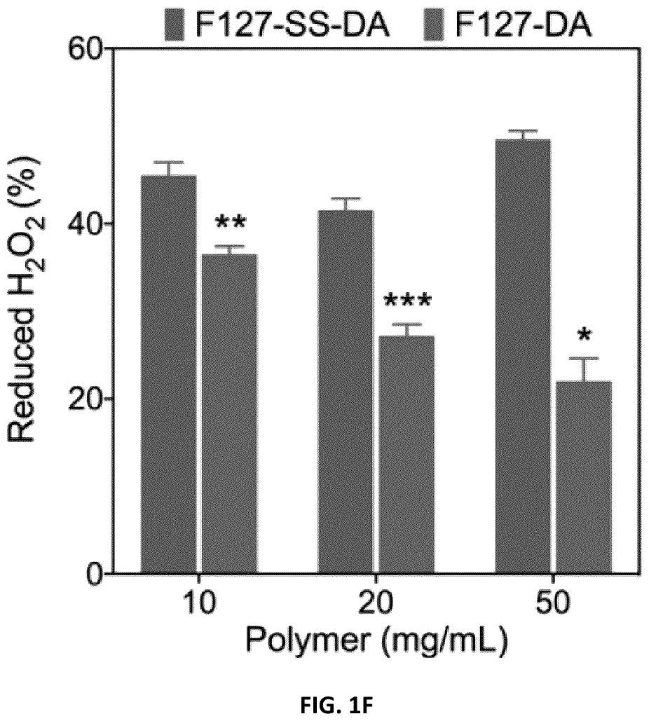

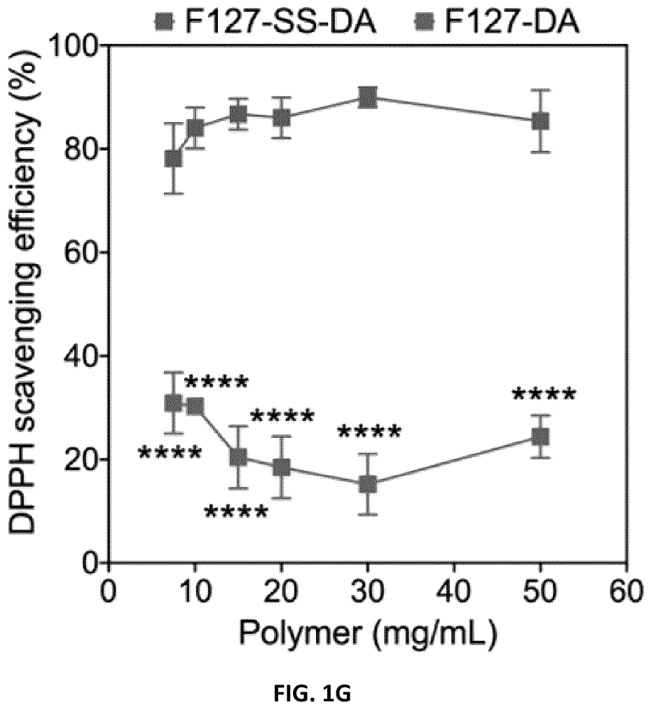

The cross-linkers F127-DA and F127-SS-DA were first synthesized as reported before. Both react with thiolated HA for hydrogel formation (FIGS. 1A, 1B, 1C, 1D, and 1E). The hydrogel formation is based on two types of reactions: (1) chemical cross-linking between diacrylates of F127-SS-DA or F127-DA and thiol groups of HA, which is the Michael-type addition reaction that can occur at physiological conditions without any additives or reaction byproducts; (2) physical cross-linking due to the thermosensitive behaviors of F127. An oscillatory time sweep was performed to check the hydrogel gelation time. The hydrogel gelation occurred at 2.4 min with 50 mg/mL F127-SS-DA and 3.1 min with 50 mg/mL F127-DA. The hydrogel gelation time with 20 mg/mL F127-SS-DA or F127-DA is 4.5 or 6.9 min, respectively. In contrast to F127-DA, faster gelation occurred in the hydrogels with F127-SS-DA at high concentrations (20 and 50 mg/mL). The disulfide bond in F127-SS-DA may act as a hydrophobic nucleus, which could interact with the hydrophobic polypropylene glycol, resulting in faster gelation.

Radical Scavenging Activities of HA Hydrogels

For the HA hydrogels, the HA-SH solution was fixed at 4 mg/mL and F127-DA or F127-SS-DA concentrations were 7.5, 10, 15, 20, 30, and 50 mg/mL, respectively. Hydrogels (50 μL) were added with DPPH (150 μL; 25 μM). The absorbance values at 517 nm were measured using a Multi-Mode Microplate Reader (Winooski, Vermont) and then the scavenging efficiency was calculated as reported before.

The Amplex red hydrogen peroxide peroxidase assay was adopted to investigate the consumption of H₂O₂ by HA hydrogels. These hydrogels were immersed in H₂O₂ (500 μL; 0.5 mM) for 24 h. The consumed H₂O₂ was calculated following the instruction of the assay.

The antioxidative capacity of HA hydrogels was evaluated next. F127-SS-DA-cross-linked HA hydrogels consumed significantly more H₂O₂ than those with the same concentration of F127-DA (FIG. 1F; 10 mg/mL: 41.6% vs 27.2%, P<0.01; 20 mg/mL: 45.5% vs 36.5%, P<0.001; 50 mg/mL: 49.7% vs 22.0%, P<0.05). Furthermore, independent of cross-linker concentrations, HA hydrogels with F127-SS-DA displayed significantly higher scavenger effects for DPPH (FIG. 1G; 7.5 mg/mL: 78.1% vs 30.9%, P<0.0001; 10 mg/mL: 84.1% vs 30.2%, P<0.0001; 15 mg/mL: 86.7% vs 20.4%, P<0.0001; 20 mg/mL: 86.0% vs 18.5%, P<0.0001; 30 mg/mL: 90.0% vs 15.2%, P<0.0001; 50 mg/mL: 85.4% vs 24.4%, P<0.0001). Collectively, these data confirm the antioxidative effects of F127-SS-DA-cross-linked HA hydrogels.

Cell Culture

Two types of cells, human umbilical vein endothelial cells (HUVECs) and human adipose-derived stem cells (hASCs) were obtained from Lonza (Alpharetta, GA). Both cells were cultured according to the provided protocols and used before passage 5 in this study.

Angiogenesis and vascularization assays were adopted to optimize hydrogels with RGD. For the two-dimensional (2D) culture of HUVECs, cells (4×10⁴/well in a 96-well plate) were plated on the surface of HA hydrogels (40 μL; HA-SH: 4 mg/mL and PEGDA: 5 mg/mL) with various concentrations of RGD (0, 0.01, 0.1, and 1 mM). Matrigel was used as a positive control. After 24 h, HUVECs were imaged after LIVE/DEAD staining. As for the 3D cell culture, to avoid cell precipitation onto the surface of the plate, the plate was first coated with the hydrogels (40 μL hydrogel; HA-SH: 4 mg/mL, PEGDA: 2 mg/mL, F127-SS-DA: 4 mg/mL; RGD: 0, 0.10, 0.25, and 0.50 mM). Then, HUVECs and hASCs mixed with hydrogel precursor solutions (0.75×10⁶/mL hydrogel; 40 μL hydrogel) were plated on top of hydrogels. In addition, both cells were cultured inside hydrogels with F127-SS-DA or F127-DA (40 μL; HA-SH: 4 mg/mL, PEGDA: 2 mg/mL, RGD: 0.1 mM; F127-DA or F127-SS-DA: 2, 4, 8, 10, 20, and 50 mg/mL). The cells were imaged after immunostaining for F-actin. The Angiogenesis Analyzer from ImageJ software was applied to evaluate vascular-like network formation in terms of the branch number, total length of branches, and mean mesh size following the instruction of the Angiogenesis Analyzer.

Cell viability was examined with a LIVE/DEAD staining kit and then the images were captured by a Zeiss LSM 880 laser scanning confocal microscope. The cell morphology was also evaluated by immunostaining with Alexa Fluor Phalloidin 568 or 488 for F-actin and 4′,6-diamidino-2-phenylindole dihydrochloride (DAPI, Molecular Probes) for cell nuclei.

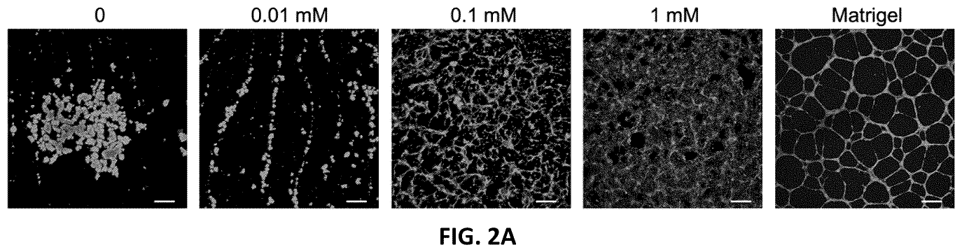

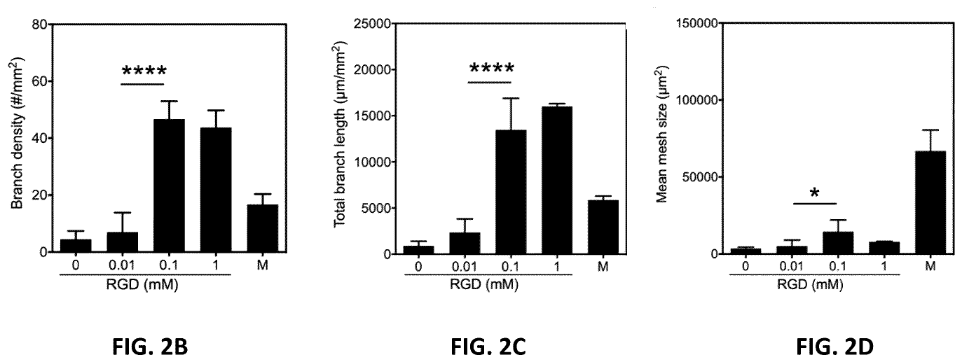

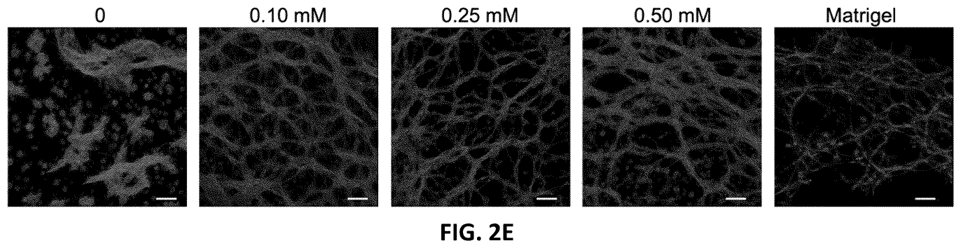

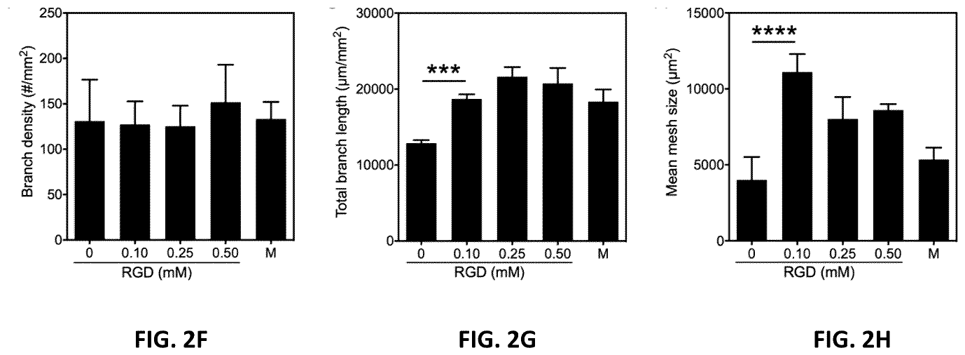

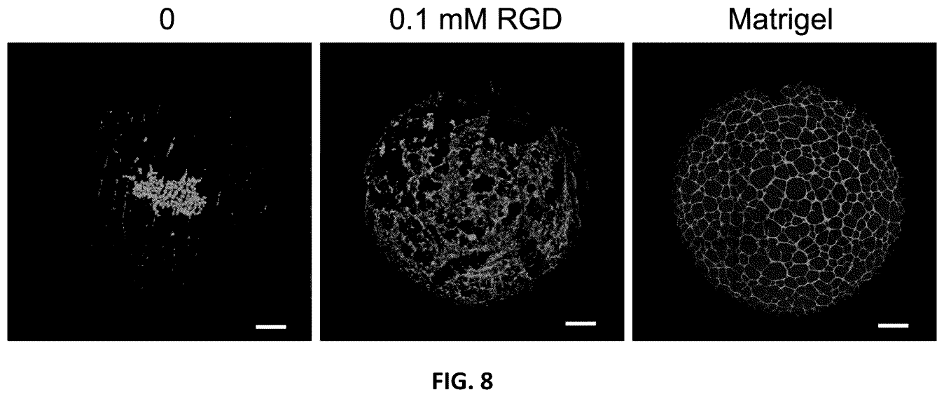

HA hydrogels were optimized to support cell attachment and organization under both 2D and 3D conditions. First, various amounts of RGD (0, 0.01, 0.1, and 1 mM) were conjugated with HA hydrogels, and their effects on vascular-like network formation were evaluated by an in vitro Matrigel angiogenesis assay (FIGS. 2A and 8). Without RGD, HUVECs formed clusters on the surface of the HA hydrogel without spreading. Similar cell clusters also assembled on the hydrogel with 0.01 mM RGD. In contrast, on the hydrogel with 0.1 mM RGD, HUVECs displayed higher densities of vascular-like branches (FIG. 2B; 47/mm² vs 7/mm², P<0.0001), longer branch length (FIG. 2C; 13,473 vs 2361 μm/mm², P<0.0001), and larger mesh size (FIG. 2D; 14,422 vs 5163 μm², P<0.05). There was no significant difference between the hydrogel with 0.1 mM and that with 1 mM RGD. When compared to those on the surface of Matrigel, cells on these two hydrogels (0.1 and 1 mM RGD) showed a smaller mesh size (FIG. 2D; 14,422 μm² vs 7873 μm² vs 66,900 μm², P<0.0001). Taken together, the HA hydrogel with 0.1 mM RGD supports HUVECs to form vascular-like networks (FIG. 8).

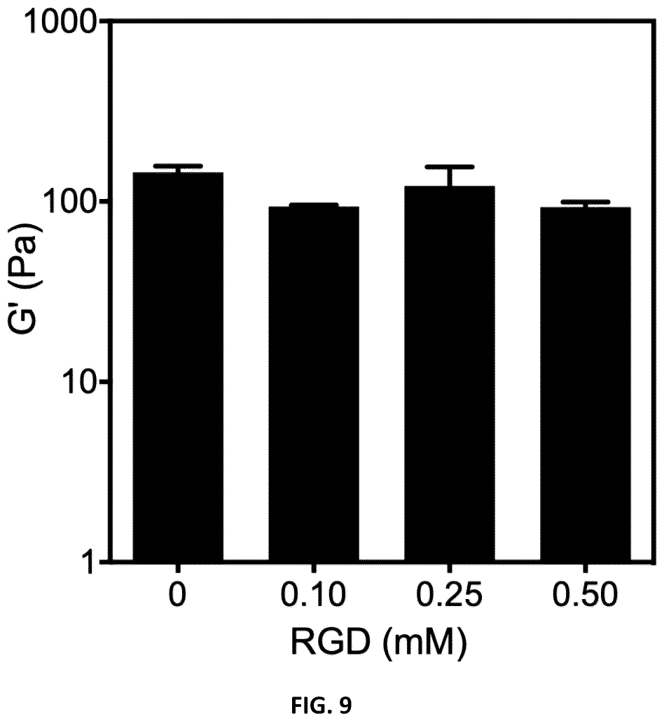

To further evaluate cell organization inside the HA hydrogels in 3D, an established in vitro vascularization model was adopted. hASCs and HUVECs were co-cultured inside HA hydrogels with different amounts of RGD (0, 0.10, 0.25, and 0.50 mM). These cells resulted in 3D vascular-like morphogenesis with the formation of multicellular tubular structures (FIG. 2E). In particular, compared to the plain hydrogel (without RGD), the development of vascular-like structures was more pronounced in the hydrogels with the RGD concentration as low as 0.10 mM with longer total branch length (FIG. 2G; 18,712 vs 12,898 μm/mm², P<0.001) and larger mesh size (FIG. 2H; 11,106 vs 4006 μm², P<0.0001). All of the hydrogels containing RGD (0.10, 0.25, and 0.50 mM) promoted the development of 3D tubular networks reminiscent of capillaries. RGD conjugation did not affect the stiffness of these hydrogels, all of which had G′ around 100 Pa (FIG. 9). Taken together, these data demonstrate that the HA hydrogel with 0.1 mM RGD supports vascular-like network formation in both 2D and 3D conditions.

Hydrogel-Mediated Cell Protection against H2O2-Induced Oxidative Damage

HUVECs and hASCs were cultured inside hydrogels (40 μL; HA-SH: 4 mg/mL, PEGDA: 2 mg/mL, RGD: 0.10 mM, F127-SS-DA or F127-DA: 4 mg/mL; 0.75×106/mL hydrogel) in the medium with various concentrations of H2O2 (0, 1.0, 2.5, and 5.0 mM) for 24 h. The vascular-like networks inside the hydrogels were evaluated as described above to examine the ability of HA hydrogels against oxidative damage. In addition, the intracellular ROS levels were measured by a ROS assay kit (ab113851, Abcam) and the fluorescence intensity (excitation/emission at 485 nm/535 nm) was qualified using the microplate reader.

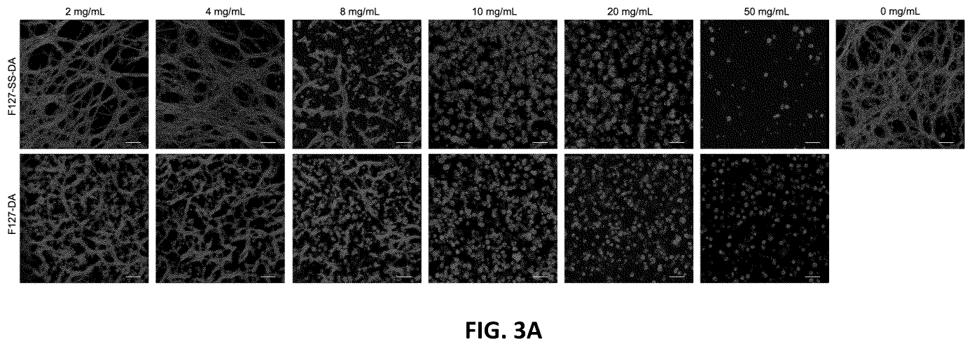

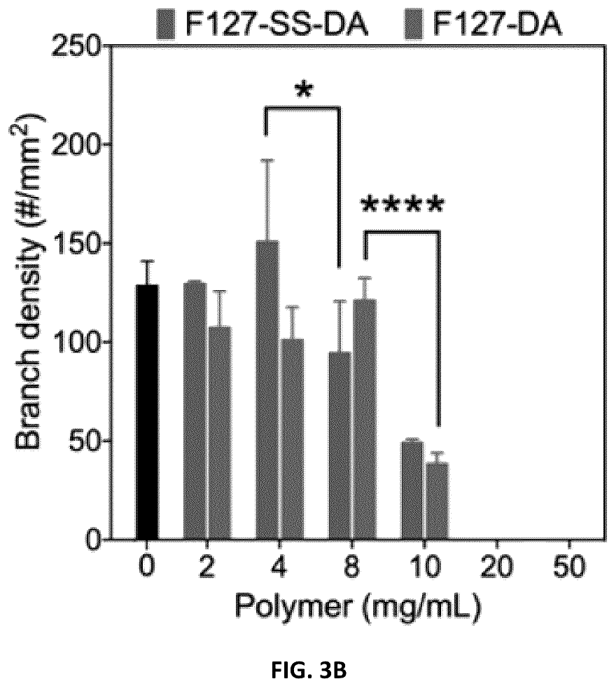

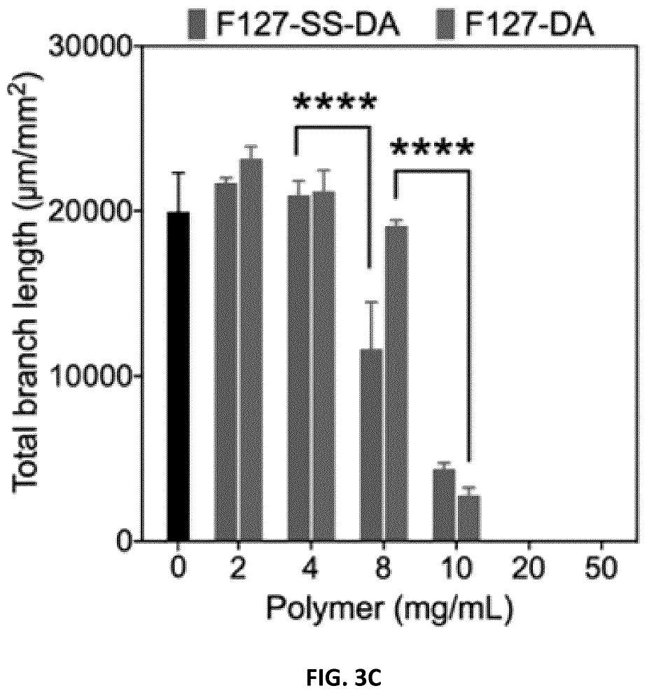

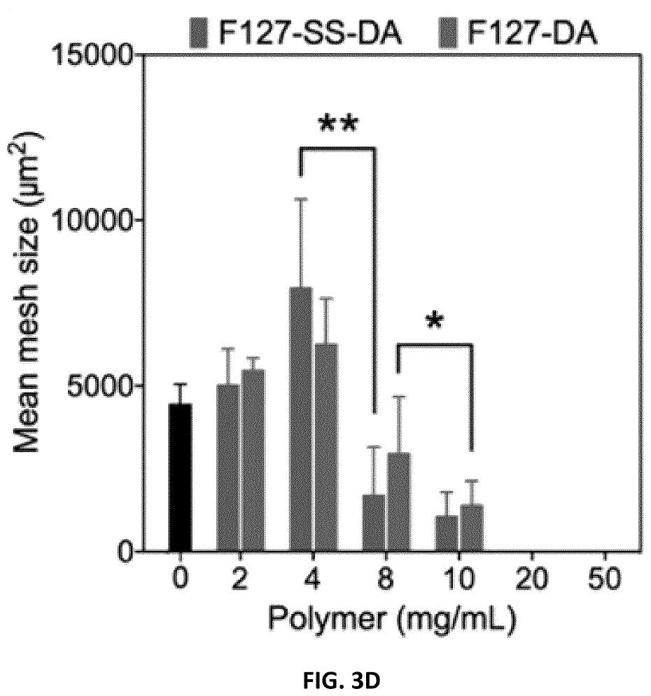

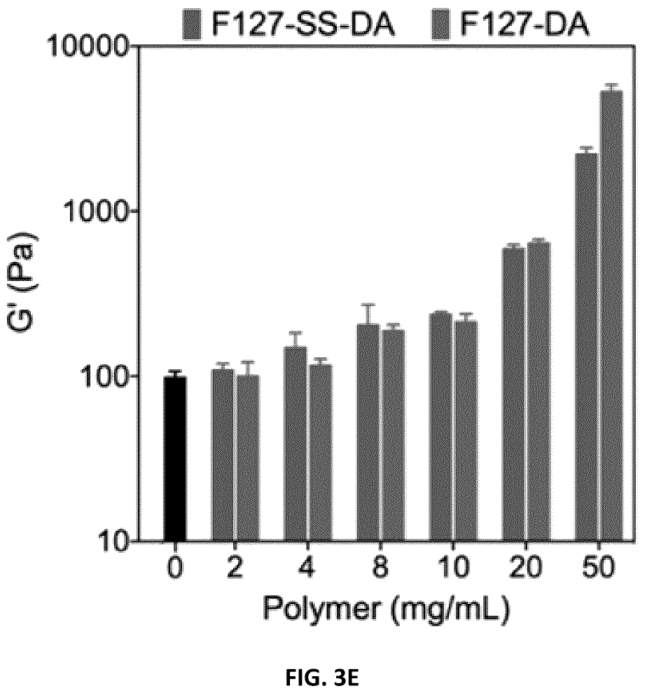

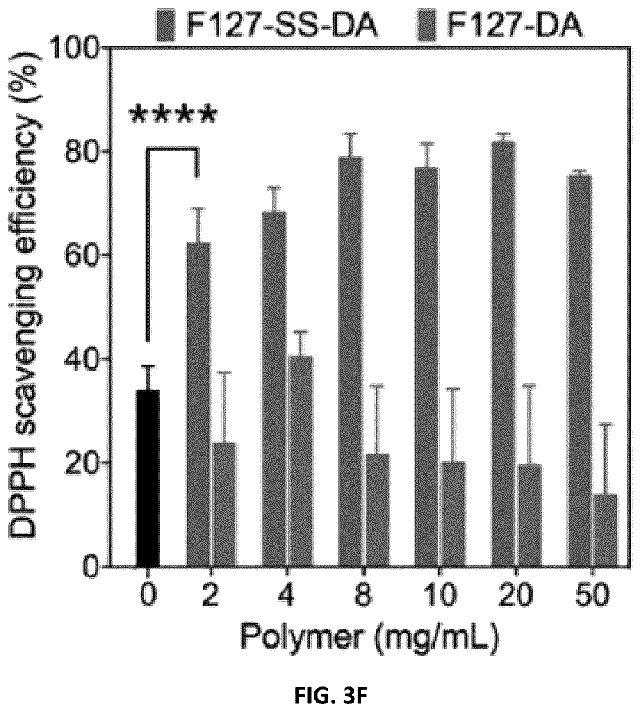

In the following experiments, all HA hydrogels had 0.1 mM RGD. Further, the effects of adding the antioxidative component F127-SS-DA on the formation of a vascular-like network in 3D were tested. First, cell organization was examined inside HA hydrogels with F127-SS-DA or F127-DA (2, 4, 8, 10, 20, and 50 mg/mL). Inside the hydrogels with F127-SS-DA of 2 or 4 mg/mL, cells formed vascular-like networks in 3D (FIG. 3A). Compared to that with 8 mg/mL, the hydrogel with 4 mg/mL F127-SS-DA has shown significantly higher densities of vascular-like branches (FIG. 3B; 151/mm² vs 95/mm², P<0.05), longer total branch length (FIG. 3C; 20,964 vs 11,638 μm/mm², P<0.0001), and larger mesh size (FIG. 3D; 7986 vs 1728 μm², P<0.01). Inside the hydrogels with more than 8 mg/mL F127-SS-DA, cells could not form vascular-like networks. In particular, cells could not spread inside hydrogels with F127-SS-DA of 20 or 50 mg/mL. In parallel, inside the hydrogels with less than 8 mg/mL F127-DA, cells formed vascular-like networks (FIG. 3A). When compared to that with 10 mg/mL F127-DA, these cells have shown significantly higher densities of vascular-like branches (FIG. 3B; 122/mm² vs 39/mm², P<0.0001), longer total branch length (FIG. 3C; 19,111 vs 2775 μm/mm², P<0.0001), and larger mesh size (FIG. 3D; 5158 vs 2892 μm², P<0.05). Inside the hydrogels with more than 8 mg/mL F127-DA, cells formed clusters instead of vascular-like networks.

The stiffness of hydrogels increased with more F127-SS-DA or F127-DA. When the stiffness of hydrogel surpassed 200 Pa (FIG. 3E; F127-SS-DA or F127-DA>8 mg/mL), cells could not form vascular-like networks. The DPPH assay was also used to assess the antioxidative abilities of these hydrogels. Interestingly, even with 2 mg/mL F127-SS-DA, the hydrogel has shown significant DPPH scavenging efficiency compared to that without F127-SS-DA (FIG. 3F; 62.5% vs 29.1%, P<0.0001). All hydrogels with F127-SS-DA have shown higher scavenging efficiencies (over 60%) than those with the same concentration of F127-DA.

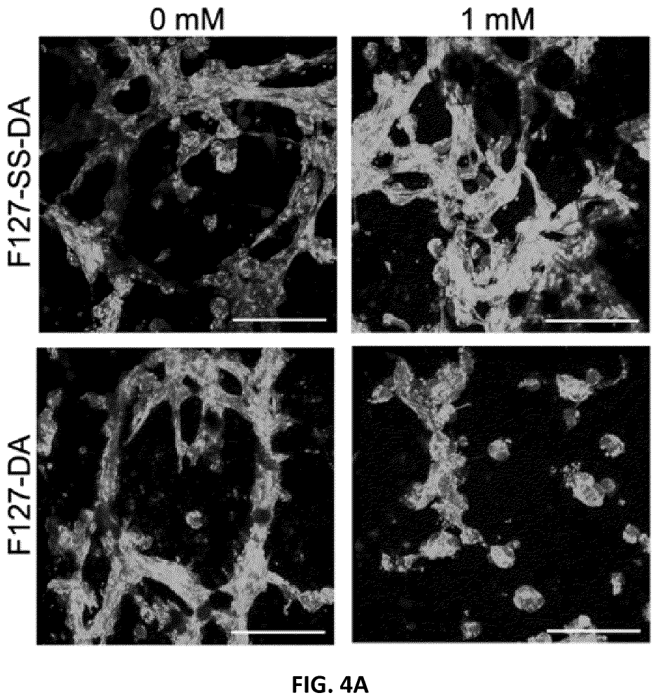

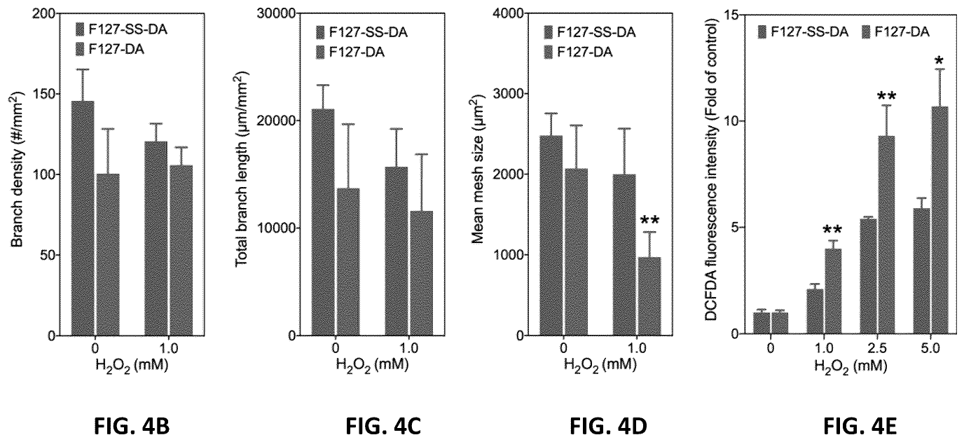

Second, whether or not the HA hydrogels could preserve vascular-like networks under H₂O₂ conditions was evaluated (FIG. 4A). When exposed to 1 mM H2O2, the vascular-like networks inside the HA hydrogel with F127-SS-DA were stable with a larger mesh size than those inside the hydrogel with F127-DA (FIG. 4D; 15,692 vs 11,604 μm², P<0.01). Additionally, the ROS level in cells in response to H₂O₂ was investigated. Cells in the HA hydrogel with F127-SS-DA compared to those inside the hydrogel with F127-DA have shown lower intracellular ROS levels under H₂O₂ conditions (FIG. 4E; 1.0 mM: 2.1-fold vs 4.0-fold, P<0.01; 2.5 mM: 5.4-fold vs 9.3-fold, P<0.01; 5.0 mM: 5.9-fold vs 10.7-fold, P<0.05). All of these results demonstrate that our optimal hydrogel with F127-SS-DA not only supports vascular-like networks in 3D but also preserves these networks against H2O2-induced damage.

VEGF Release from HA Hydrogels

The ability of HA hydrogels for long-term release of the VEGF was evaluated as reported before. As for HA hydrogels, HA-SH solution (with or without thiolated heparin) was fixed at 4 mg/mL and F127-SS-DA was 50 mg/mL. Hydrogels (100 μL) were loaded with 1 μg of the VEGF and the release amount was quantified with a VEGF ELISA kit (Peprotech, Cranbury, NJ).

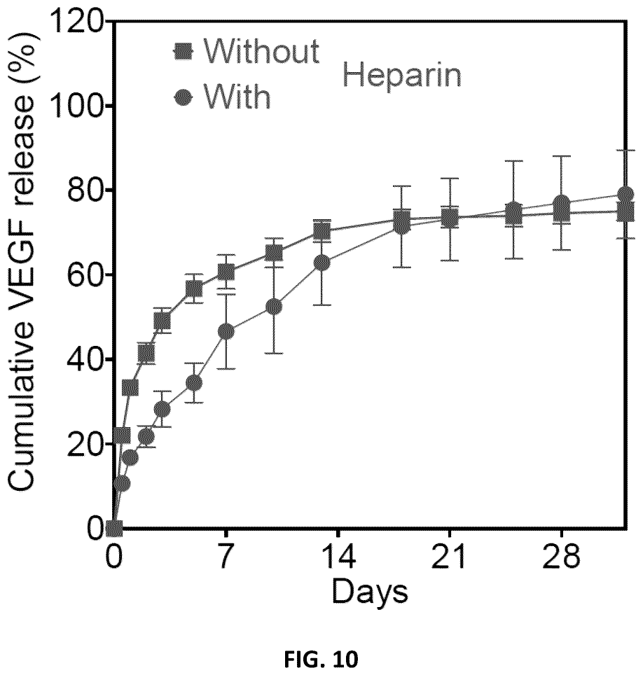

First, the ability of HA hydrogels to release growth factors for the purpose of cell recruitment was examined. Heparin has been widely used in hydrogels to help preserve the bioactivity of growth factors, delay their release, and protect them from degradation by proteinases (FIG. 10). Thiol-modified heparin was applied into HA hydrogels and then the VEGF was loaded. With heparin, HA hydrogels provided a much steadier release of the VEGF during the first 14 days (FIG. 10). HA hydrogels with F127-SS-DA (50 mg/mL) provided sustained release of the VEGF with a cumulative amount of 75.0% at 4 weeks (FIG. 10).

Femoral Artery Ligation Model and Hydrogel Injection

Hindlimb ischemia was induced in a rat model through ligating and excising of the femoral arteries. Rats were first anesthetized using a 2-3% isoflurane/oxygen mixture. The right femoral artery was exposed from a level just distal to the inguinal ligament to its bifurcation to saphenous and popliteal arteries using a longitudinal incision. The femoral artery and its side branches were then dissected free, ligated proximally and distally, and excised. Seven days after ligation, hydrogels (HA-SH: 4 mg/mL; F127-SS-DA: 50 mg/mL; 0.1 mM RGD) with or without the VEGF (1 μg/rat) were intramuscularly injected into the triceps surae muscles of the ischemic hindlimb. The injection volume was 160 μL (20 μL/point) at separate locations (rostral, middle, and caudal). The animal protocol has been approved by the Animal Care and Use Committee of UNMC (ID: 19-078-07-FC).

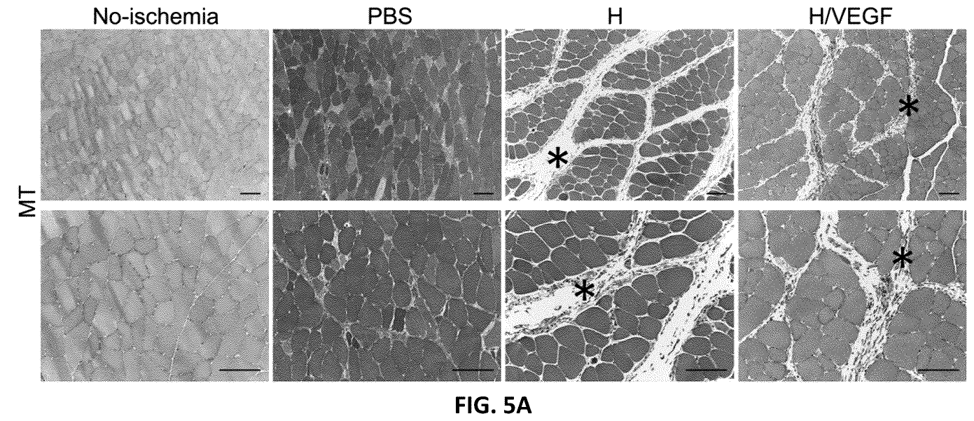

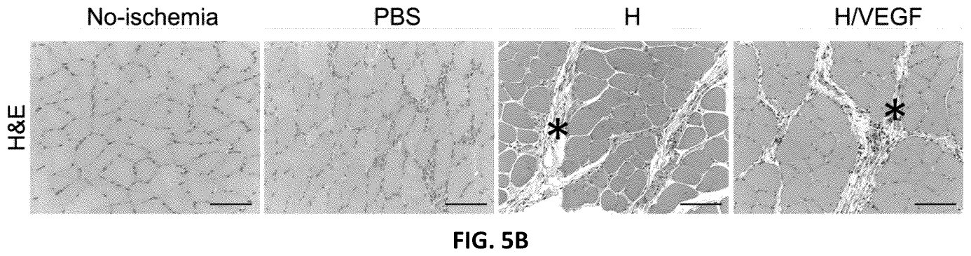

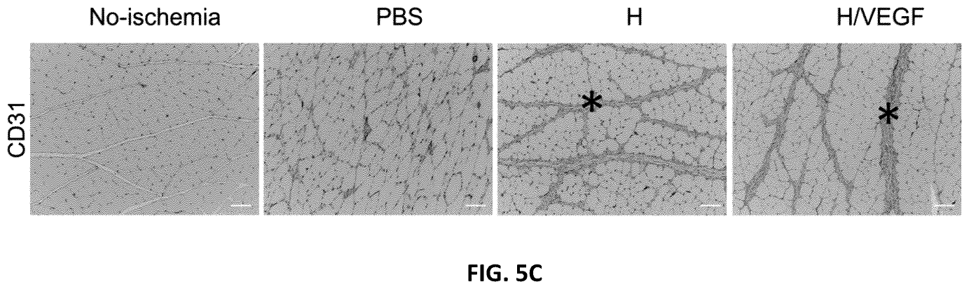

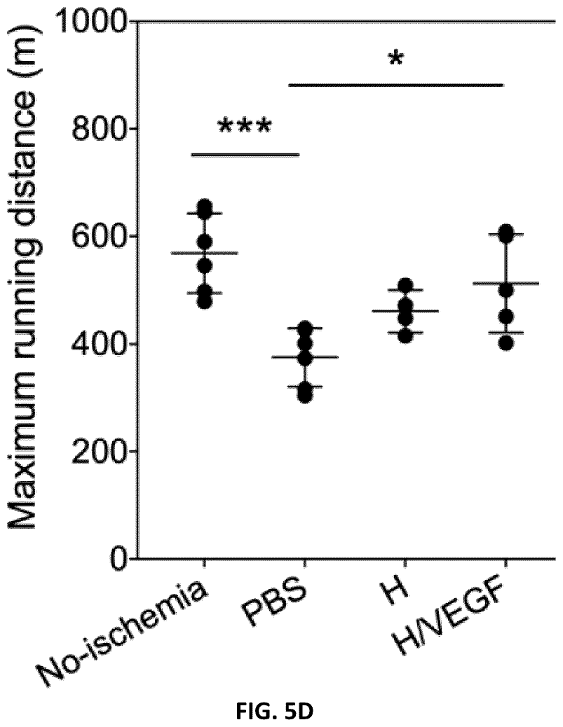

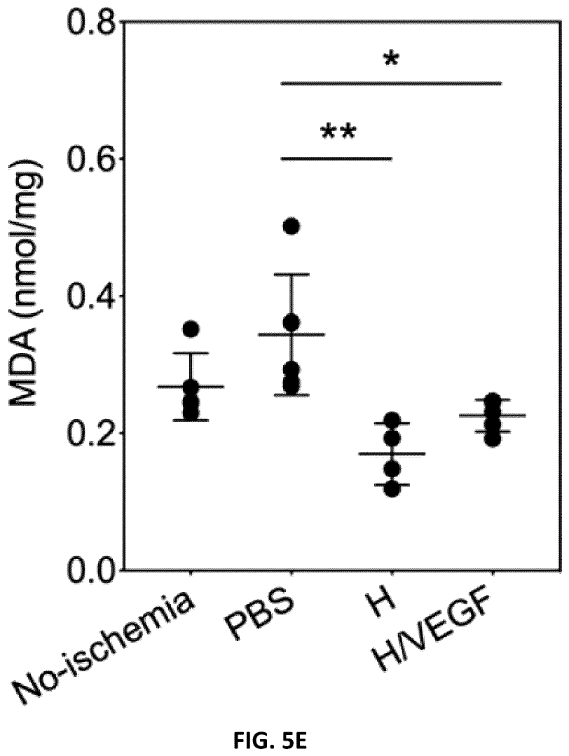

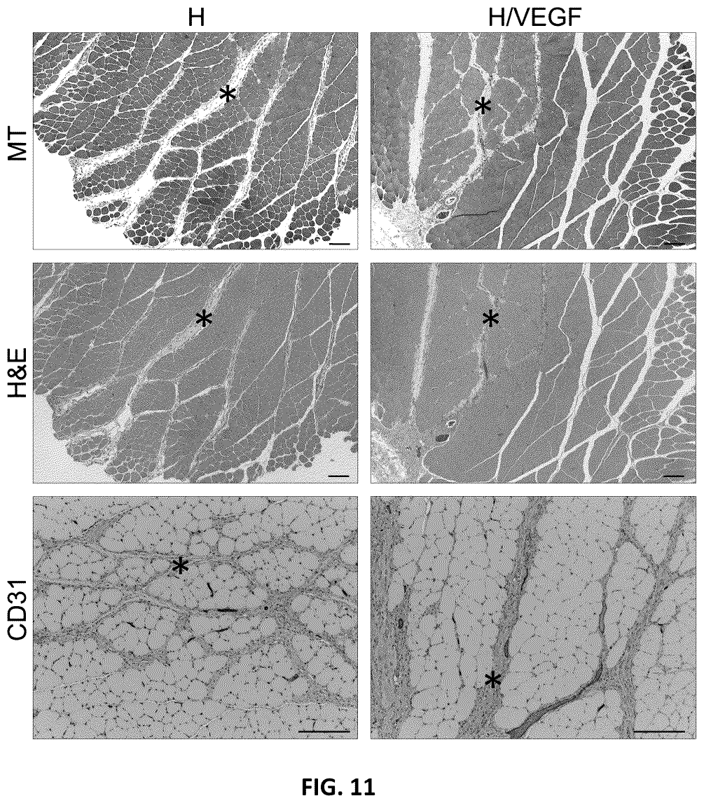

The effects of HA hydrogels with or without the VEGF on the functional recovery of rats with hindlimb ischemia were then examined. A femoral artery ligation/excision model was adopted to produce hindlimb ischemia and mimic PAD in rats. Histology was performed to evaluate the biocompatibility of hydrogels in vivo. The injected hydrogels were identified between myofascicles (FIGS. 5 and 11). While muscles treated with hydrogels demonstrated increased distribution of myofiber shape and centrally located nuclei with minimal fibrosis (FIGS. 5A and 5B), the gastrocnemius muscle of legs treated with phosphate-buffered saline (PBS) demonstrated multiple degenerated, atrophic myofibers and moderate endomysial and perimysial fibrosis. The capillary density inside the gastrocnemius was further evaluated. Both hydrogels enhanced the capillary density compared to the PBS group (FIG. 5C).

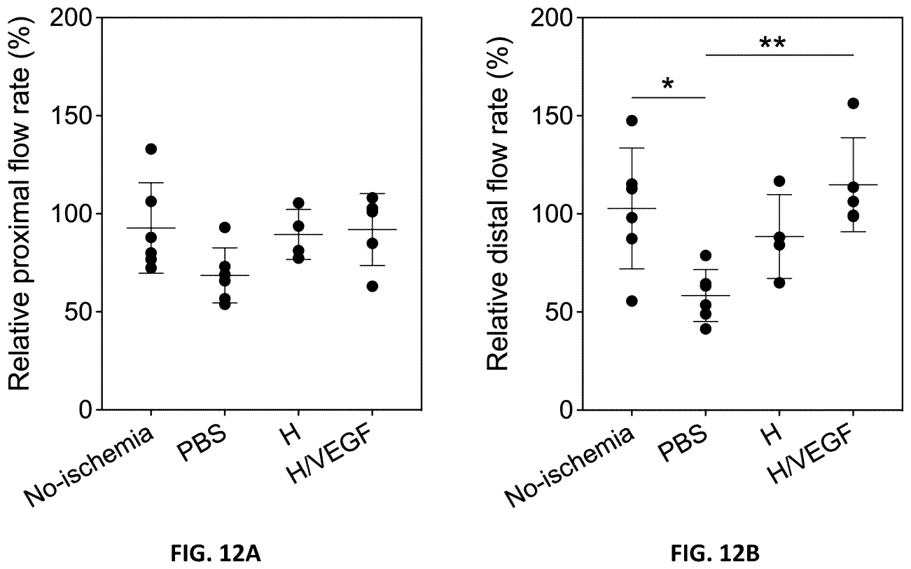

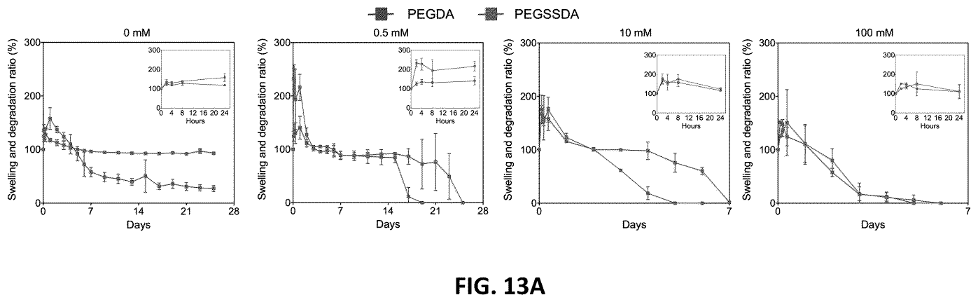

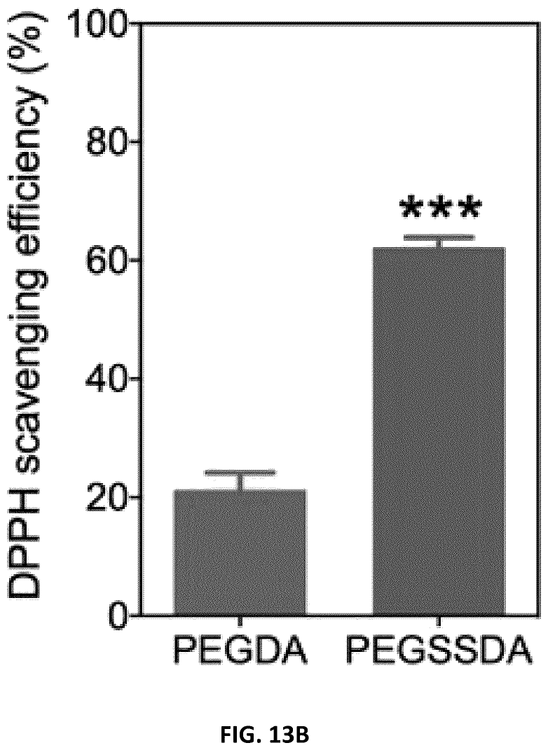

A laser Doppler probe was used to examine the velocity of blood flow inside the gastrocnemius. When treated with PBS, the rats with hindlimb ischemia showed significantly decreased blood flow at the distal site inside the gastrocnemius compared to the no-ischemia control (FIG. 12; 102.8% vs 58.4%, P<0.05). The HA hydrogel with the VEGF produced significantly improved blood flow compared to the PBS group (FIG. 12; 114.8% vs 58.4%, P<0.01).

Treadmill Running Test

The ambulatory performance of our rats was examined by using treadmill running tests 6 weeks after the hydrogel injection. It was determined that the maximum running distance with a motorized treadmill equipped with an electrical grid at the end of the lane. The exercise duration and distance were recorded beginning at a speed of 13 m/m in with an increase of 3 m/m in each 2 min until exhaustion. Exhaustion was defined as the failure of the rat to maintain its running speed, indicated by the hind feet making contact with the electrical grid 3 times within 10-second intervals.

The effects of HA hydrogels on the running performance of rats after hindlimb ischemia were further evaluated. Rats treated with PBS had a running performance deficit compared to the nonischemic control (FIG. 5D; 569.0 vs 374.9 m, P<0.001). The HA hydrogel without the VEGF increased the running distance, but the increase did not reach significance (FIG. 5D; 461.0 vs 374.9 m, P=0.25). In contrast, the HA hydrogel with the VEGF, when compared to the PBS group, produced significantly improved running distance (FIG. 5D; 512.6 vs 374.9 m, P<0.05).

Regional Blood Flow Examination

Regional blood flow velocity inside the gastrocnemius 6 weeks after the hydrogel injection was next examined by Transonic Tissue Perfusion Monitor BLF22 with a monofiber probe (Transonic Systems Inc., Ithaca, NY). The blood flow velocity is defined as 0-10 units proportional to the average flow velocity of moving red blood cells in m/s. The probe was placed in two sites for recording: (1) a proximal site is located around the inguinal ligament; (2) a distal site is at the middle distance between the knee and the start of the Achilles tendon. For each rat, the flow velocity in the ischemic muscle was first normalized to that of the nonischemic hindlimb and then compared among all the groups.

Histology

Histology was used to evaluate hydrogel retention inside the muscle 6 weeks after injection. Gastrocnemius muscles were dissected and fixed in 4% w/v paraformaldehyde overnight. Specimens were sectioned to a thickness of 5 μm using a microtome and stained with hematoxylin and eosin (H&E) and Masson's trichrome (MT) to visualize the injected hydrogels and evaluate their biocompatibility. Samples were imaged as above and at least six visual fields were randomly selected.

For immunostaining, CD31 (ab28364, Abcam) was incubated overnight at 4° C.; then, the biotinylated secondary antibody followed by the streptavidin-HRP was incubated at room temperature. In consideration of quantitative analysis, four random fields per sample were analyzed. The numbers of vessels present within the field were counted and compared among all groups.

Lipid Peroxidation Assay and Oxidative Stress PCR Array Assay

To examine the oxidative stress level in the ischemic hindlimb, malondialdehyde (MDA; ab118970, Abcam) was first measured inside the gastrocnemius muscle. An oxidative stress PCR array assay was also performed. RNA from the gastrocnemius (10 mg/rat) was isolated by an RNeasy Plus Mini Kit (QIAGEN) and then converted to cDNA by an RT2 PreAMP cDNA synthesis kit (QIAGEN). The rat oxidative stress RT2 PCR array (PARN-065Z, QIAGEN) was used to profile the oxidative stress-relative gene expression. The fold change was calculated by the RT2 Profiler PCR array data analysis software online.

To evaluate oxidative stress in the ischemic hindlimb, the level of MDA (produced by the reaction of ROS with polyunsaturated lipids) was first measured in the gastrocnemius. It was found that treatment with hydrogels with and without the VEGF produced significantly lower levels of MDA when compared to the PBS group (FIG. 5E; 0.22 vs 0.17 vs 0.32 nmol/mg muscle, P<0.05).

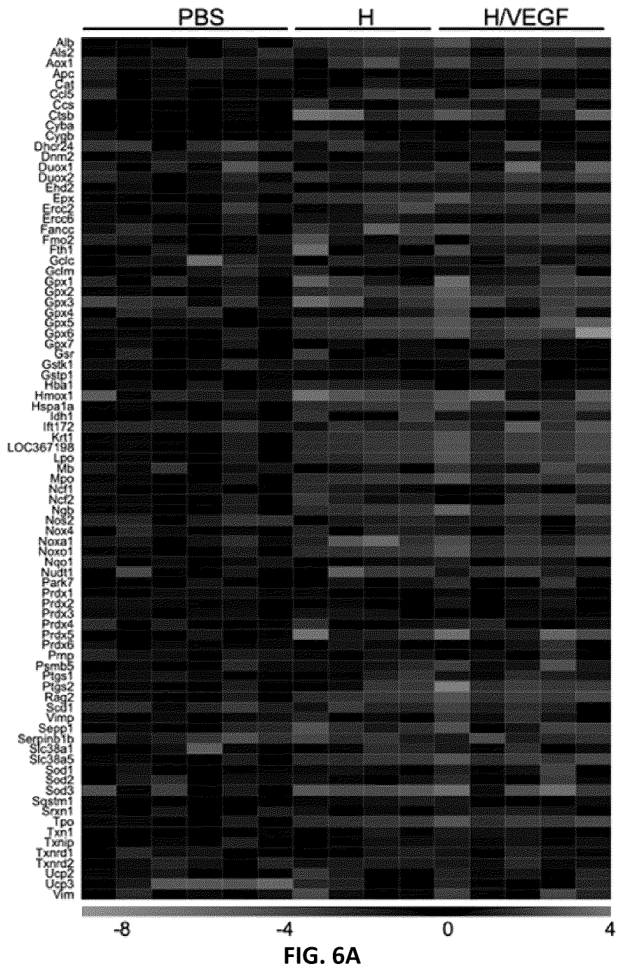

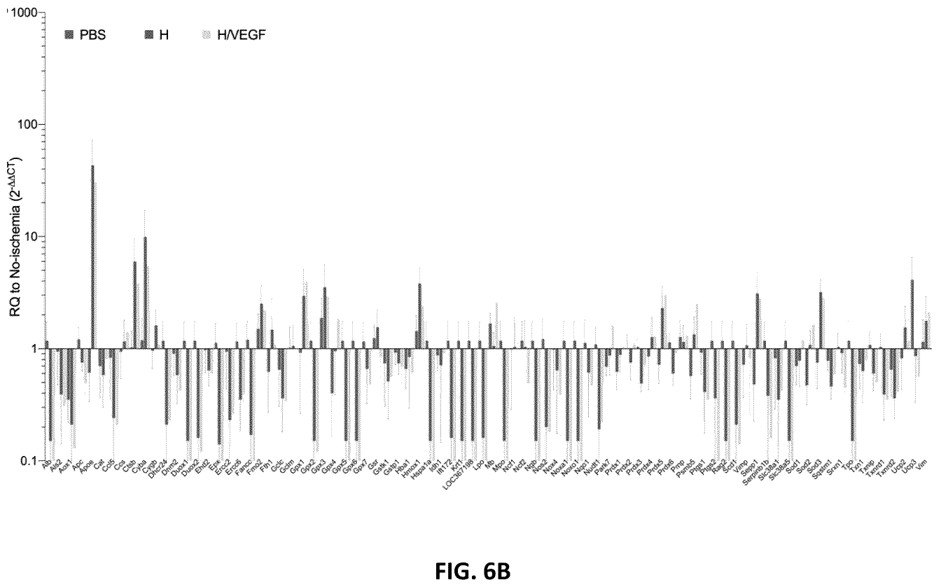

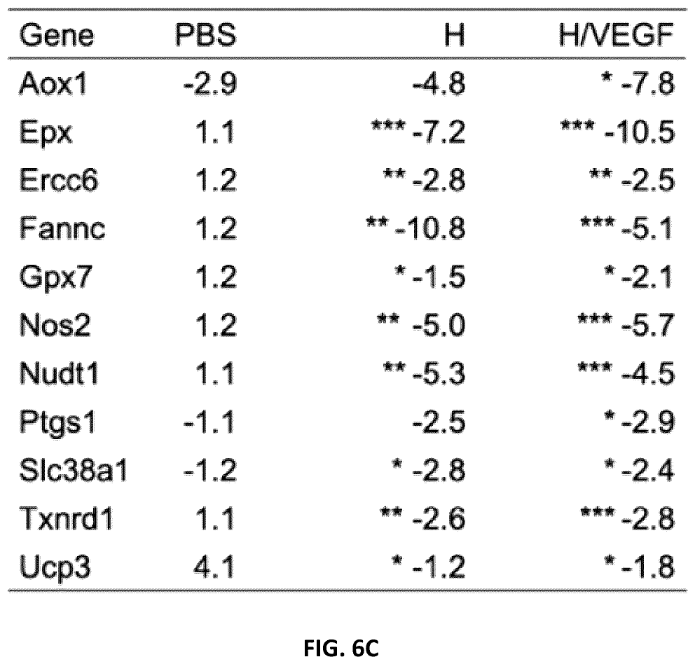

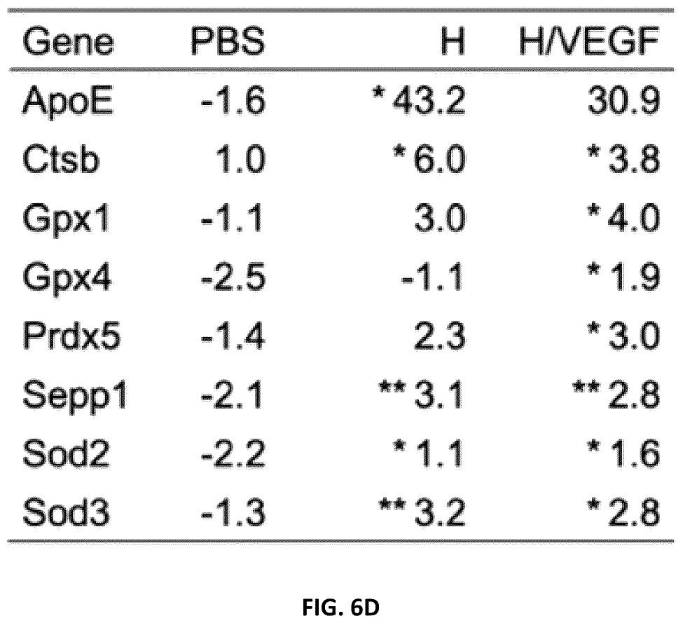

Oxidative stress-related gene expression inside the gastrocnemius was further studied using an oxidative stress PCR array assay (FIGS. 6A and 6B). A set of genes were found to be significantly downregulated by the HA hydrogels (FIG. 6C). For example, the expression of mitochondrial uncoupling protein 3 (Ucp3), which codes for a mitochondrial anion carrier, was significantly lower in the legs treated with the VEGF-containing hydrogel (FIG. 6C; Gene expression relative to nonischemic control; PBS: 4.1-fold; H: −1.2-fold, P<0.05; H/VEGF: −1.8-fold, P<0.05). Similarly, inducible nitric oxide synthase 2 (Nos2) was significantly downregulated in both hydrogel-treated groups (FIG. 6C; PBS: 1.2-fold; H: −5.0-fold, P<0.01; H/VEGF: −5.7-fold, P<0.001). Furthermore, the expression of nudix hydrolase 1, an oxidized purine nucleoside triphosphatase, was significantly lower in the ischemic hindlimbs of hydrogel-treated rats (FIG. 6C; PBS: 1.1-fold; H: −5.3-fold, P<0.01; H/VEGF: −4.5-fold, P<0.001). Other genes displaying downregulation in the legs treated with hydrogels included eosinophil peroxidase (Epx; FIG. 6C; PBS: 1.1-fold; H: −7.2-fold, P<0.001; H/VEGF: −10.5-fold, P<0.001) and Fanconi anemia complementation group C (FANNC; FIG. 6C; PBS: 1.2-fold; H: −10.8-fold, P<0.01; H/VEGF: −5.1-fold, P<0.001).

In comparison, several genes have been significantly upregulated by the hydrogels (FIG. 6D). For instance, apolipoprotein E (ApoE), which is essential for the normal metabolism of lipoproteins, was significantly highly expressed in the hydrogel groups compared to the PBS group (FIG. 6D; gene expression relative to nonischemic control; PBS: −1.4-fold; H: 43.2-fold, P<0.05; H/VEGF: 30.9-fold). Glutathione peroxidase 1 (Gpx1) was upregulated in the hydrogel groups (FIG. 6D; PBS: −1.1-fold; H: 3.0-fold; H/VEGF: 4.0-fold; P<0.05), as well as peroxiredoxines 5 (Prdx5; FIG. 6D; PBS: −1.4-fold; H: 2.3-fold; H/VEGF: 3.0-fold, P<0.05) and cathepsin B (Ctsb; FIG. 6D; PBS: 1.0-fold; H: 6.0-fold, P<0.05; H/VEGF: 3.8-fold, P<0.05). Other antioxidant genes, including selenoprotein P (Sepp1; FIG. 6D; PBS: −2.1-fold; H: 3.1-fold, P<0.01; H/VEGF: 2.8-fold, P<0.01) and superoxide dismutase 2 (Sod2; FIG. 6D; PBS: −2.2-fold; H: 1.1-fold, P<0.05; H/VEGF: 1.6-fold, P<0.05), were also greatly expressed in both hydrogels.

Statistical Analysis

All the data are presented as mean±standard deviation. Prism 8 was used for statistical analyses by one- or two-way ANOVA, followed by Tukey's post-test (significant difference at P<0.05). Student's t-test was applied where appropriate.

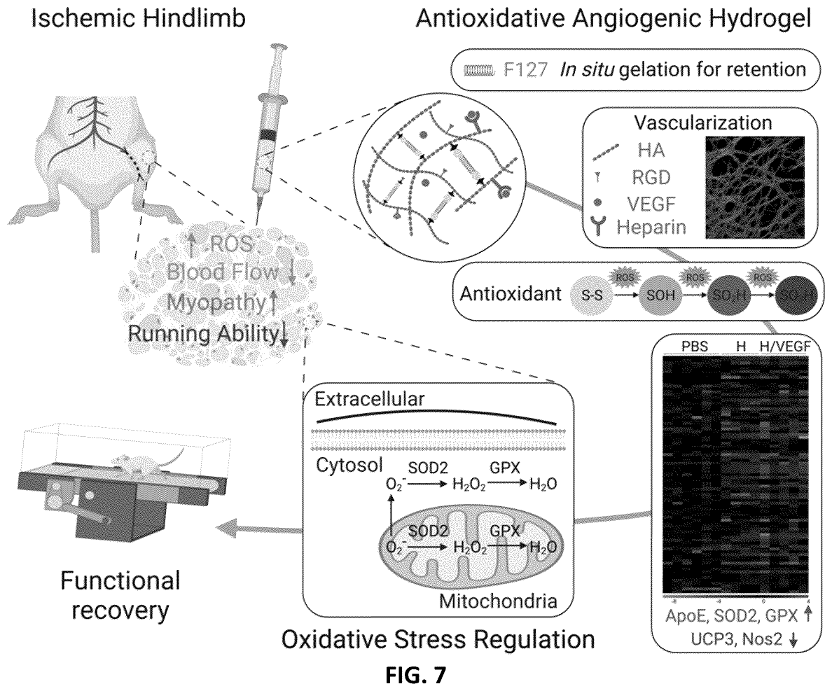

The results of these experiments confirmed the development of a distinctive HA hydrogel system to necessitate optimal delivery, retention, and therapeutic activity in the dynamic intramuscular environment of the PAD rat hindlimb. F127-SS-DA was used to cross-link HA-SH to form hydrogels with rapid gelation, minimal swelling, and skeletal muscle-matching stiffness. The F127-SS-DA cross-linked HA hydrogel has critical antioxidative effects both in vitro and in vivo. The HA hydrogels were further optimized with RGD to promote the formation of vascular-like structures and it was demonstrated that the optimized hydrogel preserved the vascular-like structures against H2O2-induced damage with reduced intracellular ROS levels. When injected into the ischemic muscles of the rat model of PAD, the HA hydrogel with the VEGF was able to regulate oxidative stress-related gene expression, increase local blood flow inside the muscle, and improve the running ability of the treated animals (FIG. 7).

### Example 2

To demonstrate the efficacy of RGD-modified PEEK devices at the anastomosis and endothelial cell adhesion, the following experiments were conducted.

RGD Supported Endothelial Cells to Form Vascular-Like Networks

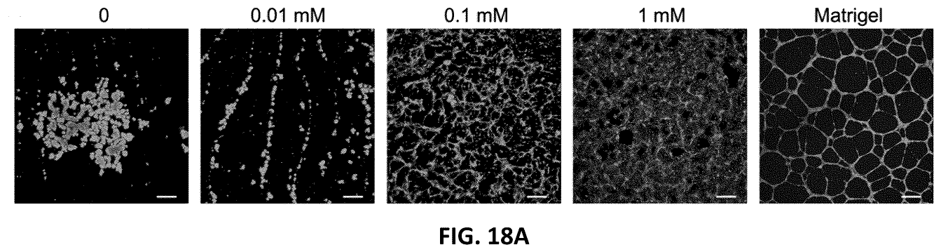

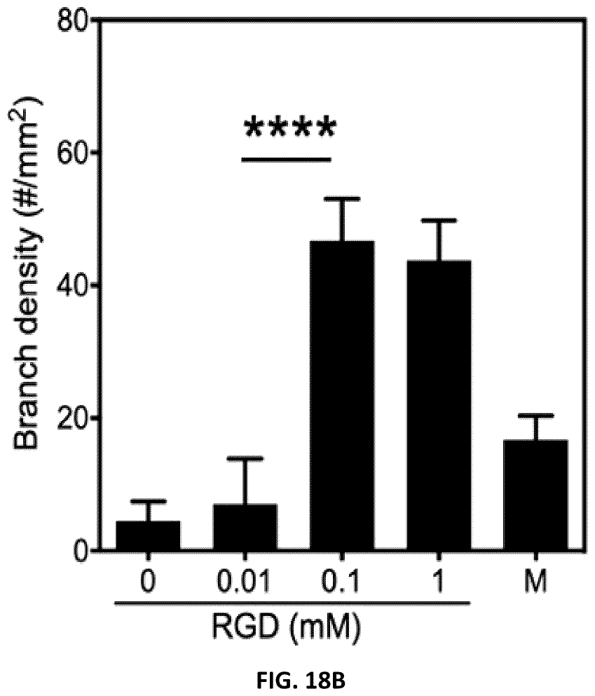

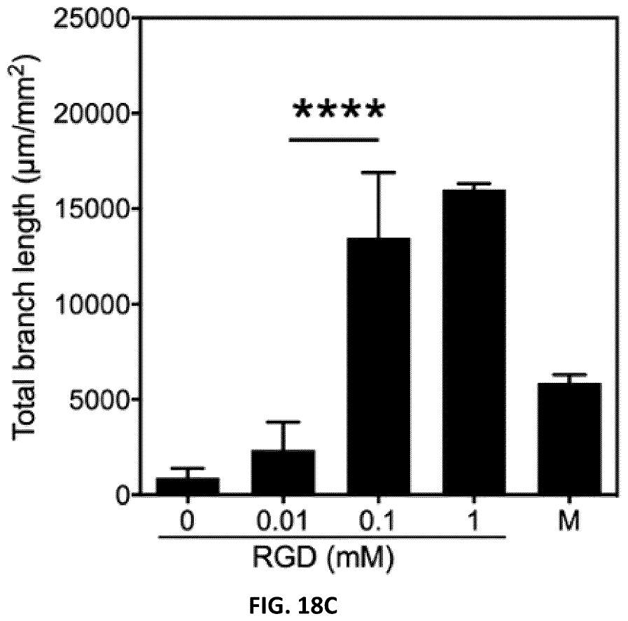

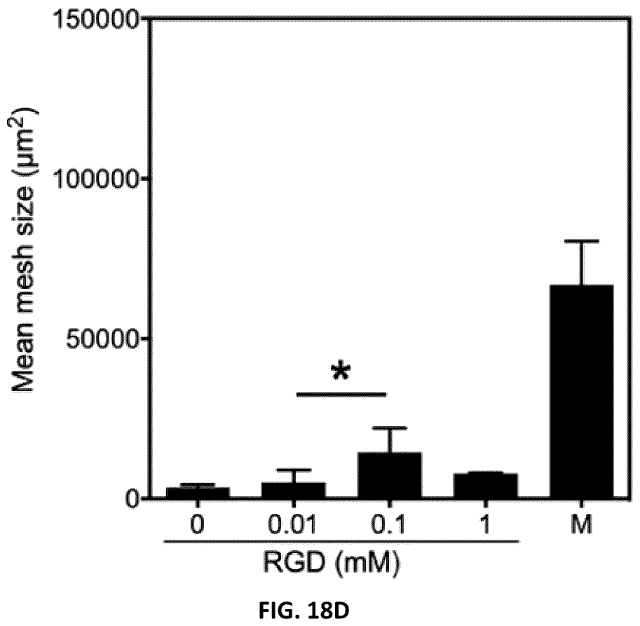

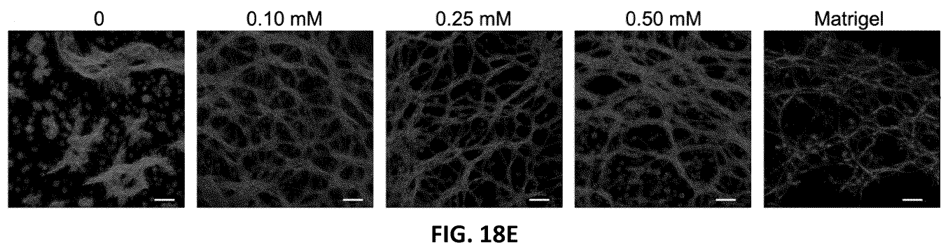

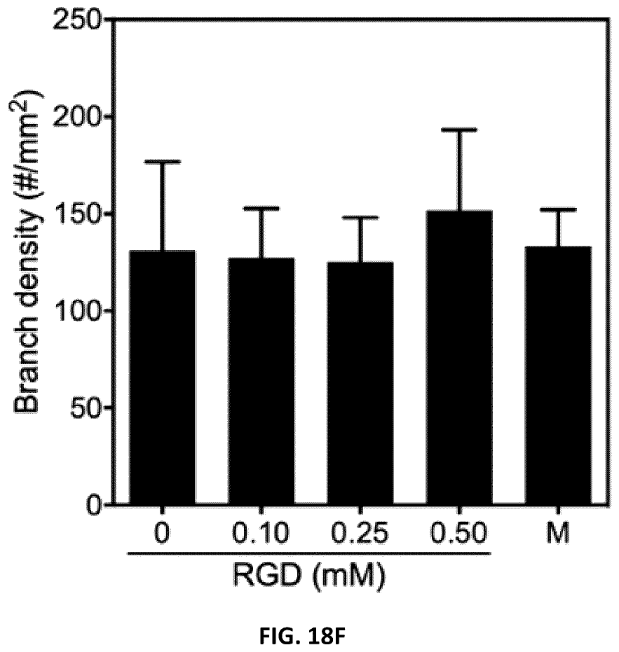

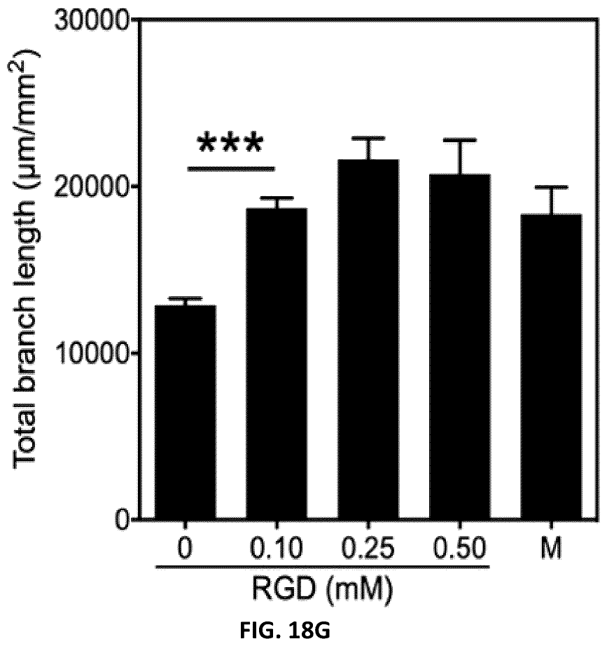

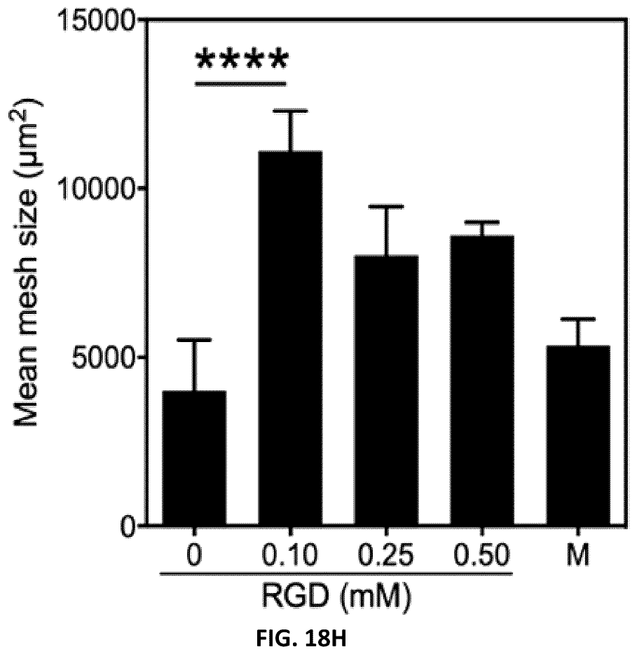

This Example demonstrates extensive experience in peptide synthesis and application for cell modulation. Specifically, RGD peptides have been synthesized by the solid phase method. It is demonstrated that RGD peptides, when conjugated to a hyaluronic acid-based hydrogel, improved endothelial cell adhesion in a dose-dependent manner (FIG. 18). RGD at 1 mM supported the formation of a full layer of endothelial cells on the hydrogel surface.

Surface Modification of PEEKs with Carboxylic Acid (COOH) Groups

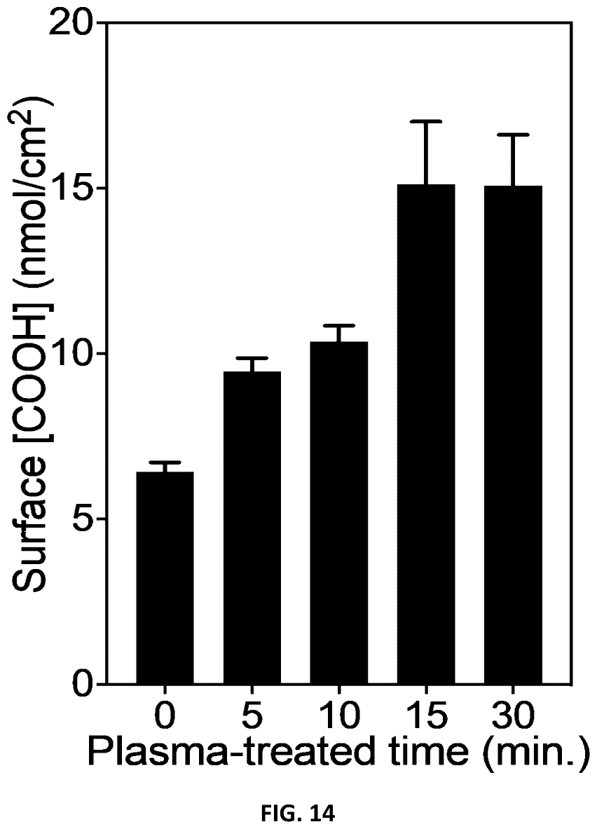

Plasma treatment was applied to modify polymer surfaces with specific chemical groups, such as amine or carboxylic acids (FIG. 14). Through these chemical groups, peptides, growth factors, or polysaccharides can be further conjugated to these modified polymers.

Surface Modification of PEEKs to Support Endothelial Cell Attachment

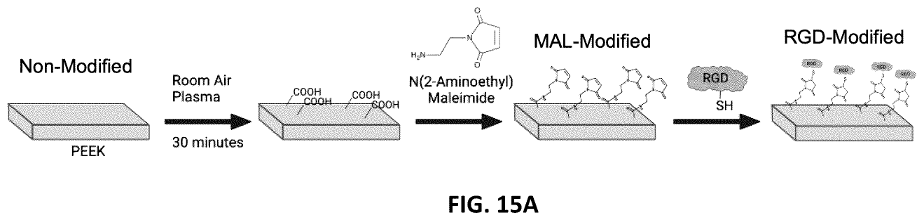

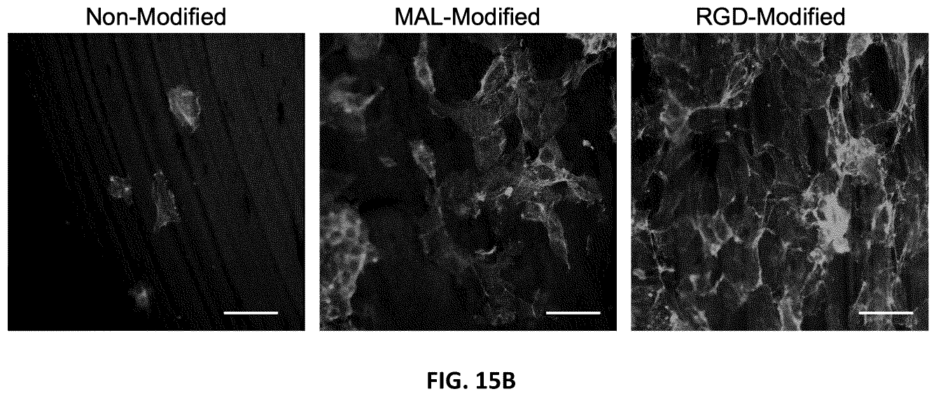

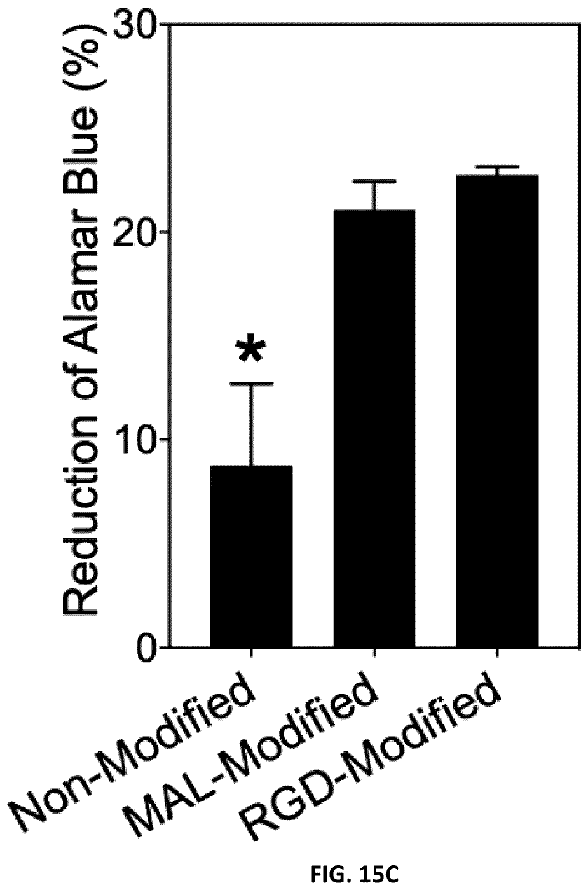

RGD peptides were chemically conjugated to PEEKs (FIG. 15). In specific, these carboxyl groups were converted into maleimide (MAL) by N-(2-aminoethyl)maleimide. Through these MAL groups, RGD could be further conjugated to these modified polymers. Human umbilical vein endothelial cells were cultured in three groups: Non-Treated, MAL-Treated, and RGD-Treated PEEK. The number of cells attached to these surfaces was quantitatively evaluated by the AlamarBlue assay. In terms of cell morphology, cells were fixed 6 hours post-incubation, stained with DAPI for nuclei and Alexa 488-Phalloidin for actin, and imaged with confocal microscopy. Surface modification experiments resulted in increased endothelial cell attachment to RGD- and Maleimide-Modified PEEK compared with Non-Modified PEEK (FIG. 15).

Vaso-Lock for Arterio-Venous Anastomosis

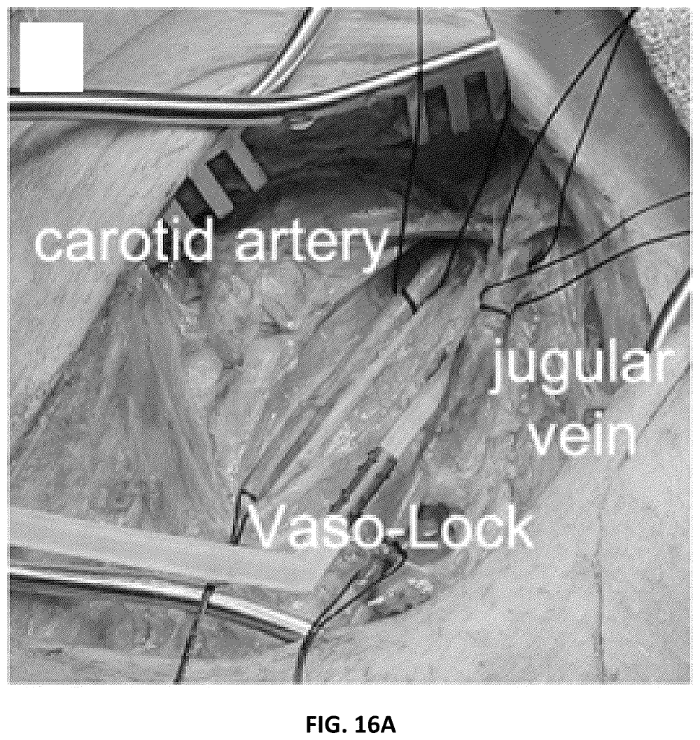

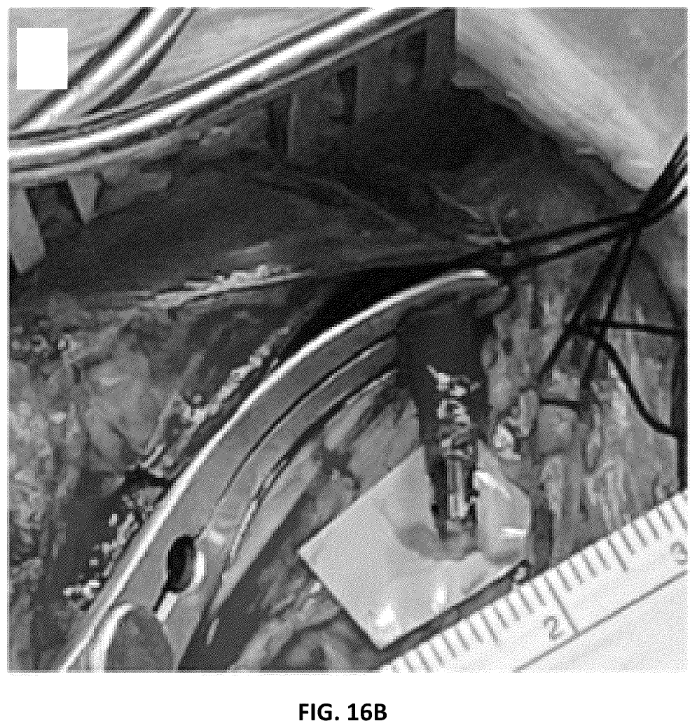

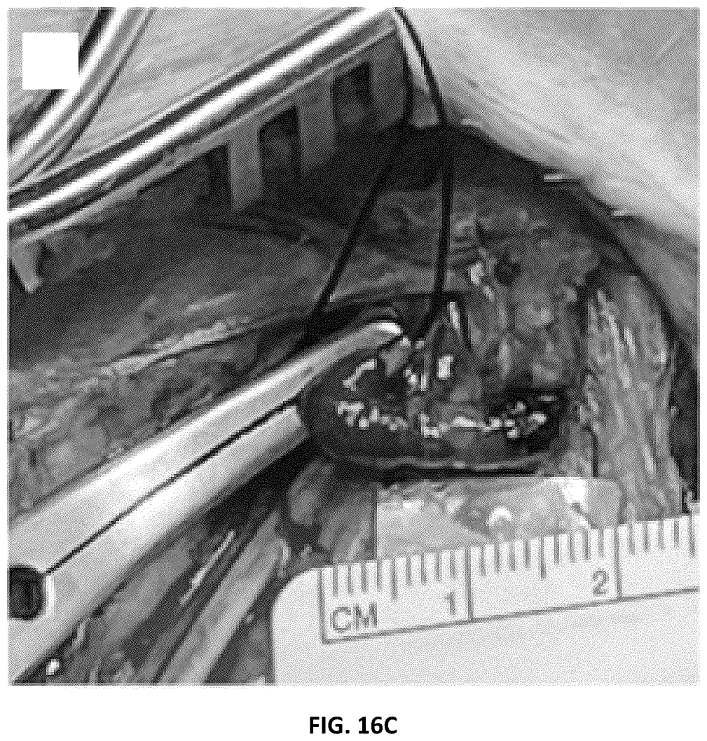

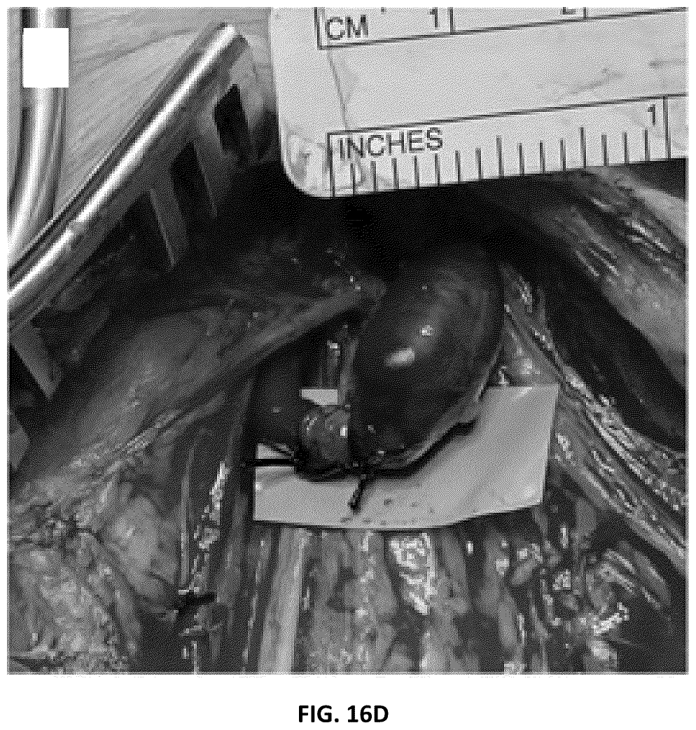

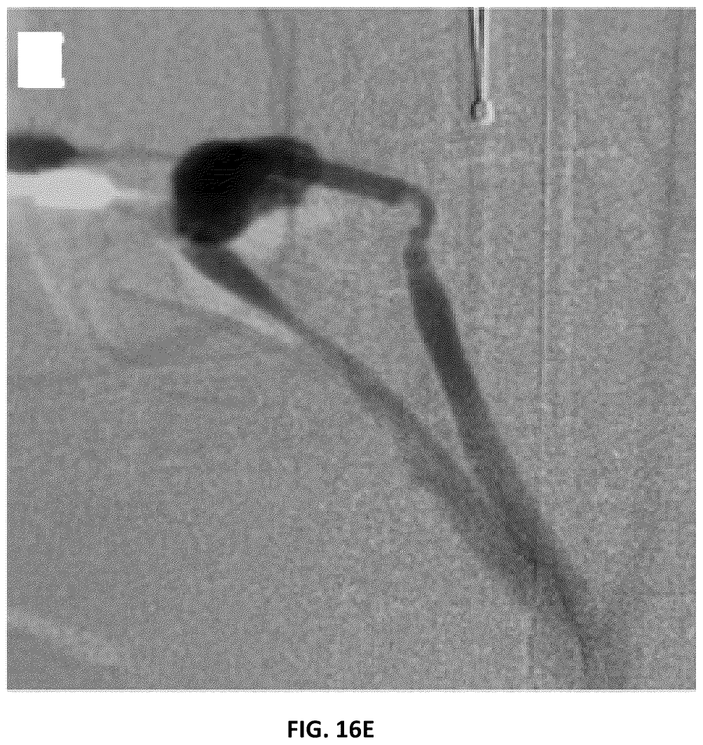

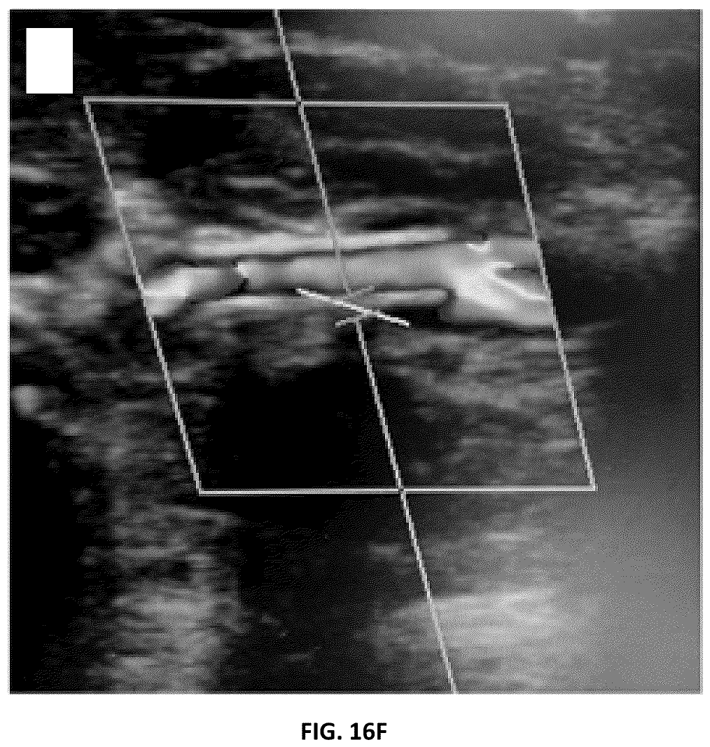

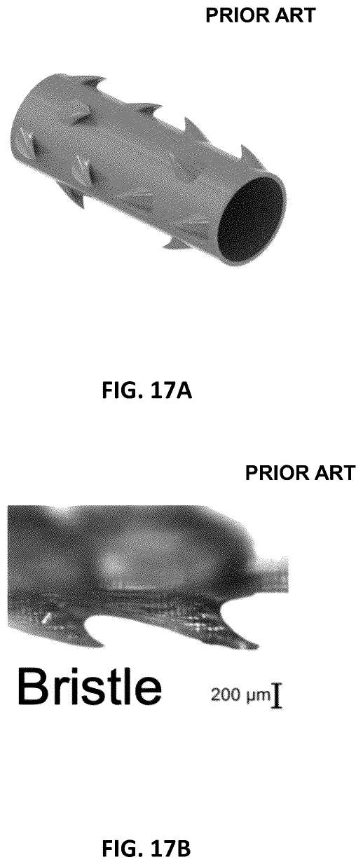

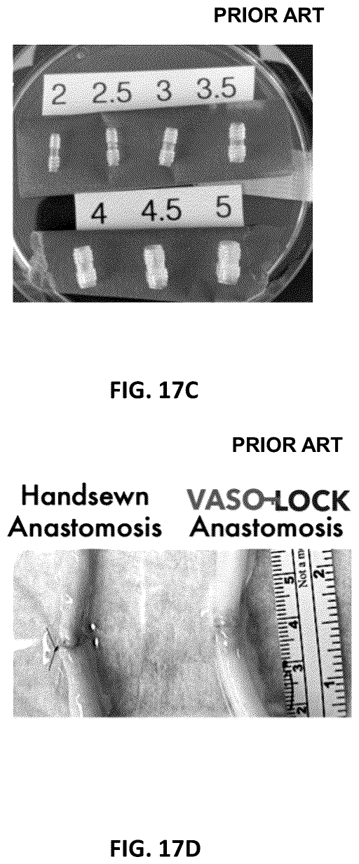

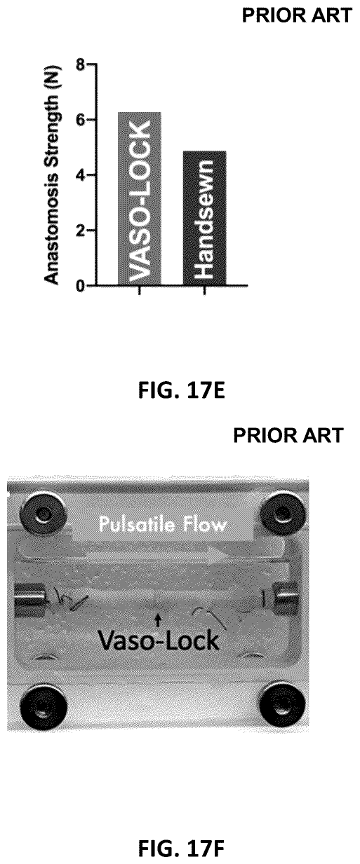

It was demonstrated that Vaso-Lock maintained anastomosis of the swine internal carotid artery and internal jugular vein (FIG. 16), with no evidence of leakage on angiogram after device deployment (FIG. 16E). Doppler ultrasonography showed the mixing of arterial and venous blood through the Vaso-Lock (FIG. 16F). The low blood flow rate inside the small-diameter vessels and turbulent flow at the sites of anastomosis may lead to a high risk of luminal thrombosis. It is proposed to place Vaso-Locks in situations where they might be challenged, and this arteriovenous loop model would provide a high-stress environment to test the effectiveness of the surface modification in preventing thrombosis and encouraging endothelialization.

### Example 3—Anastomosis Device Surface-Modified with Cyclic

Peptides

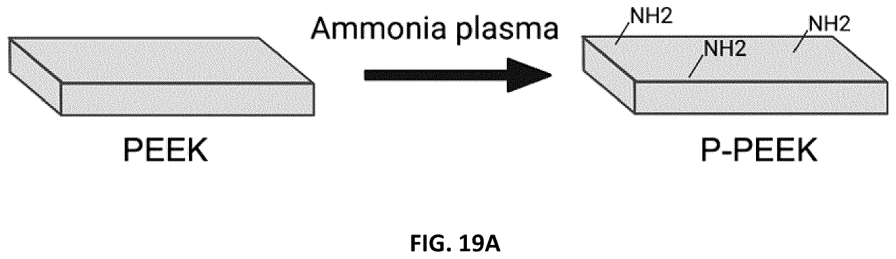

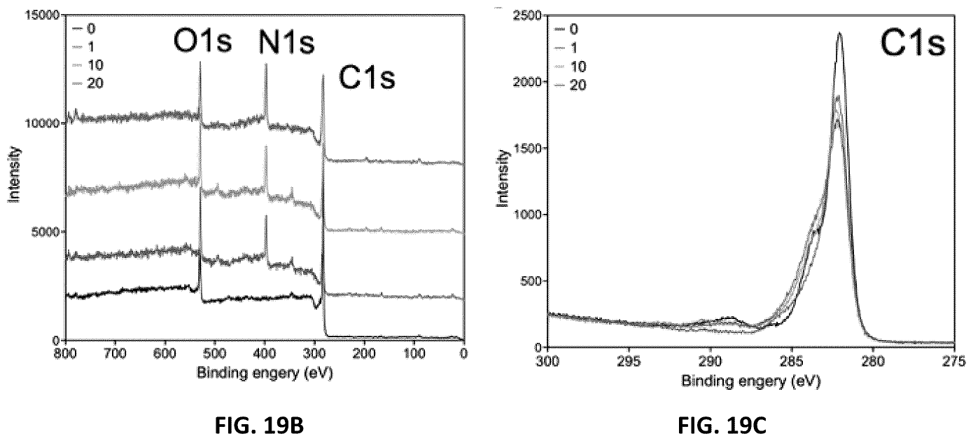

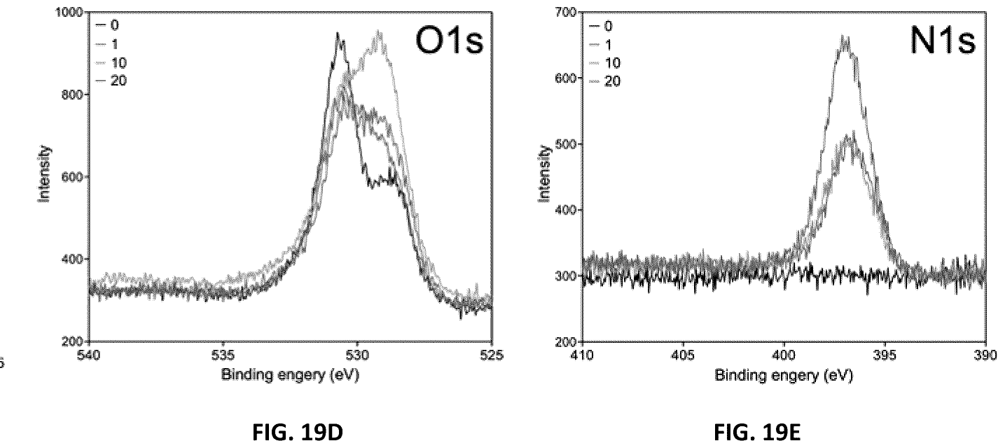

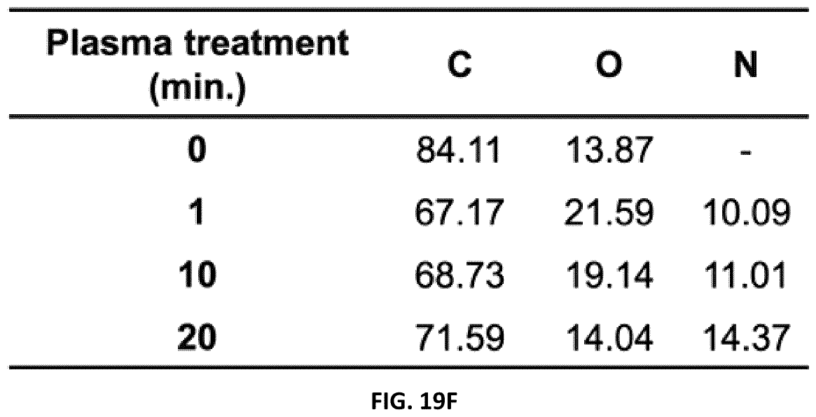

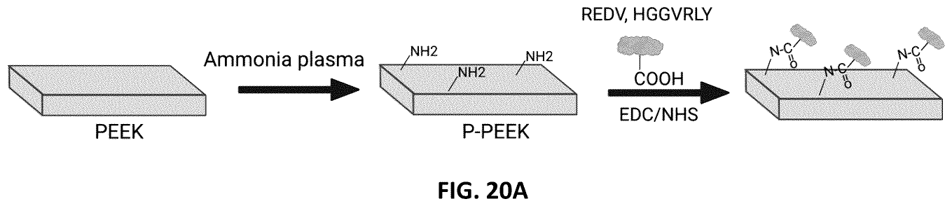

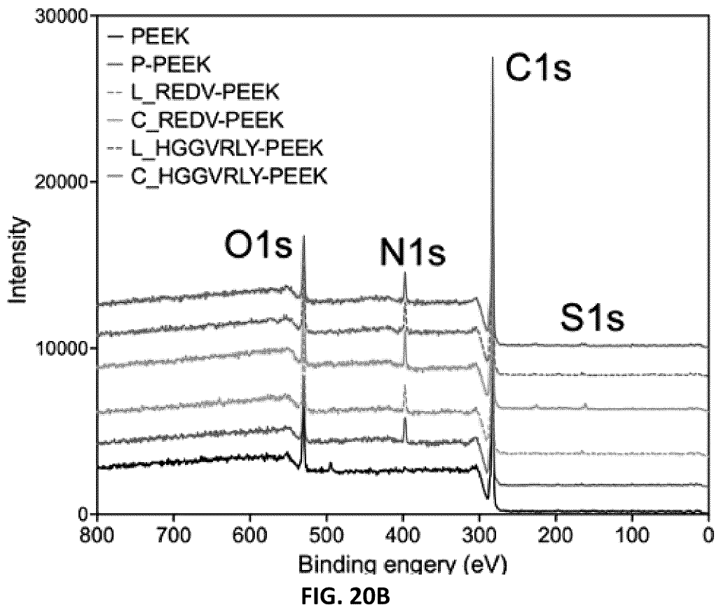

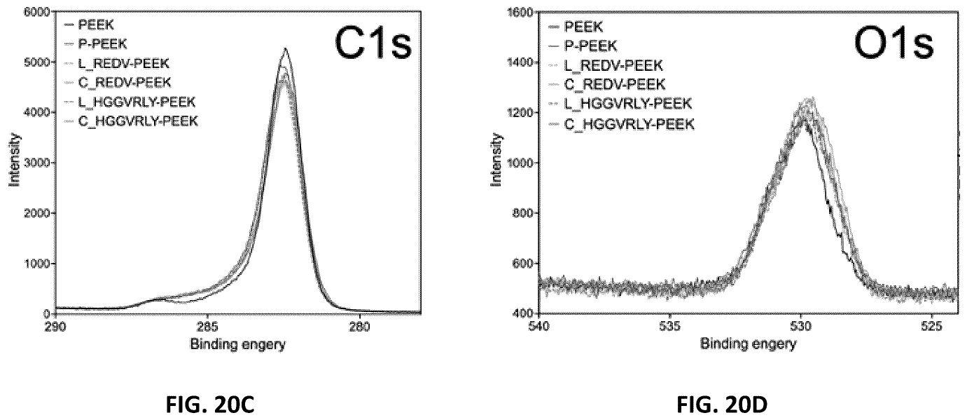

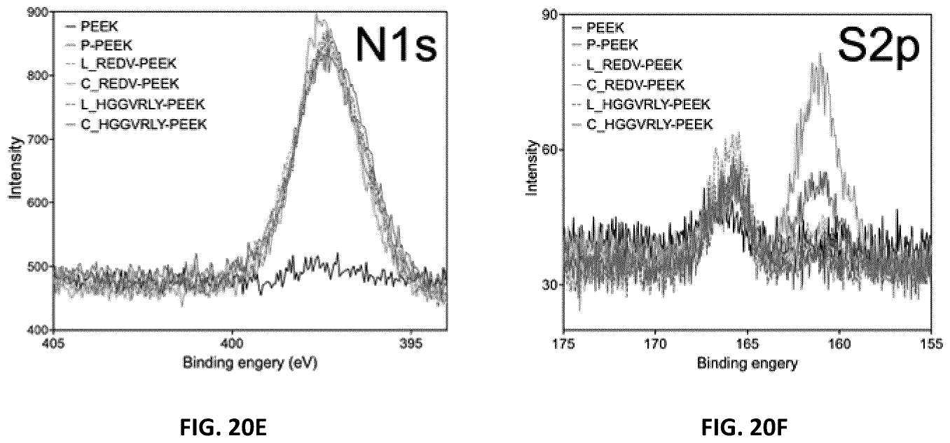

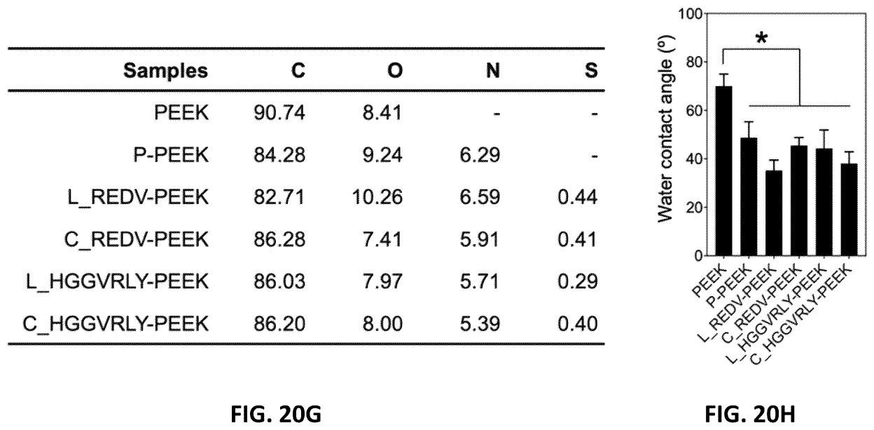

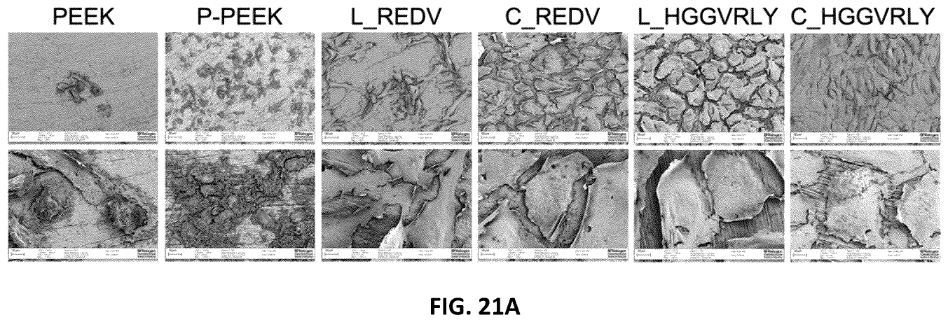

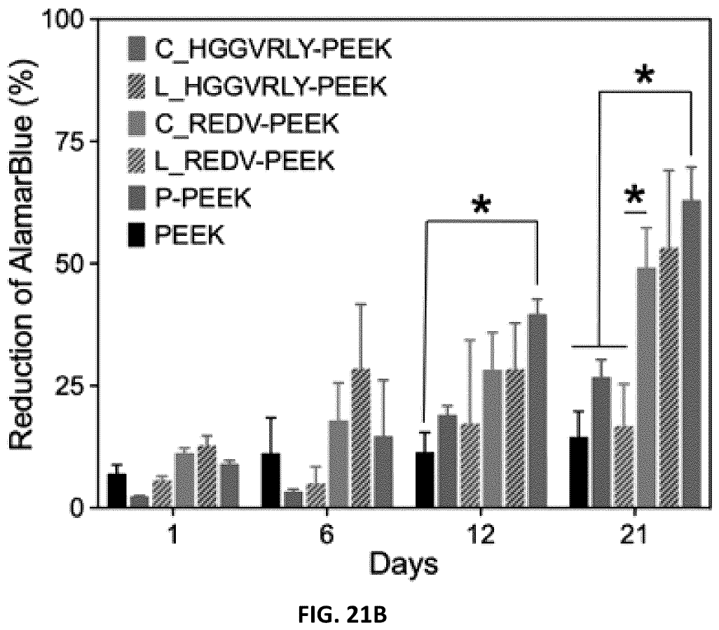

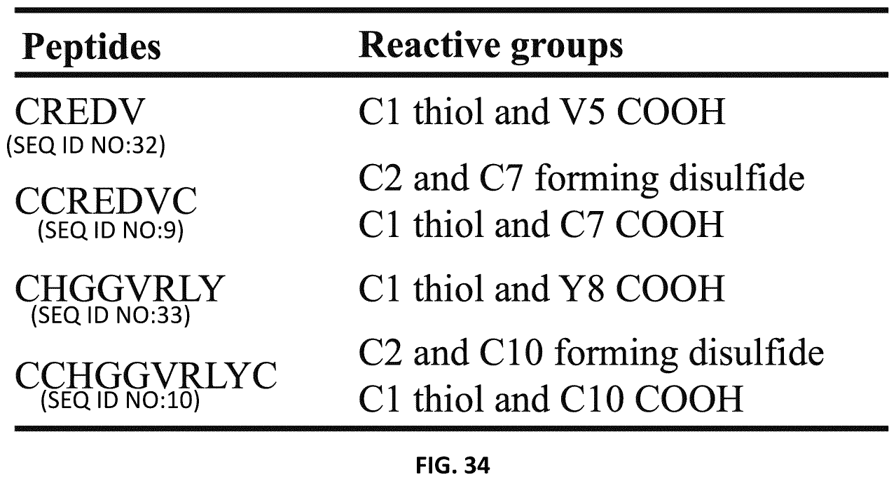

In this example, surfaces of an anastomosis device are modified with cyclic peptides (FIG. 34, SEQ ID NO:32, SEQ ID NO:9, SEQ ID NO:33, SEQ ID NO:10), and their functionality is characterized. Polyether ether-ketone (PEEK) surfaces are plasma treated to create reactive amines (FIG. 19A), which was confirmed by XPS (FIG. 19B-F). Peptides are then reacted onto the surface through reactive COOH group with EDC/NHS chemistry (FIG. 20A), which was confirmed by XPS (FIG. 20B-H). These surface-modified PEEKs supported endothelial cell attachment and proliferation. More specifically, a significantly larger number of HUVECs showed on the cyclic and linear HGGVRLY-modified PEEKs compared to the non-modified PEEK on Day 21. Cyclic REDV-modified PEEK also supported a larger number of HUVECs when compared to the linear REDV-modified PEEK or the non-modified PEEK (FIG. 21A-B).

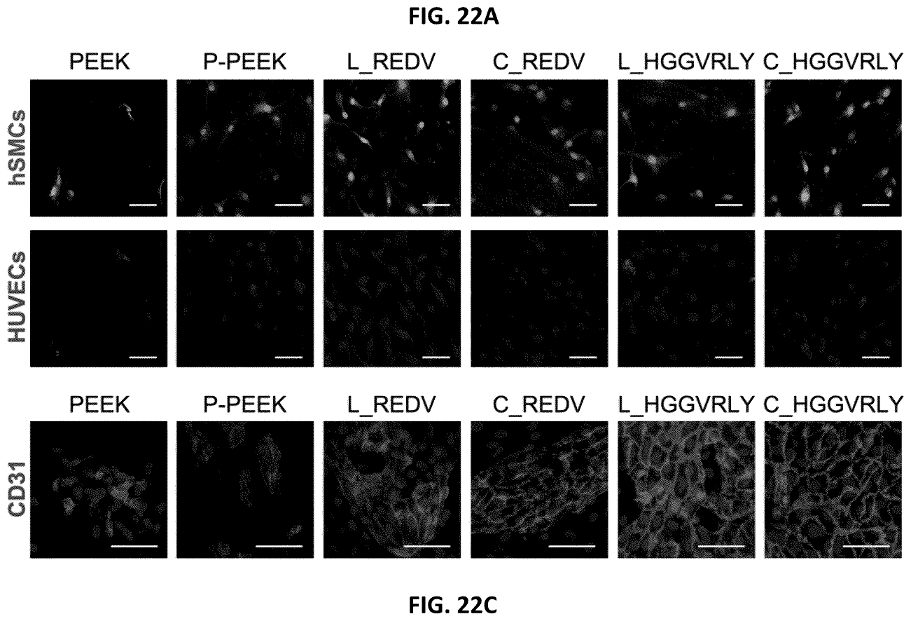

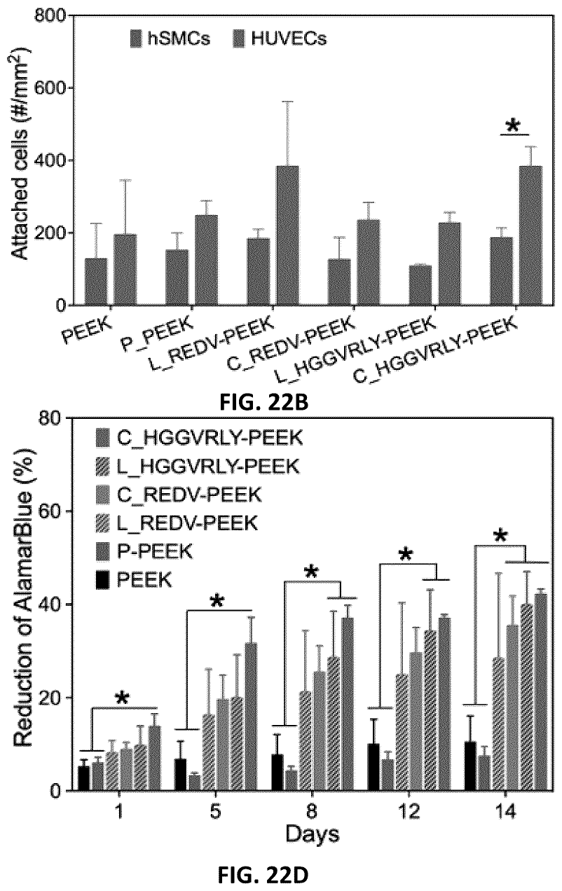

Further, surface-modified PEEKs supported endothelium formation. First, when HUVECs and hSMCs were co-cultured (FIG. 22A), a larger number of HUVECs than hSMCs appeared on the surface of cyclic HGGVRLY-modified PEEK on Day 1 (FIG. 22B). Endothelium formation on the surface of PEEKs was imaged at Day 14 (FIG. 22C), and cell proliferation on PEEKs was quantified (FIG. 22D). At Days 1 and 5, a large number of cells appeared on the cyclic HGGVRLY-modified PEEK compared to the plasma- and non-modified PEEKs.

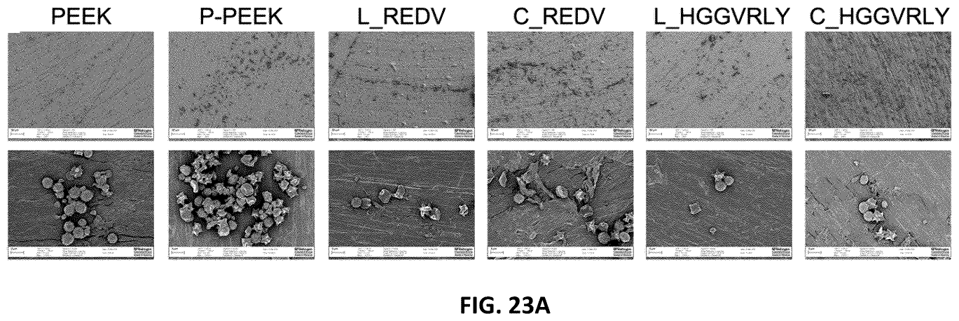

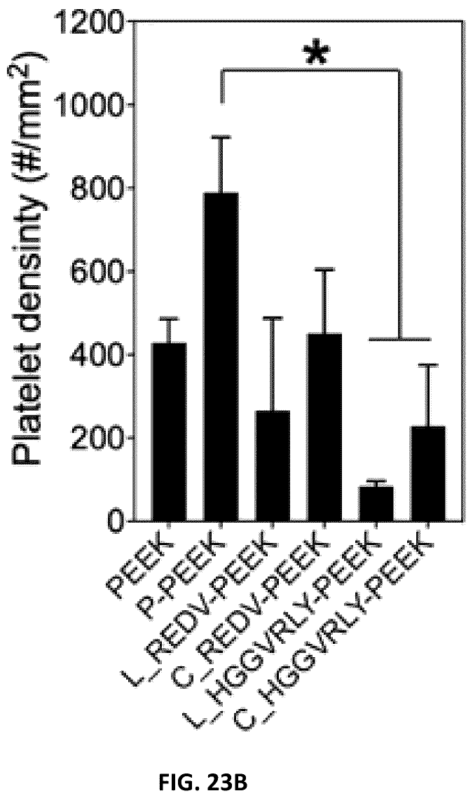

At Days 8 and 12, the linear and cyclic HGGVRLY-modified PEEK showed a larger number of cells compared to the plasma- and non-modified PEEKs. At Day 15, cyclic REDV-, linear HGGVRLY-, and cyclic HGGVRLY-modified PEEKs showed a larger number of cells compared to the plasma- and non-modified PEEKs. PEEKs were then incubated with pig platelet-rich plasma and SEM was performed (FIG. 23A), where a larger number of platelets on the plasma-modified PEEK were observed when compared to the linear or cyclic HGGVRLY-modified PEEKs (FIG. 23B).

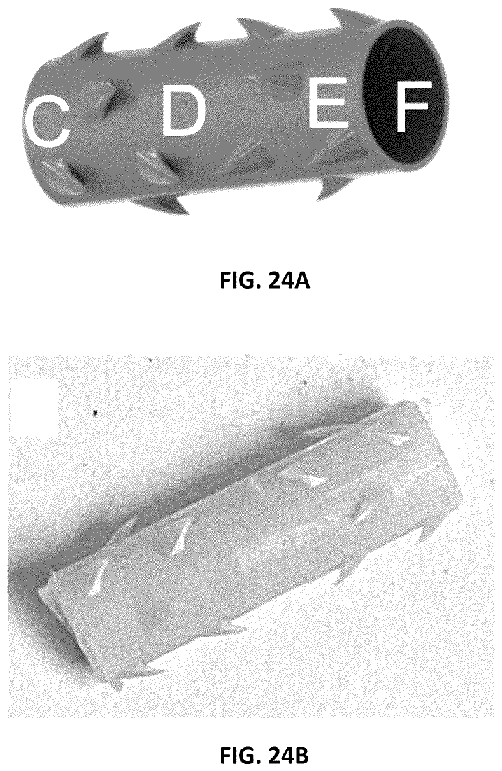

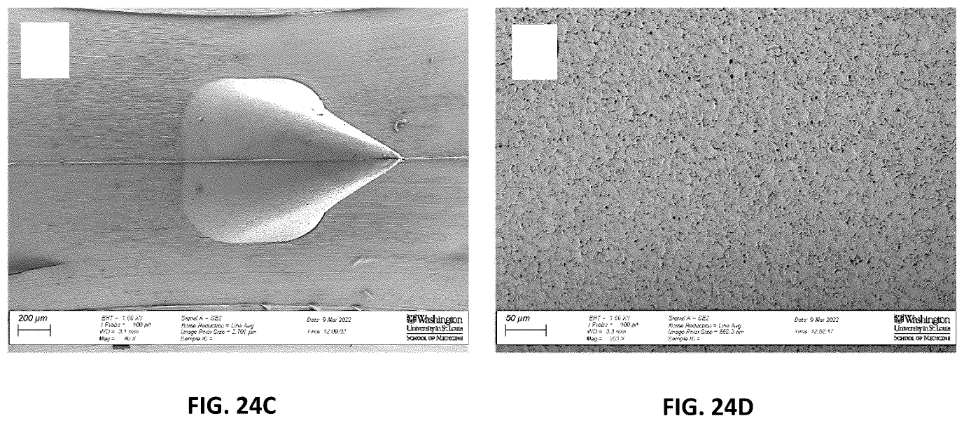

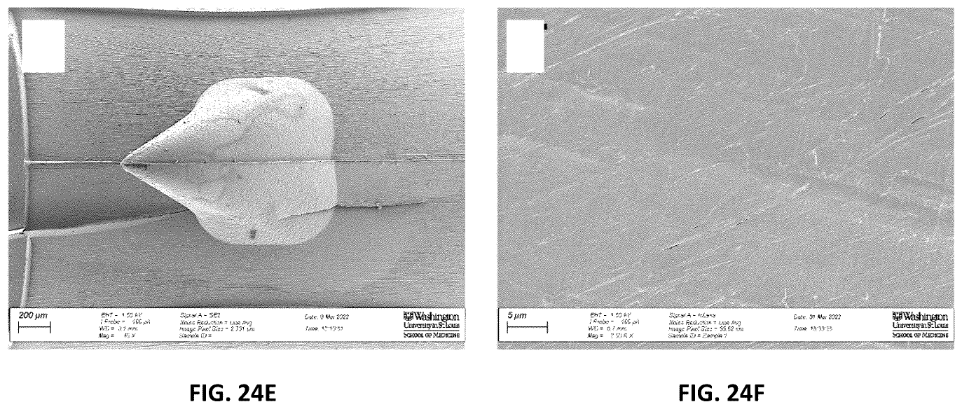

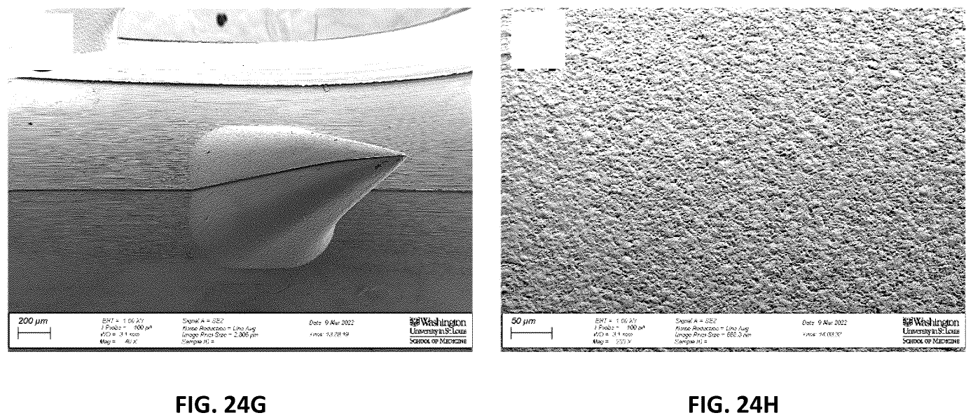

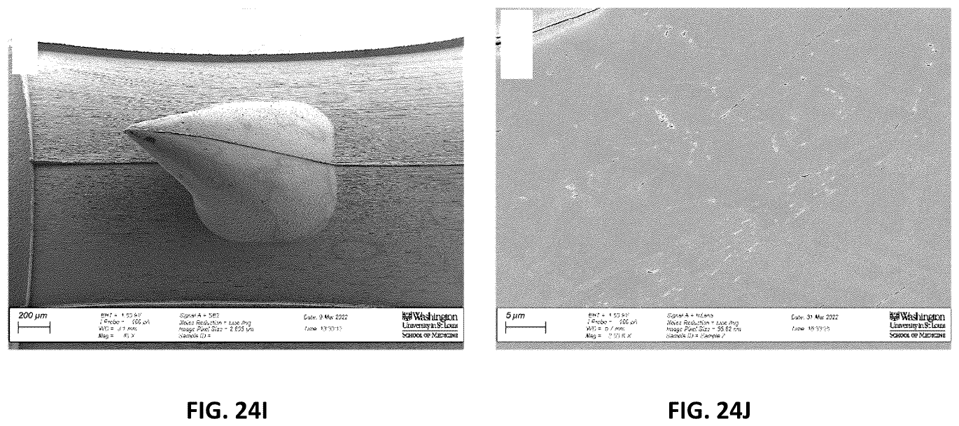

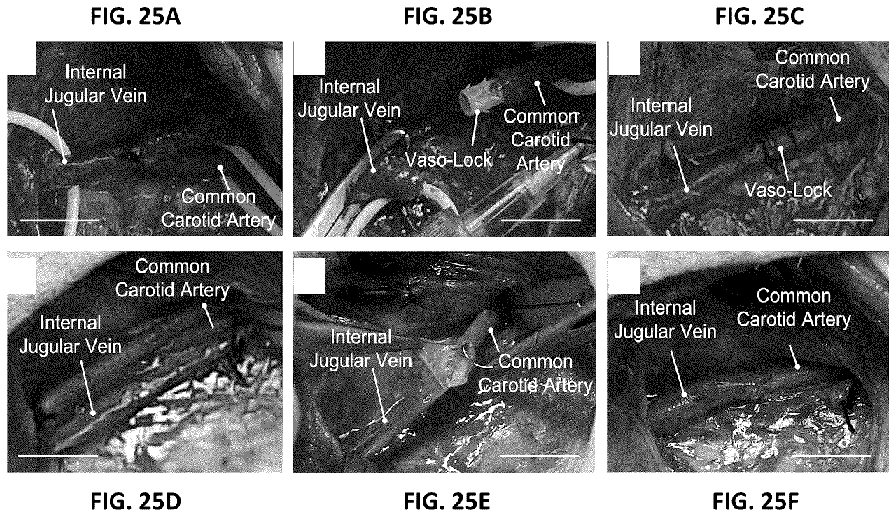

A Vaso-Lock anastomosis device was then designed (FIG. 24A) and fabricated with injection molding (FIG. 24B). Close-up images of the anchors (FIGS. 24C, G, E, and I), the outer surface (FIGS. 24D and H), and inner surface (FIGS. 24F and J) before (FIG. 24C-F) and after (FIG. 24G-J) ethylene oxide sterilization can be seen in FIG. 24. The Vaso-Lock was then used in an anastomosis procedure of the swine common carotid artery and internal jugular vein (FIG. 25A-C). First, the common carotid artery and internal jugular vein were identified for anastomosis for Vaso-Lock (FIG. 25A). Then the Vaso-Lock was placed in the common carotid artery, and then the arterio-venous anastomosis was performed with Vaso-Lock. As an alternative, a hand-sewn anastomosis was also performed (FIG. 25D-F). First, the common carotid artery and internal jugular vein were identified for hand-sewn anastomosis (FIG. 25D). Then, sutures are applied for anastomosis (FIG. 25E), and arterio-venous anastomosis by sutures was performed (FIG. 25F).

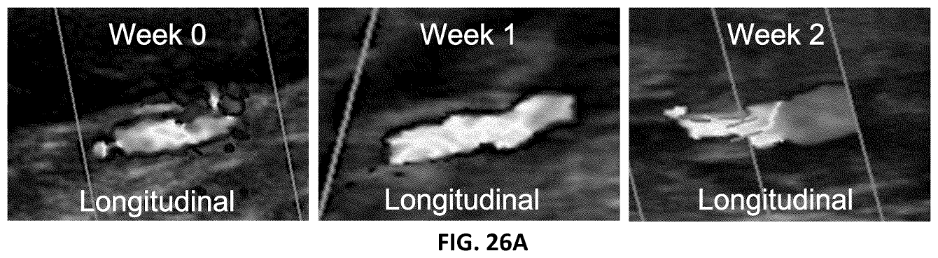

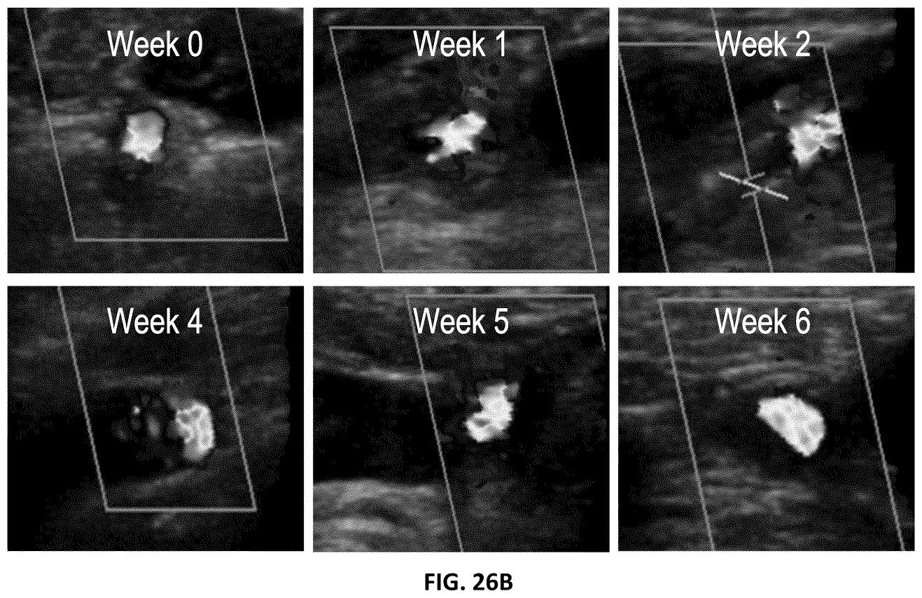

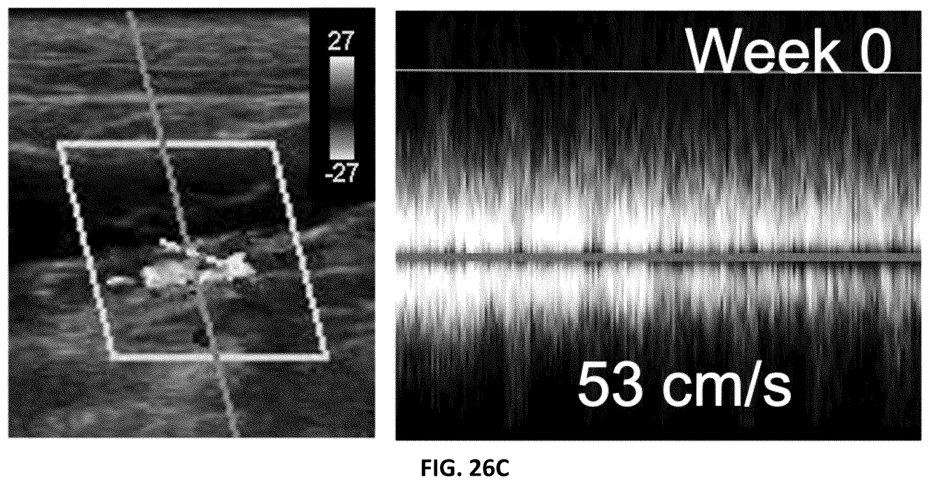

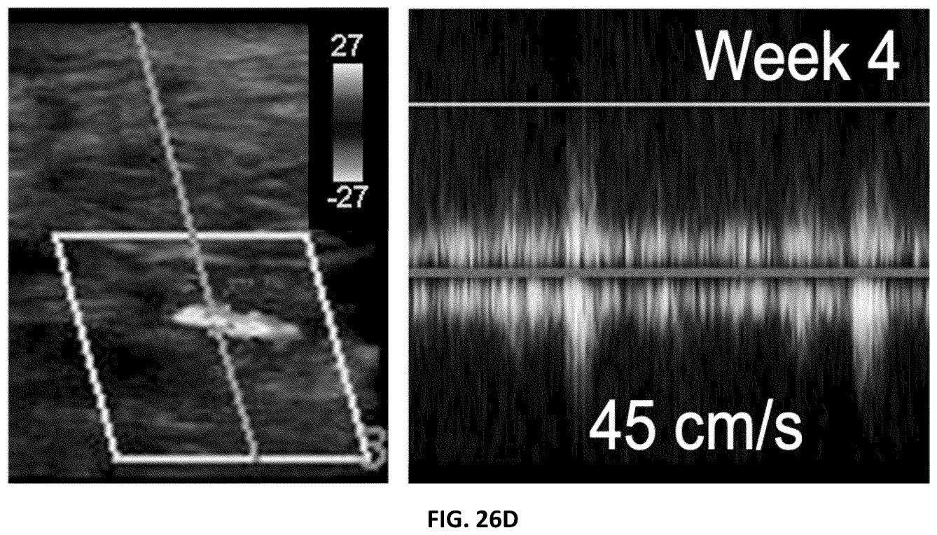

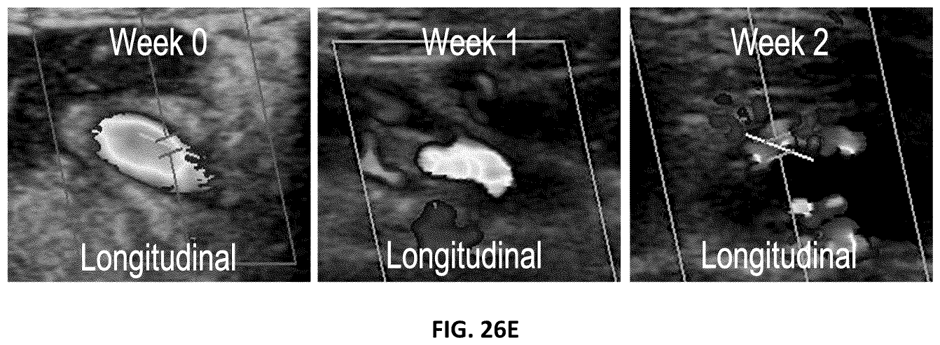

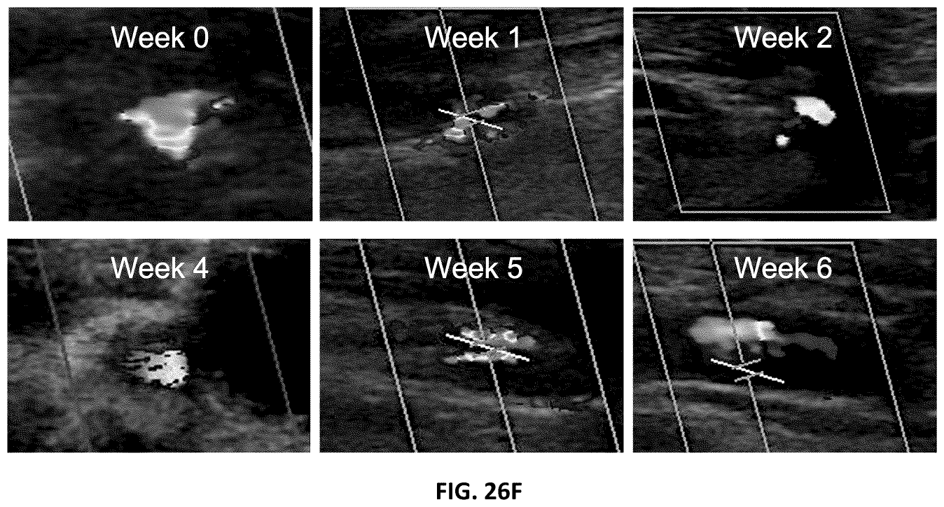

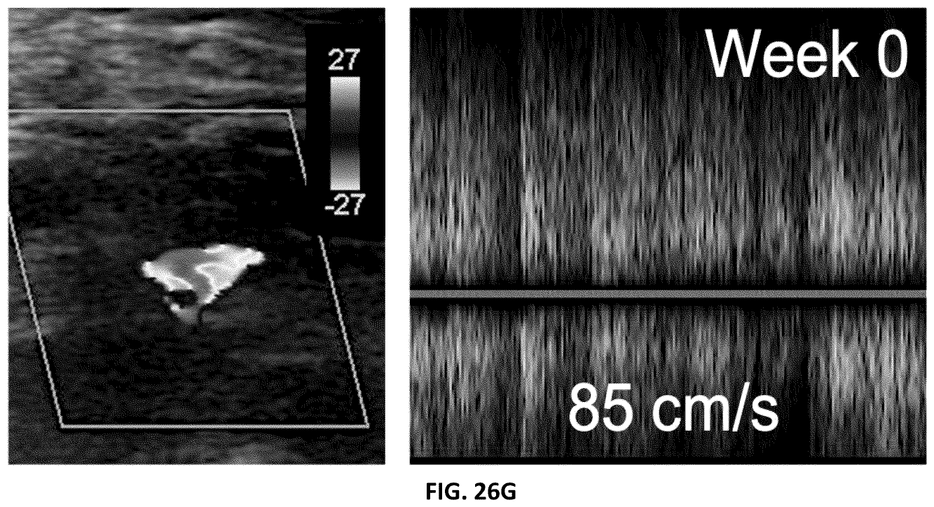

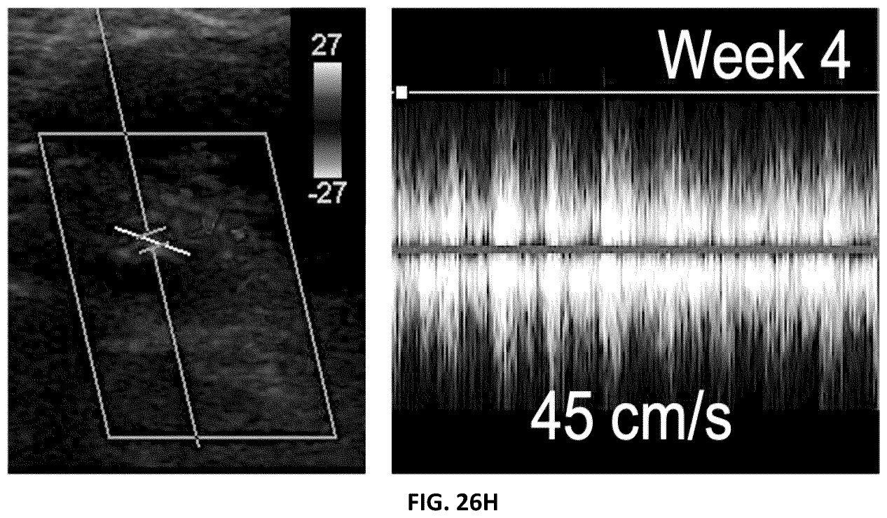

Doppler ultrasonography was then performed on both Vaso-Lock (FIG. 26A-D) and hand-sewn (FIG. 26E-H) anastomosis sites. Vaso-Lock sites were imaged over 2 weeks (FIG. 26A) and 6 weeks (FIG. 26B). Then blood velocity was checked by pulse wave spectrum within the Vaso-Lock after implantation at Weeks 0 (FIG. 26C) and 4 (FIG. 26D). Similarly, hand-sewn anastomosis sites were imaged over 2 weeks (FIG. 26E) and 6 weeks (FIG. 26F), and blood velocity was checked by pulse wave spectrum within the Vaso-Lock after implantation at Weeks 0 (FIG. 26G) and 4 (FIG. 26H).

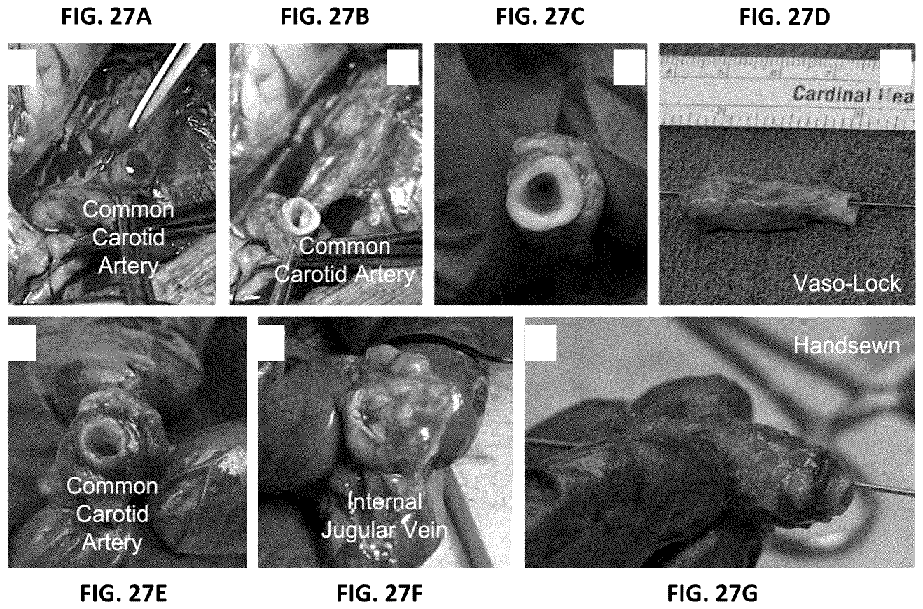

In situ patency of Vaso-Lock (FIG. 27A-D) and hand-sewn (FIG. 27E-G) anastomosis were then evaluated at 6 weeks. In the Vaso-Lock method, first, flowing blood was identified in the common carotid artery (FIG. 27A-B). Then, the vessel lumen was viewed at the common carotid artery side (FIG. 27C). Finally, the guidewire went through the explanted blood vessels anastomosed by the Vaso-Lock (FIG. 27D). In the hand-sewn method, first, the vessel lumen was viewed at the common carotid artery (FIG. 27E) and internal jugular vein side (FIG. 27F), respectively. Finally, the guidewire went through the explanted blood vessels anastomosed by sutures (FIG. 27G).

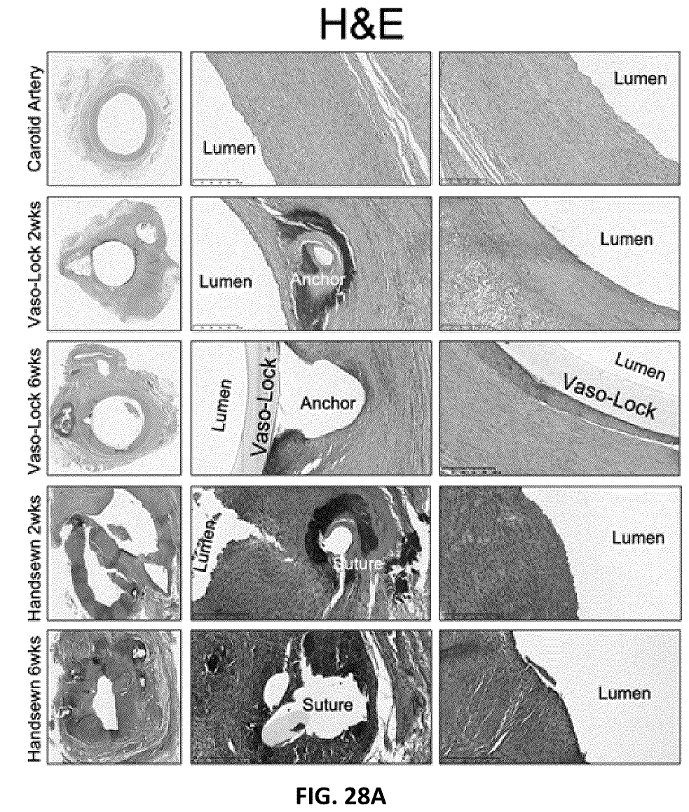

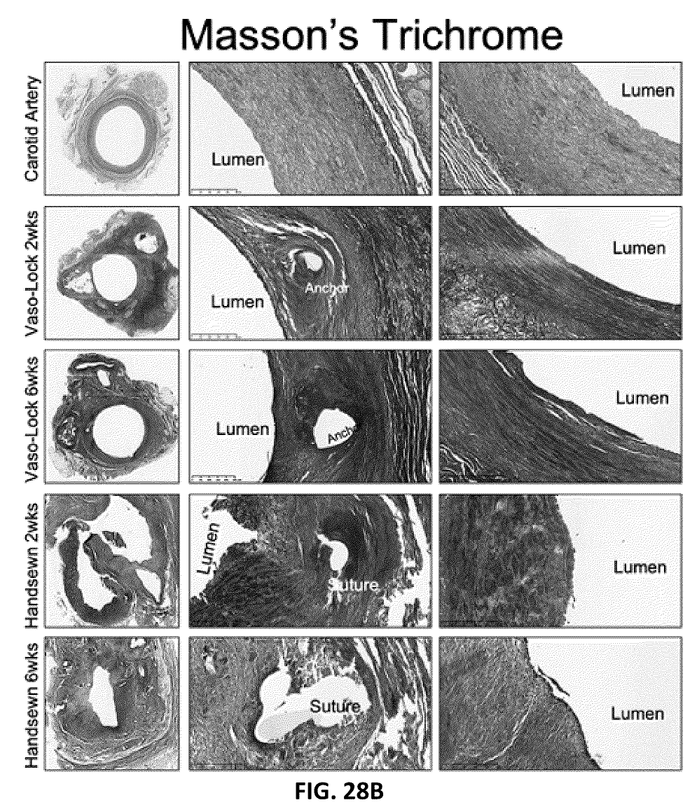

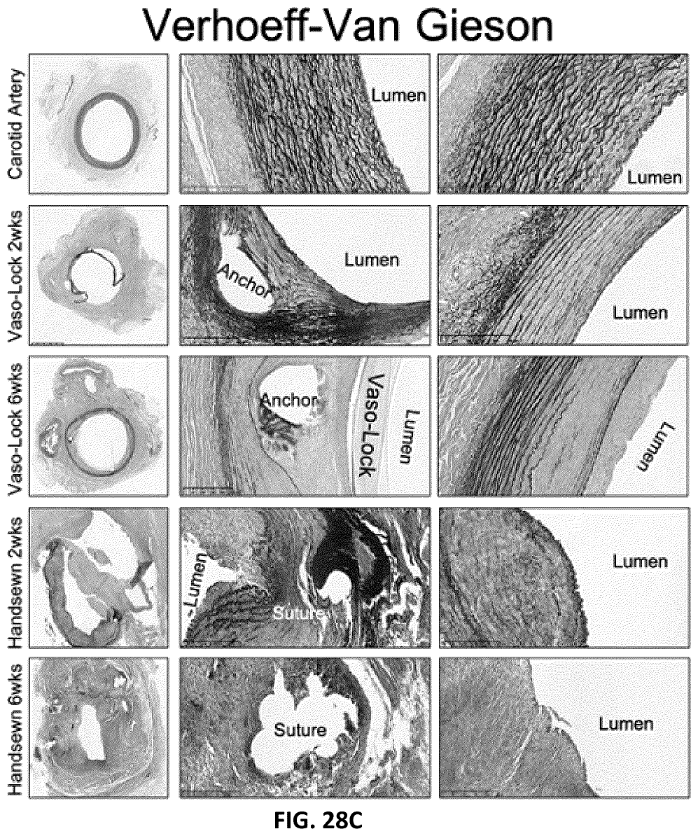

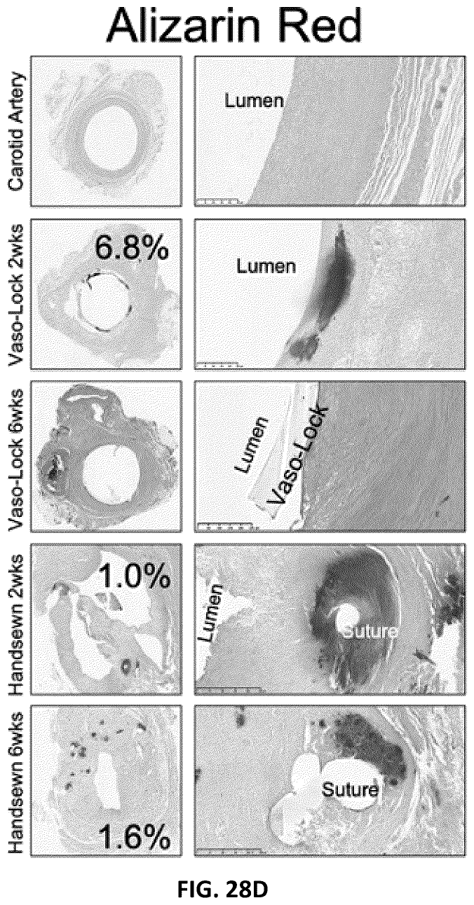

Histological analysis of the Vaso-Lock and hand-sewn was then performed on explanted blood vessels at Weeks 2 and 6. H&E staining (FIG. 28A), Masson's Trichrome staining (FIG. 28B), with collagen stained in blue, Verhoeff-Van Gieson staining (FIG. 28C), with elastin stained in black, and Alizarin Red staining (FIG. 28D) was performed on both Vaso-Lock and hand-sewn anastomosis sites at weeks 2 and 6. Calcium accumulation (dark red in FIG. 28D) was identified for the Vaso-Lock sample at Week 2, and hand-sewn samples at Weeks 2 and 6, respectively. Calcium was quantified and indicated in the FIG. 28D.

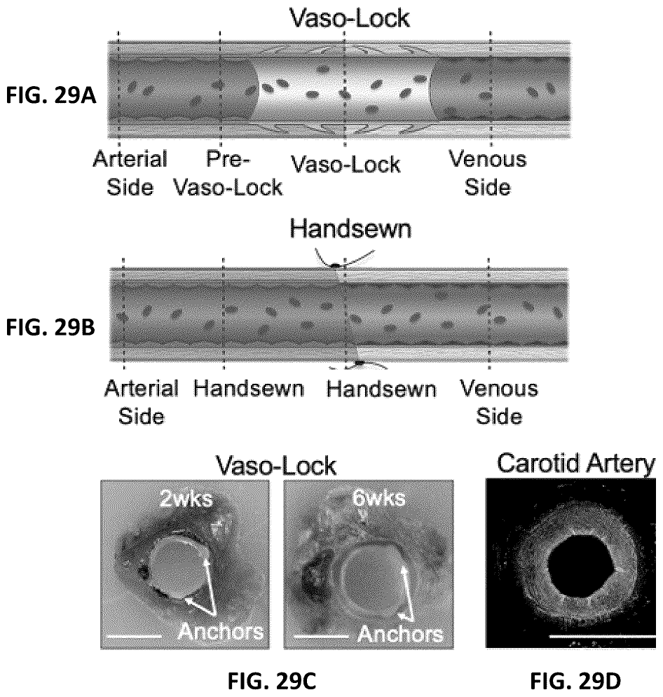

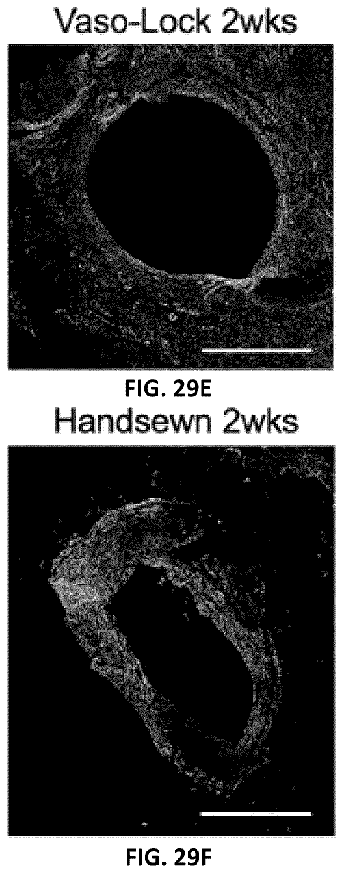

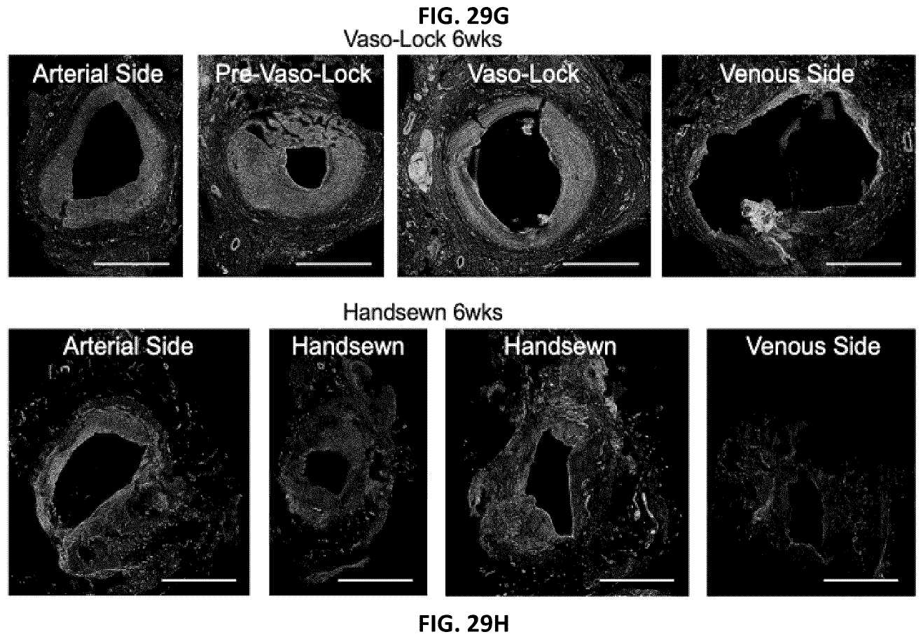

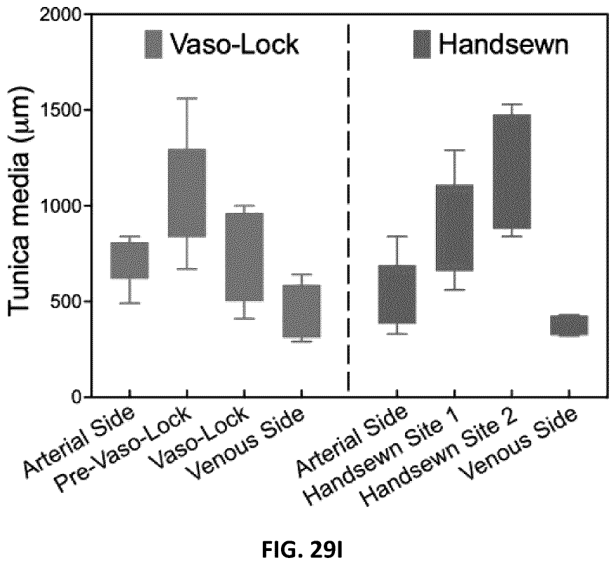

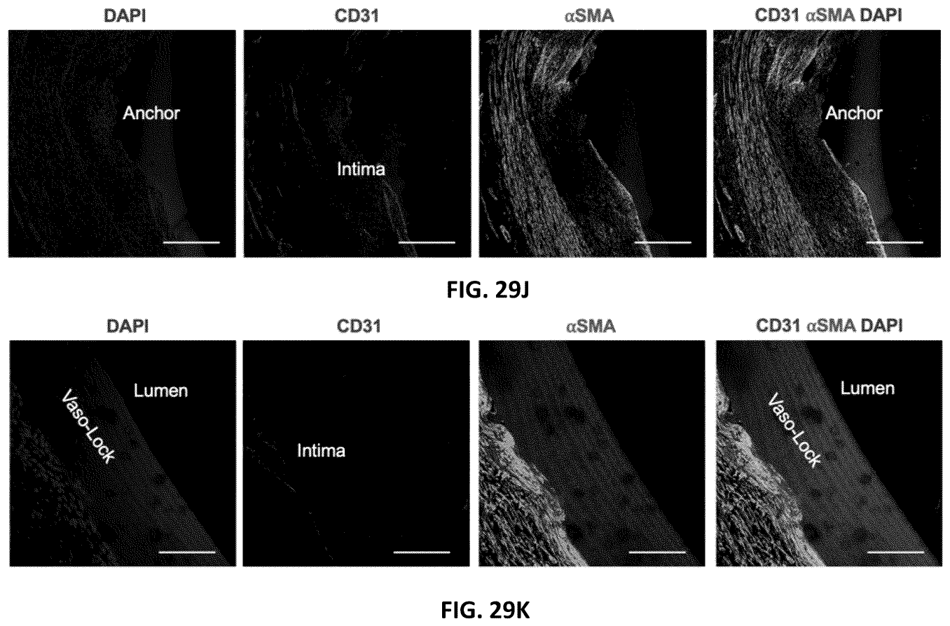

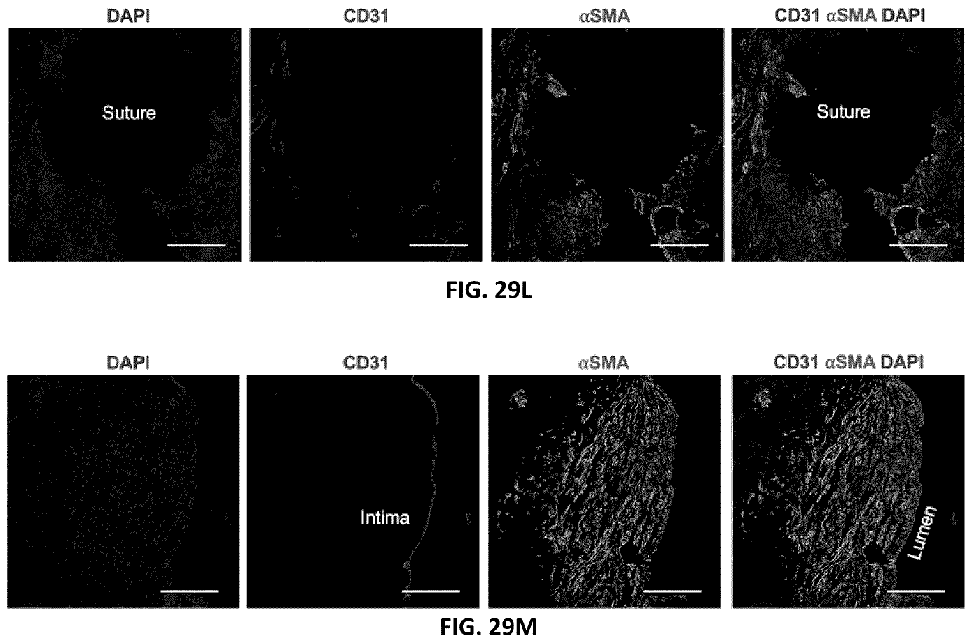

Immunohistology analysis of Vaso-Lock (FIG. 29A) and hand-sewn (FIG. 29B) anastomosed blood vessels were then performed at Weeks 2 and 6. Tissue blocks indicated that Vaso-Lock joined blood vessels by anchors (arrows) at Weeks 2 and 6 (FIG. 29C), which produced a normal common carotid artery with a uniform tunica media (FIG. 29D). Anastomosed blood vessels maintained an open lumen by both Vaso-Lock (FIG. 29E) and hand-sewn (FIG. 29F) at Week 2. A series of tissue sections of blood vessels anastomosed by Vaso-Lock (FIG. 29G) and hand-sewn (FIG. 29H) were imaged at Week 6. Tunica media of tissue sections was quantified at Week 6, and intimal growth was identified at the arterial side (FIG. 29I). The anchors of the Vaso-Lock penetrated the tunica intima into media (FIG. 29J-K), and sutures were identified in the middle layer of the common carotid artery (FIG. L-M).

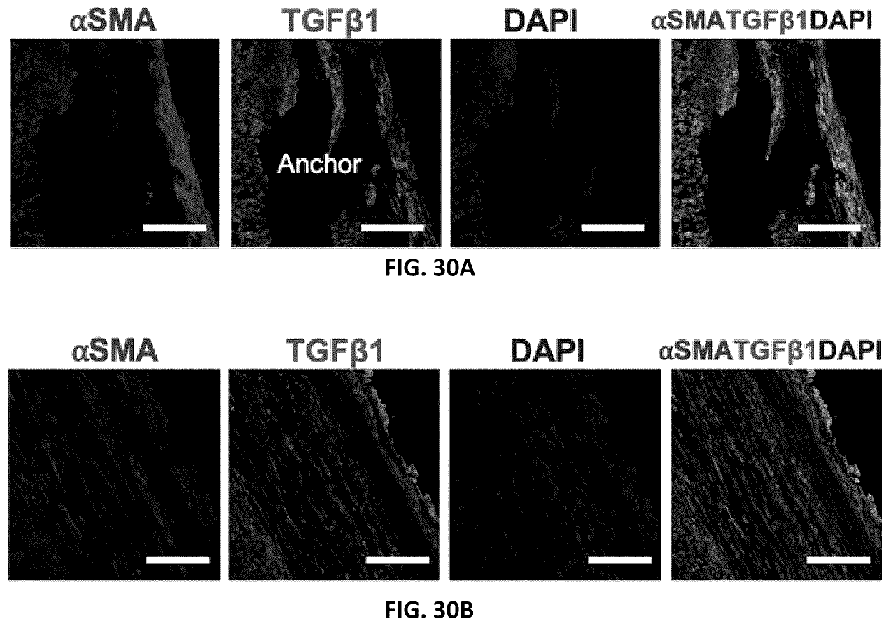

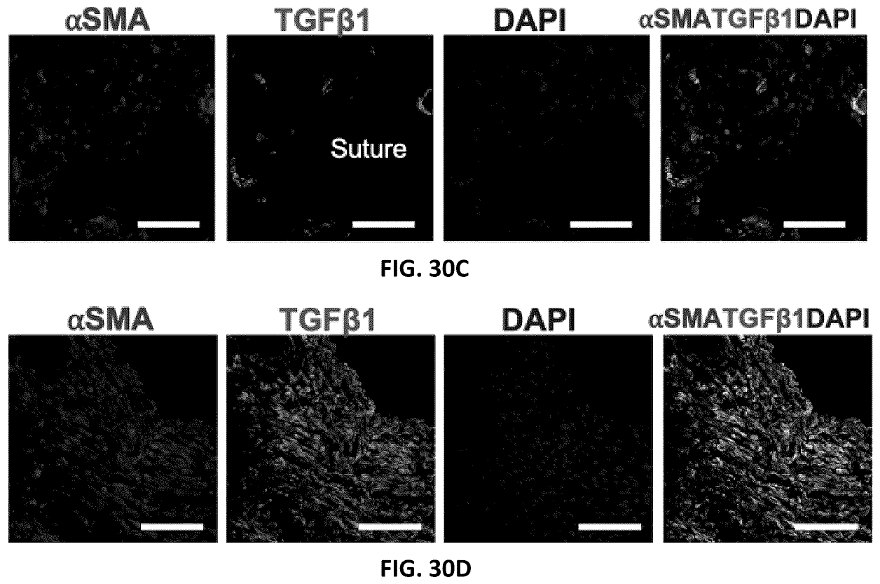

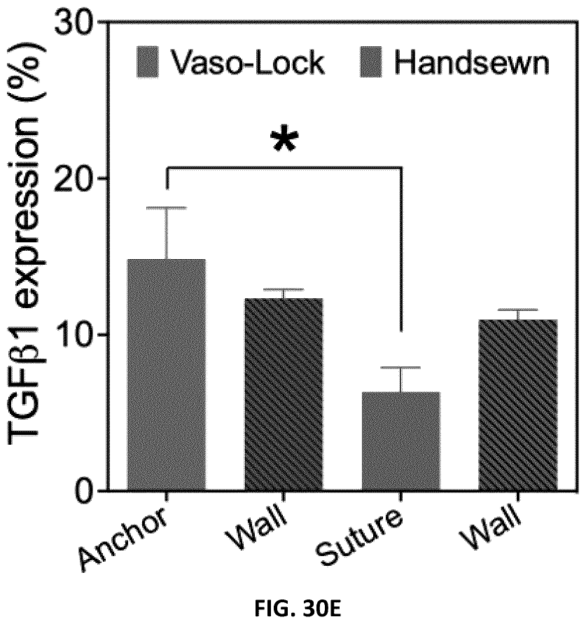

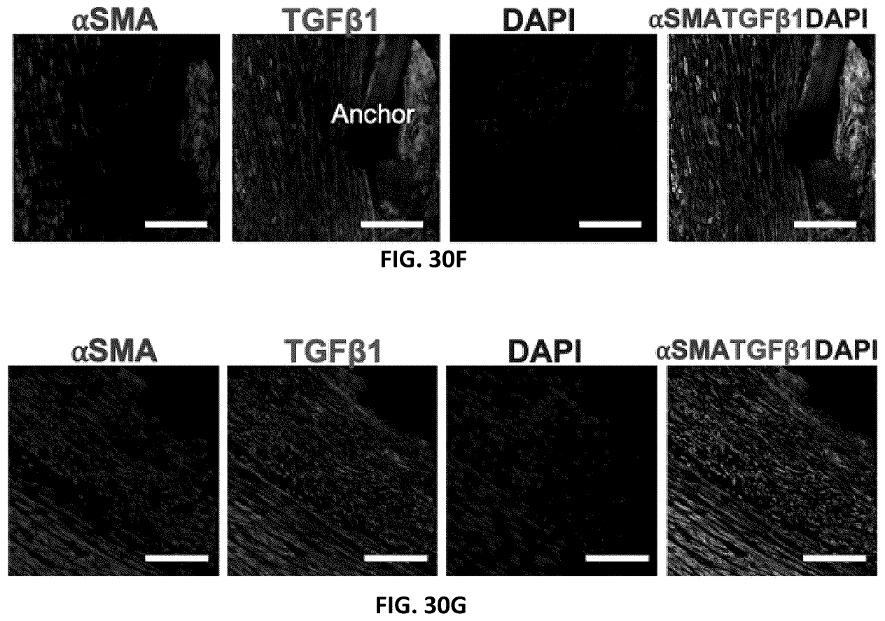

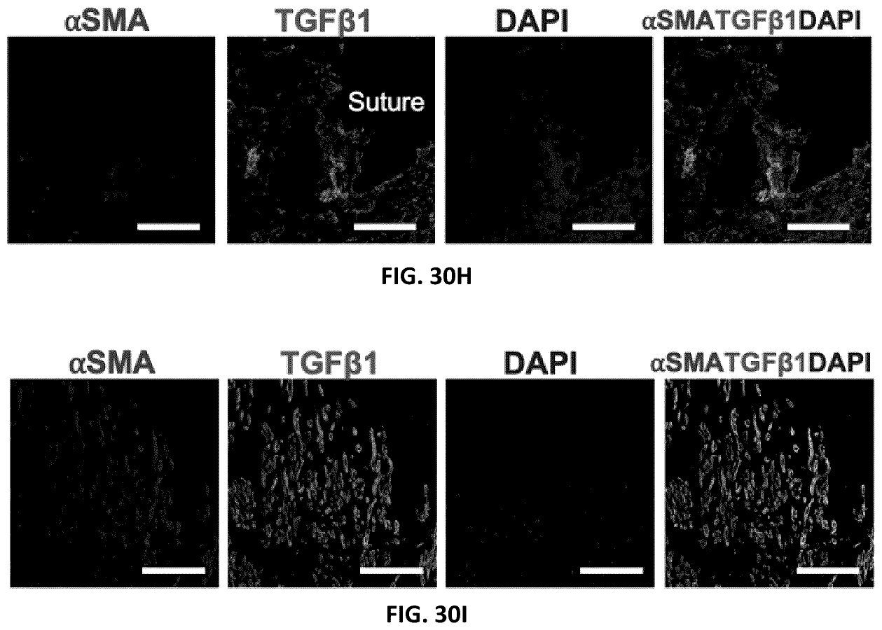

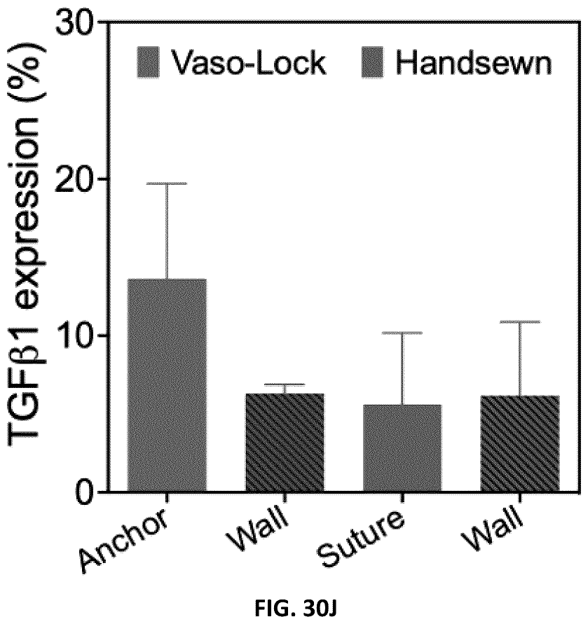

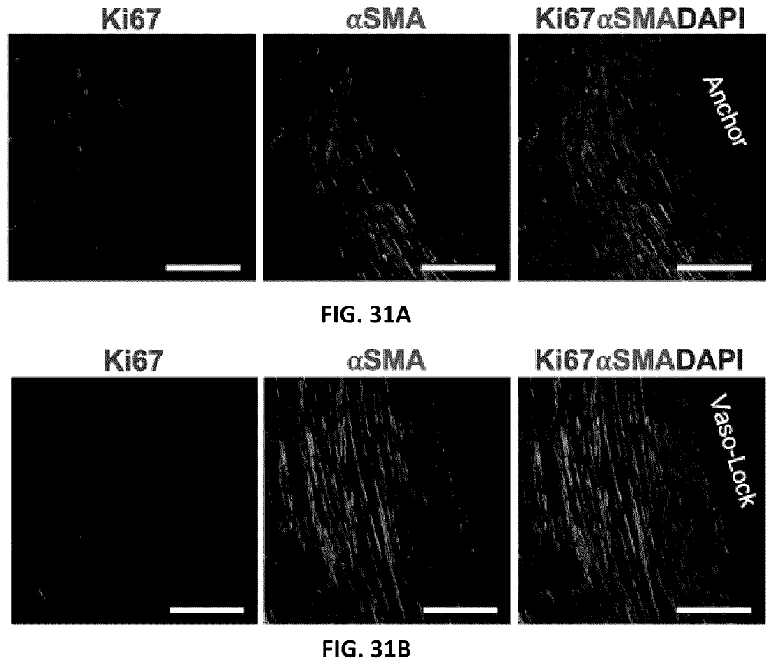

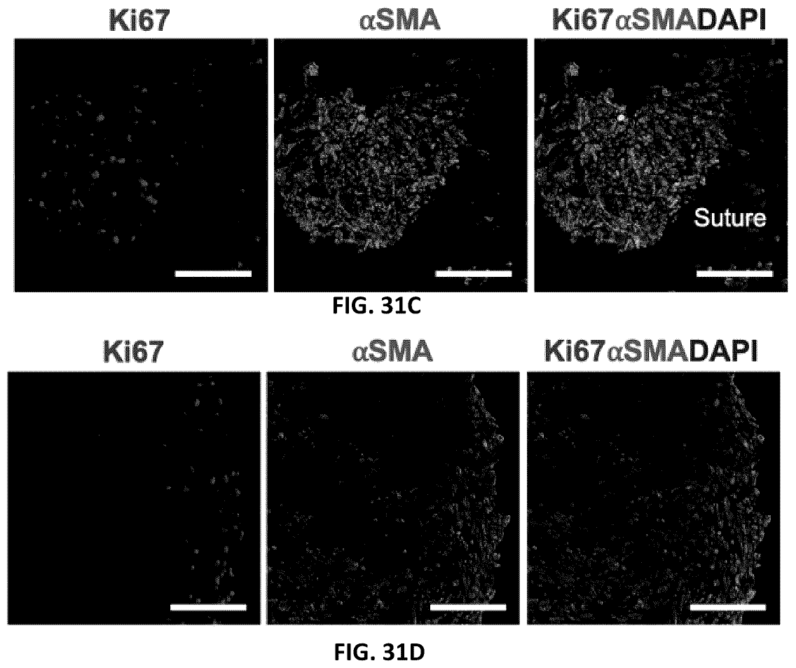

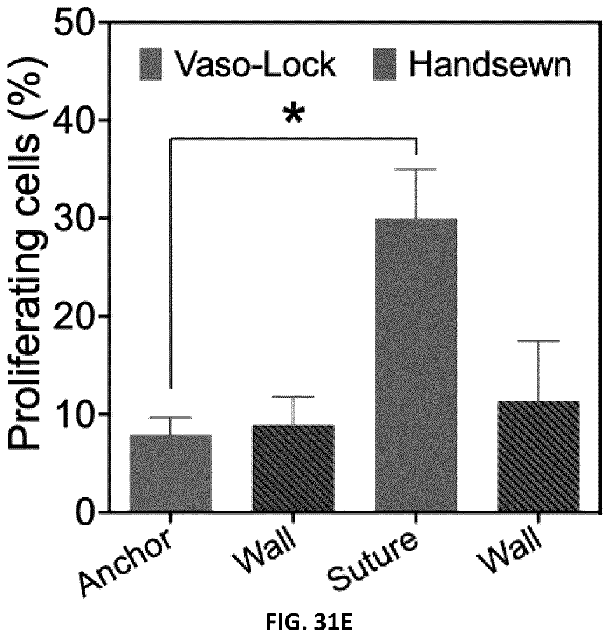

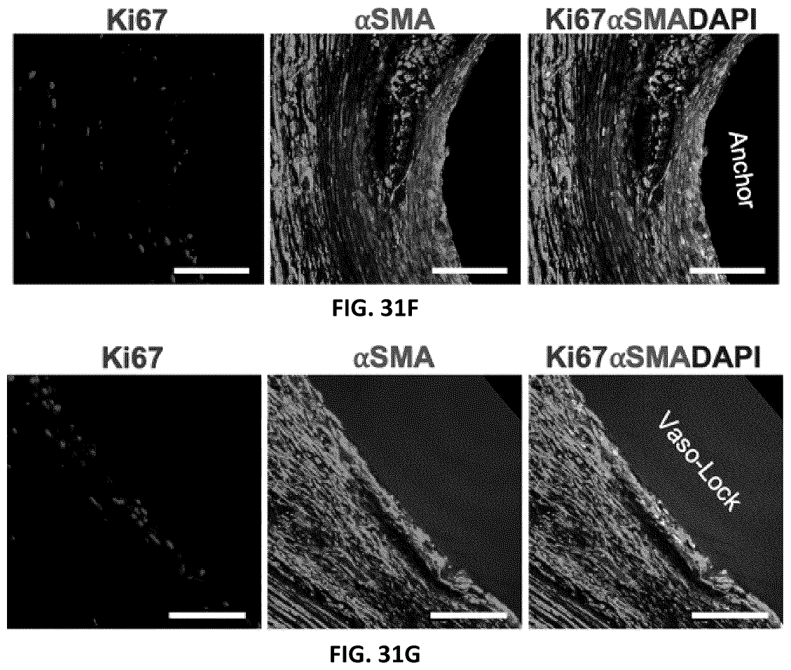

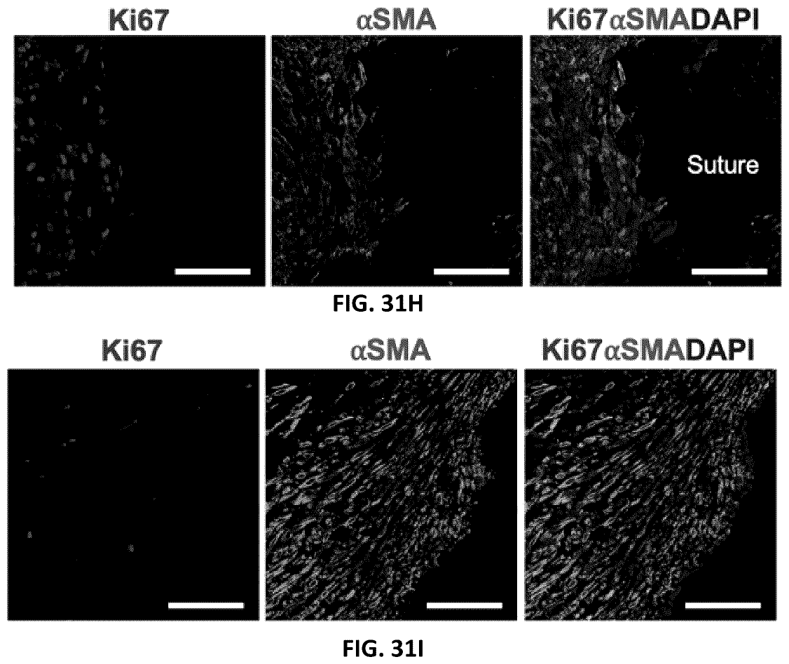

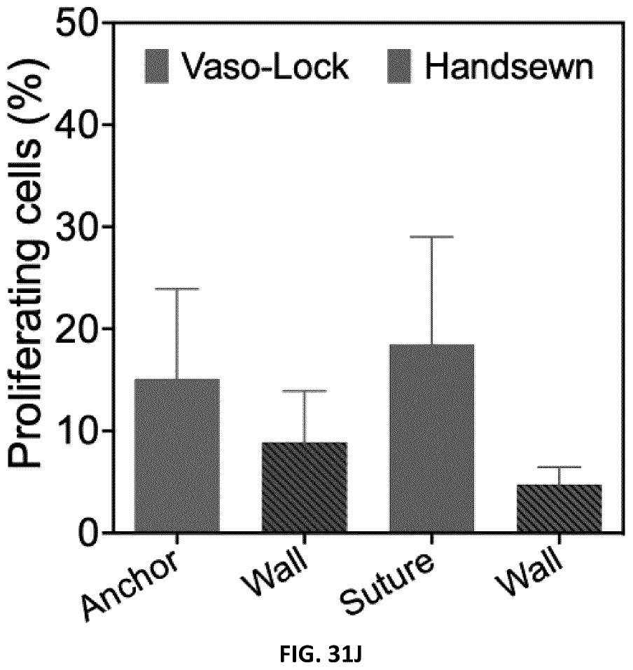

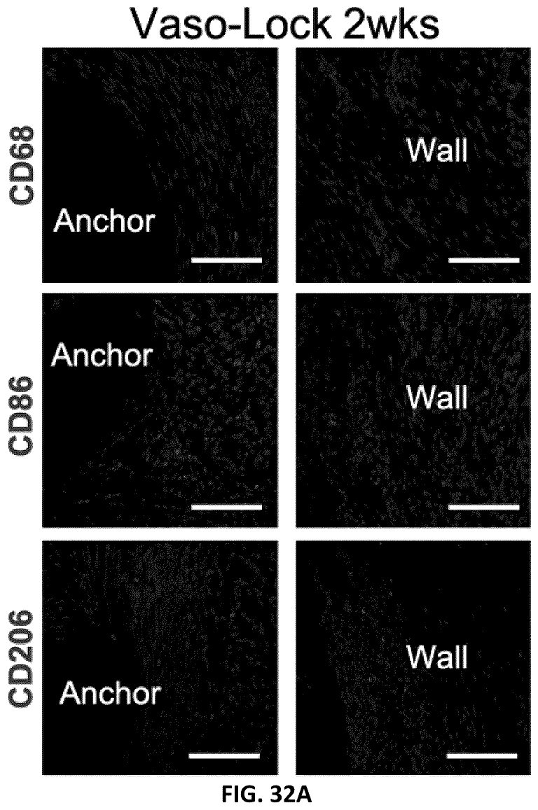

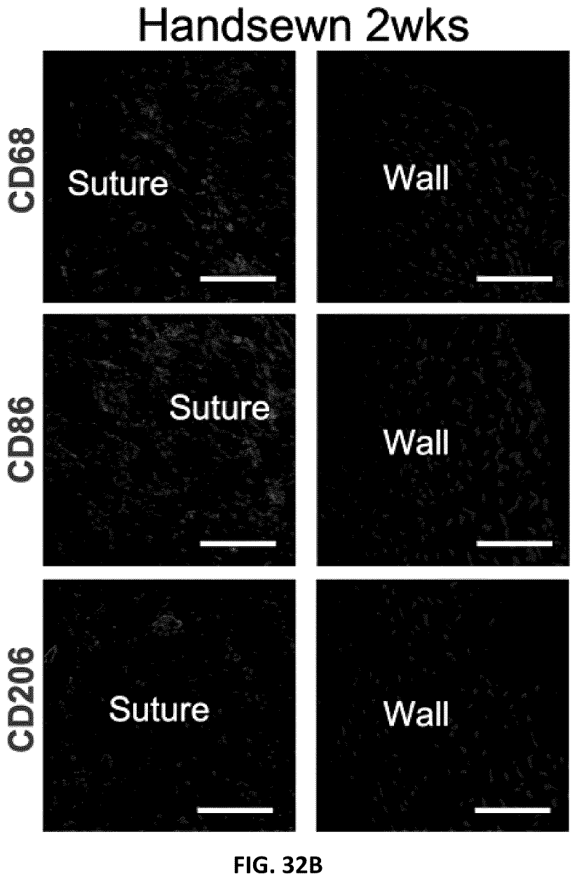

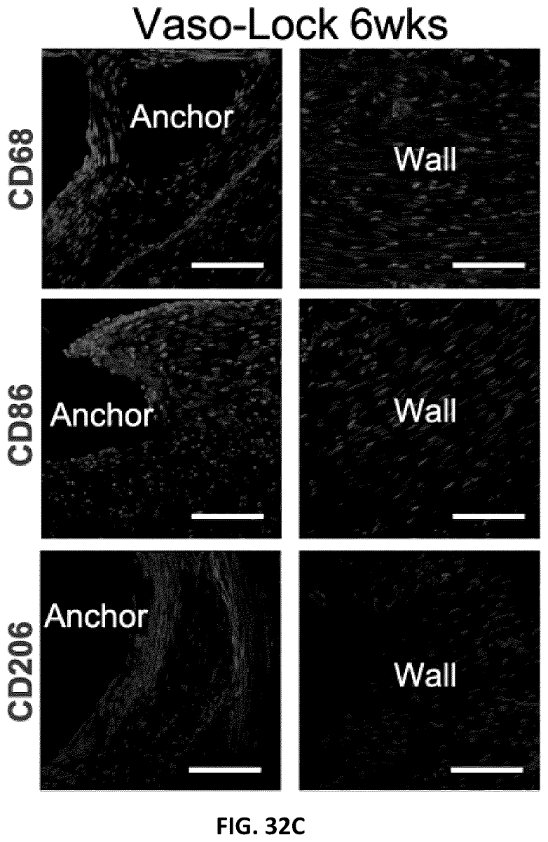

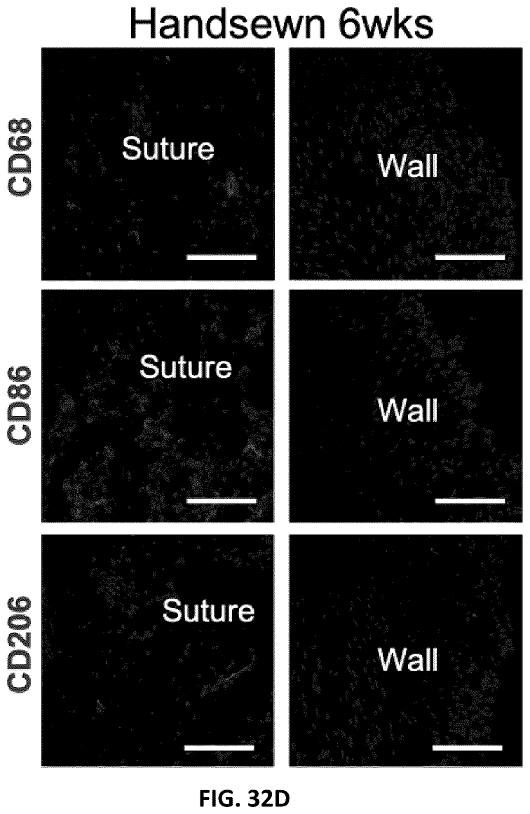

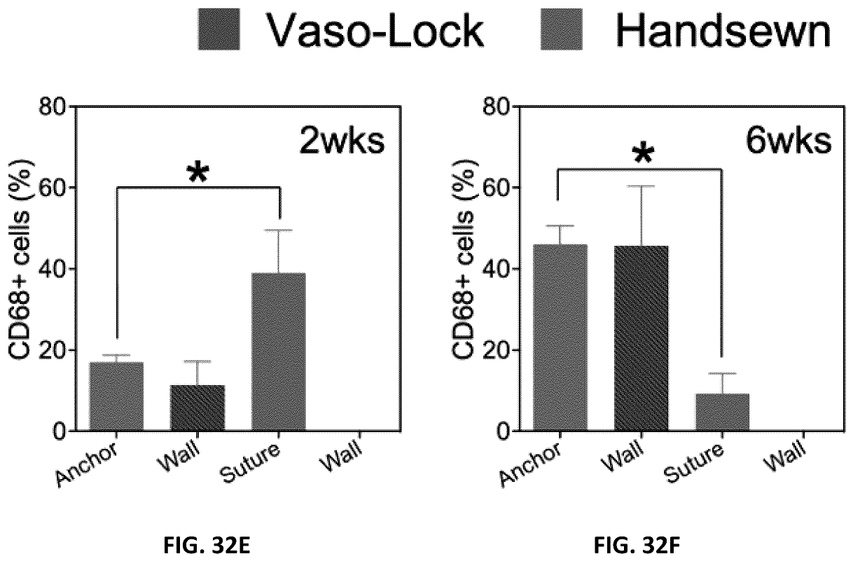

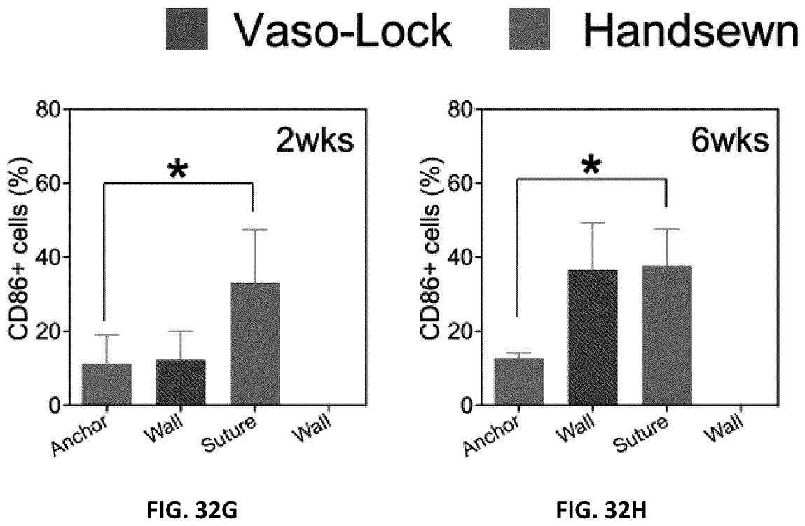

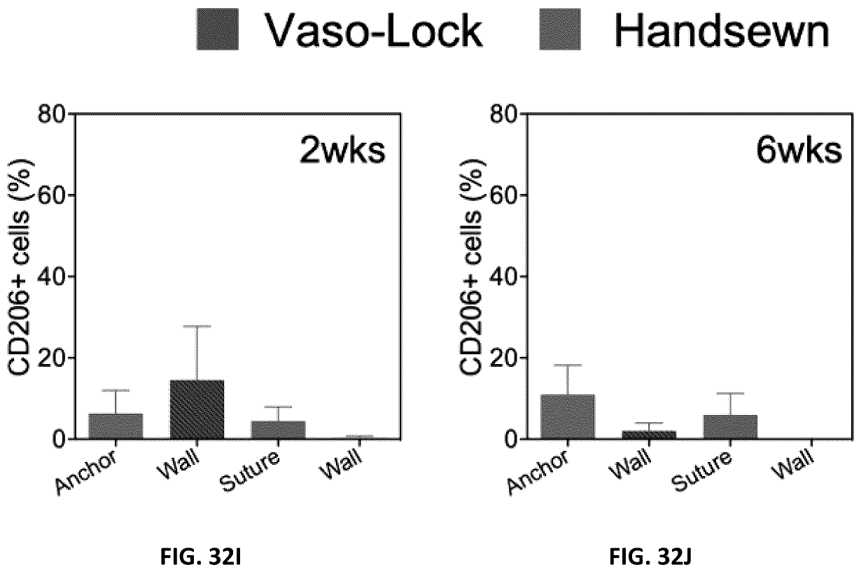

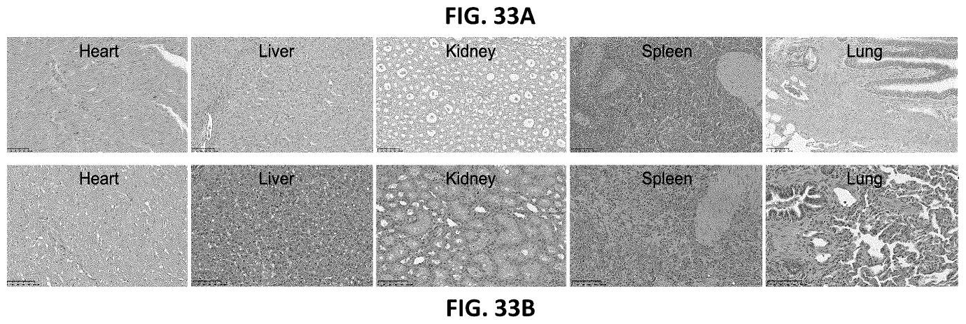

TGFβ1 expression analysis was then performed at the anastomosis sites in Weeks 2 and 6. Vaso-Lock for anastomosis (FIGS. 30A and B) and hand-sewn anastomosis (FIGS. 30 C and D) sites were imaged at Week 2, and TGFβ1 expression (green) was quantified (FIG. 30E). Similarly, Vaso-Lock for anastomosis (FIGS. 30F and G) and hand-sewn anastomosis (FIGS. 30H and I) sites were imaged at Week 6, and TGFβ1 expression was quantified (FIG. 30J). Proliferating cells at the anastomosis sites were then evaluated in Weeks 2 and 6. Vaso-Lock for anastomosis (FIGS. 31A and B) and hand-sewn anastomosis (FIGS. 31C and D) sites were stained with Ki67 at Week 2, and the percentage of proliferating cells was quantified (FIG. 31E). In turn, Vaso-Lock for anastomosis (FIGS. 31F and G) and hand-sewn anastomosis (FIGS. 31H and I) sites were stained with Ki67 and imaged at Week 6, and the percentage of proliferating cells was quantified (FIG. 31J). The presence of macrophages at the anastomosis sites was then characterized in Weeks 2 and 6. Macrophages were identified by CD68 (Pan macrophages), CD86 (Inflammatory macrophages), and CD206 (pro-regenerative macrophages) at Weeks 2 and 6 (FIG. 32A-D). Finally, a systemic evaluation of various organs of pigs with Vaso-Lock for anastomosis (FIG. 33A) and hand-sewn anastomosis (FIG. 33B) was performed during Week 6.

What is claimed is:

1. An anastomosis device for joining a first and second end of a vascular vessel, comprising a polymer cylinder ending in opposed first and second vascular-retaining ends configured for insertion into the first and second ends of the vascular vessel respectively, each vascular-retaining end comprising a surface and a plurality of bristles protruding outward from the surface, the device further comprising at least one moiety covalently coupled to the surface, the at least one moiety selected from at least one anti-coagulant, at least one cell-specific binding peptide, and any combination thereof.

2. The device of claim 1, wherein the polymer comprises one of polyethylene terephthalate (PET), expanded polytetrafluoroethylene (ePTFE), and polyether ether-ketone (PEEK).

3. The device of claim 1, wherein the at least one anti-coagulant comprises heparin, a polysaccharide selected from cellulose, methylcellulose, hydroxymethyl cellulose, hydroxypropyl cellulose, hydroxypropylmethylcellulose, carboxymethylcellulose, sodium carboxymethylcellulose, cellulose sulfate, chitosan, chitosan salts, hyaluronic acid, hyaluronic acid salts, dextran sulfate, chondroitin sulfate, heparin, starch, any derivative thereof, and any combination thereof.

4. The device of claim 1, wherein the at least one cell-specific binding peptide comprises a sequence selected from any one of REDV (SEQ ID NO:1), HGGVRLY (SEQ ID NO:2), RGD, IKVAV (SEQ ID NO:3), PDSGR (SEQ ID NO:4), and YIGSR(SEQ ID NO:5).

5. The device of claim 1, wherein the at least one cell-specific binding peptide comprises a loop structure and further comprises a peptide sequence selected from any one of SEQ ID NOS: 6-28.

6. The device of claim 1, wherein the at least one cell-specific binding peptide comprises a linear structure and further comprises a peptide sequence selected from any one of SEQ ID NOS: 29-51.

7. The device of claim 1, wherein the surface further comprises surface modifications comprising maleimide.

8. The device of claim 7, wherein the at least one moiety is covalently coupled to the maleimide.


SEQUENCE LISTING (51 sequences)

SEQ ID NO: 1 - protein, 4 aa, synthetic construct
REDV

SEQ ID NO: 2 - protein, 7 aa, synthetic construct
HGGVRLY

SEQ ID NO: 3 - protein, 5 aa, synthetic construct
IKVAV

SEQ ID NO: 4 - protein, 5 aa, synthetic construct
PDSGR

SEQ ID NO: 5 - protein, 5 aa, synthetic construct
YIGSR

SEQ ID NO: 6 - protein, 9 aa, synthetic construct
CCRRGDWLC

SEQ ID NO: 7 - protein, 13 aa, synthetic construct
CCRRKLDAPTWLC

SEQ ID NO: 8 - protein, 12 aa, synthetic construct
CCRRPHSRNWLC

SEQ ID NO: 9 - protein, 7 aa, synthetic construct
CCREDVC

SEQ ID NO: 10 - protein, 10 aa, synthetic construct
CCHGGVRLYC

SEQ ID NO: 11 - protein, 12 aa, synthetic construct
CCRRIKVAVWLC

SEQ ID NO: 12 - protein, 12 aa, synthetic construct
CCRRIKVAVWLC

SEQ ID NO: 13 - protein, 12 aa, synthetic construct
CCRRPDSGRWLC

SEQ ID NO: 14 - protein, 12 aa, synthetic construct
CCRRPDSGRWLC

SEQ ID NO: 15 - protein, 13 aa, synthetic construct
CCRRYVVLPRWLC

SEQ ID NO: 16 - protein, 12 aa, synthetic construct
CCRRLDVPSWLC

SEQ ID NO: 17 - protein, 12 aa, synthetic construct
CCRRIDAPSWLC

SEQ ID NO: 18 - protein, 15 aa, synthetic construct
CCRREILDVPSTWLC

SEQ ID NO: 19 - protein, 14 aa, synthetic construct
CCRREDGIHELWLC

SEQ ID NO: 20 - protein, 19 aa, synthetic construct
CCRRKAFDITYVRLKFWLC

SEQ ID NO: 21 - protein, 16 aa, synthetic construct
CCRRFRHRNRKGYWLC

SEQ ID NO: 22 - protein, 14 aa, synthetic construct
CCRRKRLDGSVWLC

SEQ ID NO: 23 - protein, 16 aa, synthetic construct
CCRRFRHRNRKGYWLC

SEQ ID NO: 24 - protein, 20 aa, synthetic construct
CCRRGQVFHVAYVLIKFWLC

SEQ ID NO: 25 - protein, 20 aa, synthetic construct
CCRRGTNNWWQSPSIQNWLC

SEQ ID NO: 26 - protein, 20 aa, synthetic construct
CCRRLWVTVRSQQRGLFWLC

SEQ ID NO: 27 - protein, 20 aa, synthetic construct
CCRRWVTVTLDLRQVFQWLC

SEQ ID NO: 28 - protein, 19 aa, synthetic construct
CCRRVLIKGGRARKHVWLC

SEQ ID NO: 29 - protein, 4 aa, synthetic construct
CRGD

SEQ ID NO: 30 - protein, 7 aa, synthetic construct
CKLDAPT

SEQ ID NO: 31 - protein, 6 aa, synthetic construct
CPHSRN

SEQ ID NO: 32 - protein, 5 aa, synthetic construct
CREDV

SEQ ID NO: 33 - protein, 8 aa, synthetic construct
CHGGVRLY

SEQ ID NO: 34 - protein, 6 aa, synthetic construct
CIKVAV

SEQ ID NO: 35 - protein, 6 aa, synthetic construct
CYIGSR

SEQ ID NO: 36 - protein, 6 aa, synthetic construct
CPDSGR

SEQ ID NO: 37 - protein, 8 aa, synthetic construct
CLNSSQPS

SEQ ID NO: 38 - protein, 8 aa, synthetic construct
CRYVVLPR

SEQ ID NO: 39 - protein, 6 aa, synthetic construct
CLDVPS

SEQ ID NO: 40 - protein, 6 aa, synthetic construct
CIDAPS

SEQ ID NO: 41 - protein, 9 aa, synthetic construct
CEILDVPST

SEQ ID NO: 42 - protein, 8 aa, synthetic construct
CEDGIHEL

SEQ ID NO: 43 - protein, 13 aa, synthetic construct
CKAFDITYVRLKF

SEQ ID NO: 44 - protein, 10 aa, synthetic construct
CFRHRNRKGY

SEQ ID NO: 45 - protein, 8 aa, synthetic construct
CKRLDGSV

SEQ ID NO: 46 - protein, 10 aa, synthetic construct
CFRHRNRKGY

SEQ ID NO: 47 - protein, 14 aa, synthetic construct
CGQVFHVAYVLIKF

SEQ ID NO: 48 - protein, 14 aa, synthetic construct
CGTNNWWQSPSIQN

SEQ ID NO: 49 - protein, 14 aa, synthetic construct
CLWVTVRSQQRGLF

SEQ ID NO: 50 - protein, 14 aa, synthetic construct
CWVTVTLDLRQVFQ

SEQ ID NO: 51 - protein, 13 aa, synthetic construct
CVLIKGGRARKHV